(19) United States
(12) Patent Application Publication
(10) Pub. No.: US 2008/0318905 A1
(43) Pub. Date: Dec. 25, 2008

(54) Prodrugs and methods of making and using the same

(76) Inventors: Naweed Muhammad, Fremont, CA (US); Keith R. Bley, Mountain View, CA (US)

Correspondence Address: MORRISON & FOERSTER LLP, 755 PAGE MILL RD, PALO ALTO, CA, 94304-1018, US

(21) Appl. No.: 11/999,660

(22) Filed: Dec. 5, 2007

Related U.S. Application Data
(60) Provisional application No. 60/873,519, filed on Dec. 5, 2006.

(51) Int. Cl. A61K 31/675; C07F 9/6527; C07F 9/59; C07F 9/553; C07F 9/6533; C07F 9/09; C07F 9/645

(52) U.S. Cl. 514/81; 514/89; 540/476; 558/166; 540/487; 544/88; 546/23; 546/22

(57) ABSTRACT

Prodrugs of parent drugs and methods of making and using the same are described. The prodrugs comprise an amine-containing parent drug moiety and a prodrug moiety, such as methoxyphosphonic acid or ethoxyphosphonic acid. The prodrugs may be employed in therapy for the treatment of various indications, such as pain, and in methods of decreasing the abuse potential of abuse-prone drugs and/or delaying the onset of parent drug activity and/or prolonging parent drug activity as compared to administration of a parent drug.

### CROSS-REFERENCE TO RELATED APPLICATIONS

This application claims priority to U.S. Provisional Application No. 60/873,519 filed Dec. 5, 2006.

### STATEMENT REGARDING FEDERALLY SPONSORED RESEARCH OR DEVELOPMENT

Not applicable.

### BACKGROUND OF THE INVENTION

Many drugs that are therapeutically beneficial have one or more undesirable characteristics that limit the use of the drug in therapy. For example, administration of certain drugs is accompanied by undesirable side effects. Some drugs have a short half-life and others are unstable or have a limited shelf life. Still other drugs, while therapeutically effective, have the potential for abuse.

Abuse-prone drugs constitute a considerable spectrum of drugs with applications in diverse therapeutic areas such as pain, insomnia, narcolepsy, depression, attention-deficit disorder, attention-deficit hyperactivity disorder, panic anxiety disorder, anesthesia, and weight loss. Classical classification into opioids, stimulants, benzodiazepines, and anorexiants covers most of the addictive drugs with abuse potential. While therapeutic significance of these drugs is immense, their use is associated with high degree of concern and reluctance on part of prescribing physician, dispensing pharmacist and those closely associated with the patient and often the patient himself/herself.

Opioid analgesics, one of the widely used abuse-prone drugs, are a mainstay of pain management. For example, they may be used to manage pain due to traumatic injury or surgery, pain produced by chronic inflammatory conditions such as osteoarthritis, rheumatoid arthritis and lower back pain. They may also be used to treat pain due to mixed nociceptive/neuropathic etiologies, such as cancer or fibromyalgia. Opioids may also be used to manage neuropathic pain, including pain associated with diabetic neuropathy, postherpetic neuralgia, HIV/AIDS, traumatic injury to nerves, complex regional pain syndrome, trigeminal neuralgia, erythromelalgia and phantom pain.

Despite their clinical advantages, the potential for opioid abuse is a major problem, both in substance and perception. Consequently, opioid abuse not only adversely affects abusers' health, safety and positive role in society, but also skews prescribing and dispensing practices of physicians and pharmacists, and can lead to a myriad of societal undesirable consequences. According to a recent study, an estimated 31.8 million Americans have used pain relievers non-medically in their lifetimes, up from 29.6 million in 2002. For instance, persons reporting lifetime non-medical use of a controlled-release form of oxycodone hydrochloride, OxyContin®, increased in the United States from 1.9 to 3.1 million persons between 2002 and 2004 (see, Nonmedical Users of Pain Relievers: Characteristics of Recent Initiatives, by the National Survey on Drug Use and Health, 2006).

Non-medical use of opioids remains a global problem, which may lead to the potential for undertreatment of pain. In a recent survey, abuse and addiction were the highest concerns (84% and 79%, respectively) expressed by physicians regarding the prescription of opioid analgesics, as compared to adverse effects, tolerance or medication interactions (68%, 61%, and 32%, respectively) (Survey of select practices by primary physicians on the opioids chronic pain, Curr. Med. Res. Opin. 2006. 22(9):1859-1865). Some primary care physicians reportedly hesitate to prescribe Schedule II opioids for 24-hour use in chronic nonmalignant pain, a condition that requires sustained analgesia day and night (See, Opioids for chronic nonmalignant pain. Attitudes and practices of primary care physicians in the UCSF/Stanford Collaborative Research Network. University of California, San Francisco J Fam. Pract. 2001. 50(2):145-151). According to one study of Canadian dispensing practice, 23% of primary care physicians and 35% of general practitioners reportedly would never prescribe opioids even for severe pain (see, Attitudes toward opioid use for chronic pain: a Canadian physician survey Pain Res. Manag. 2003. 8(4):189-194). Additional literature also points to the reluctance of physicians to prescribe opioids (Oncologists and primary care physicians' attitudes toward pain control and morphine prescribing in France, Cancer 1995. 76(11):2375-2382 and Morphine prescription to terminal cancer patients suffering from severe pain: results of a French survey, Bull Cancer. 2005. 92(7):733-740).

Even if an individual does not experience problems with opioid abuse, there are other drawbacks that can accompany existing opioid therapies, such as problems associated with inconvenient dosing schedules, risk of producing rapid overdoses, inability to deliver adequate dose levels in small dose volumes and not being suitable for prolonged or sustained delivery of opioid analgesics. Existing opioid therapies that require high local concentrations of the drug in the gastrointestinal tract and long-term use of opioid analgesics (e.g., in cancer and other chronic pain patients) often lead to adverse effects such as nausea, vomiting and constipation. The requirement for high dose for certain opioids also increases the probability that a rapid bolus exposure will occur and cause respiratory distress, dizziness, tiredness, somnolence, nausea and/or vomiting (See, e.g., “Are peripheral opioid antagonists the solution to opioid side effects?” Anesth. Analg. 2004. 98: 116-122.).

Various formulations and devices have been developed in an attempt to alleviate the abuse potential and/or other adverse side effects that can accompany opioid use. For instance, abuse-resistant tablets containing opioid receptor agonists and antagonists have been developed, wherein the antagonist is bioavailable only upon crushing or tampering with the tablet, as occurs when a drug abuser is seeking to extract the opiate from a sustained release formulation containing large amounts of drug. Opioid receptor antagonists have also been used to block the action of opioid agonists, such as when an overdose occurs. Although opioid antagonists can be used clinically to reverse the effects of opioid agonist overdoses, reduce the adverse effects associated with high concentrations of opioid agonists in the gastrointestinal tract, or to combat opioid or other recreational drug addictions, avoiding these adverse effects and the subsequent need for opioid receptor antagonists would be highly preferred. Other formulations have been reported to address opioid abuse potential, such as the opioid/fatty acid or fatty amine formulations described in U.S. Patent Publication No. 2005/0281748, or the inclusion of emetic agents in sustained-release oral formulations (e.g., ACUROX™ Tablets (oxycodone HCl and niacin) in development by Acura Pharmaceuticals.

Another alternative to conventional opioids has been the development of opioid prodrugs or analogs. Prodrugs and/or analogs of parent drug compounds may exhibit different pharmacological properties than the parent drug and may reduce the number or severity of problems associated with the parent drug compound, such as solubility, site specificity, stability, toxicity and sustained activity. For instance, U.S. Patent Publication No. 2004/0204434 describes prodrugs for use in lowering the abuse potential and extending the duration of action of a drug, such as oxycodone. U.S. Pat. Nos. 6,225,321 and 6,703,398 describe nalbuphine prodrugs and polyester derivatives.

Benzodiazepines such as diazepam, tetrazepam, lorazepam, nitrazepam and many other drugs with similar indications such as zolpidem, zaleplon, zopiclone have an important role in alleviating psychological or sleep disorders. While the low toxicity of benzodiazepines makes them a good choice for such disorders, their abuse potential makes physicians hesitant to prescribe them. This class of drugs is abused either to produce euphoria or altered state of consciousness, or to subside withdrawal symptoms of other addictive drugs. However, use of benzodiazepines to produce euphoria has been reported to be a more common issue (see Woody G. E., et al “Diazepam use by patients in methadone program: how serious a problem? J. Psychedelic Drugs 1975, 7: 373-379). Abuse potential of a drug when measured in terms of Relative High, Street Value and Use Again Value, diazepam was found to have highest abuse potential among other benzodiazepines (see O'Brien C. P. “Benzodiazepine Use, Abuse and Dependence J. Clin. Psychiatry 2005, 66(suppl. 2): 28-33).

Central nervous system (CNS) stimulants, such as amphetamine, methamphetamine and methylphenidate, are indicated for attention-deficit hyperactivity disorder (ADHD) and can be used to manage depression or as adjunctive pain therapies. ADHD is characterized by impulsivity, inattention and a persistent pattern of abnormally high levels of activity not observed in normal individuals with comparable levels of development. Methylphenidate (Ritalin™) can be a valuable medicine, for adults as well as children with ADHD. Methylphenidate and psychotherapy can improve the abnormal behaviors of ADHD, as well as the self-esteem, cognition, and social and family function of the patient. (see Konrad, K. et al “Differential Effects of Methylphenidate on Attentional Functions in Children With Attention-Deficit/Hyperactivity Disorder”. J. Am. Acad. Child Adolesc. Psychiatry, 2004, 43: 191-198). According to Monitoring the Future (MTF) survey, funded by the National Institute on Drug Abuse, National Institutes of Health, DHHS, and conducted annually by the University of Michigan's Institute for Social Research, in year 2006 annual illicit use of methylphenidate and methamphetamine in 8^{th}-graders, 10^{th}-graders, and 12^{th}-graders was 2.7, 3.2, and 4.4%, respectively. Amphetamine use, sadly, is relatively much higher with 7.3, 11.2, and 12.4% in 8^{th}-graders, 10^{th}-graders, and 12^{th}-graders, respectively. (see http://www.monitoringthefuture.org/data/06data/pr06t1.pdf, accessed Dec. 2, 2007). Signs and symptoms of acute methylphenidate overdosage, resulting principally from overstimulation of the CNS and from excessive sympathomimetic effects, may include the following: vomiting, agitation, tremors, hyperreflexia, muscle twitching, convulsions (may be followed by coma), euphoria, confusion, hallucinations, delirium, sweating, flushing, headache, hyperpyrexia, tachycardia, palpitations, cardiac arrhythmias, hypertension, mydriasis, and dryness of mucous membranes. While overdose death is not common, it has happened.

Some prescription anorexiants—for example, phenmetrazine—have high abuse potential and are accordingly classified by the DEA as Schedule II. A derivative of phenmetrazine, phendimetrazine has a lower abuse potential, and is thus classified by the DEA as Schedule III.

Abuse deterrence also has been attempted by incorporating into the formulation, an irritant which is released only when original dosage is tempered or used in a non-prescribed manner (e.g., via an alternate route of administration such as snorting, chewing or intravenous injection). E.U. Patent application No. 1392270 (WO02094254) describes pharmaceutical composition intended for oral use, which contains besides effective ingredient(s) and other typical fillers and excipients, capsaicin—a highly irritating substance—which causes irritation of mucous membranes if the tablets used for abusive snorting, injection, or ingestion.

Altering pharmacokinetic properties, particularly a delay in onset of action, of opioids, benzodiazepines, stimulants or anorexiants, and other abuse-prone drugs containing a tertiary or secondary amine functional group may render these drugs less prone to abuse while retaining their therapeutic utility.

As reported in U.S. Pat. No. 5,985,856 (see, e.g., columns 11-13 of U.S. Pat. No. 5,985,856) and in Krise, J., et al, “Novel prodrug approach for tertiary amines: synthesis and preliminary evaluation of N-phosphonooxymethyl prodrugs,” J. Med. Chem. 1999 42(16): 3094-3100, parent compounds bearing tertiary amines may be modified at the amine position with methoxyphosphonic acid to impart improved water solubility characteristics. An in vivo study reported therein discloses the ability of a cinnarizine prodrug to be converted to the parent drug cinnarizine in a beagle dog following i.v. administration. Compounds bearing secondary amines have also been subjected to derivatization with a phosphate group. For example, the anticonvulsant phenyloin (Dilantin®) has been converted to fosphenyloin (Cerebyx®) by converting a secondary amine at position 3 of imidazolidine-2,4-dione ring to a tertiary amine by phosphonooxymethyl group. This change led to improved profile of fosphenyloin with low incidences of phlebitis (see, Venous irritation related to intravenous administration of phenyloin versus fosphenyloin Pharmacotherapy 1994; 14:47-52).

Despite the advances in drug therapy aimed at reducing or eliminating one or more of the undesirable characteristics of certain parent drugs, there remains a significant interest in, and need for, additional or alternative approaches and therapies which preferably address one or more of the problems associated with existing therapies.

### BRIEF SUMMARY OF THE INVENTION

Prodrugs that impart a favorable characteristic to a parent drug offer potential new therapies for a variety of indications and symptom management. Prodrugs can offer greater stability and/or more favorable formulation characteristics than a parent drug, which can be useful in increasing shelf life or lessoning the severity of conditions under which a formulated drug must be stored. In some instances, a prodrug may be less susceptible to in vivo degradation and exhibit a greater half-life than its parent drug. A prodrug with a greater half-life is likely to require less frequent dosing and/or reduced dose than that of a parent drug, which can be particularly important when administration of a parent drug is accompanied by unfavorable side effects, such as nausea or dosing frequency promotes non-compliance. Still further, a prodrug with different physicochemical characteristics than a parent drug may be more amenable to certain drug delivery routes.

The present invention relates to prodrugs and methods of their use in therapy. The prodrugs employ a parent drug having an amine functionality and a prodrug moiety, preferably a moiety of the formula (IA), (IB) or (2), where the prodrug moiety is bound to the parent drug moiety via a covalent bond to the amine functional group on the parent drug. In one aspect of the invention, the prodrugs detailed herein exhibit one or more favorable characteristics over their parent drug. In one variation, the invention relates to an opioid prodrug that exhibits one or more favorable characteristics over its parent opioid. In another variation, the invention relates to a prodrug of a compound that affects the central nervous system (“CNS drugs”) where the prodrug exhibits one or more favorable characteristics over its parent CNS drug. In yet another variation, the invention relates to a stimulant prodrug that exhibits one or more favorable characteristics over its parent stimulant. In still another variation, the invention relates to a benzodiazepine prodrug that exhibits one or more favorable characteristics over its parent benzodiazepine. The invention also embraces an anorexiant prodrug that exhibits one or more favorable characteristics over its parent anorexiant. The favorable characteristic of a prodrug may be, but is not limited to, decreased abuse potential as compared to its parent drug. Other favorable characteristics of a prodrug may include, but are not limited to, decreased side effects, increased shelf life, increased half life, greater stability, more favorable formulation characteristics, and suitability for dosage form(s) for which the parent drug is not suitable such as sustained release, delayed release, and/or site-specific delivery.

Prodrugs of the invention, such as prodrugs of abuse-prone parent drugs (APDs), are described that may address one or more existing problems associated with the parent drugs, which may be but are not limited to opioids, benzodiazepines, stimulants or anorexiants. In one variation, the parent drug is an abuse-prone parent drug, or APD. Also described are methods of using prodrugs of the invention, including methods of treating pain or psychic disorders and, where the parent drug is an ADP, methods of decreasing the abuse potential of an APD. Methods of delaying the onset of a parent drug's activity and/or prolonging its activity when compared to administration of a parent drug are also embraced by the invention.

Prodrugs of the invention may include the prodrug moiety -alkyl-OP(O)(OH)₂, such as the prodrug moieties —CH₂CH₂OP(O)(OH)₂, —CH(CH₃)OP(O)(OH)₂ and —CH₂OP(O)(OH)₂ and in one variation the prodrug moiety is attached to a parent drug via a nitrogen, such as from an amine (e.g., a tertiary amine) present on a parent drug. In one variation, the prodrug is N-phosphonooxymethyl levorphanol or N-phosphonooxyethyl levorphanol. In one variation, the methods described herein employ the prodrug N-phosphonooxymethyl levorphanol or N-phosphonooxyethyl levorphanol.

The prodrug may be, and any of the methods described herein may use, a prodrug of the formula (I):

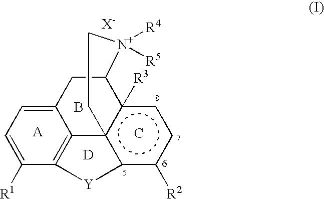

or of the formula (II):

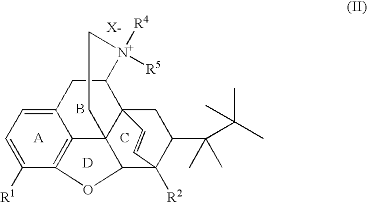

or of the formula (III):

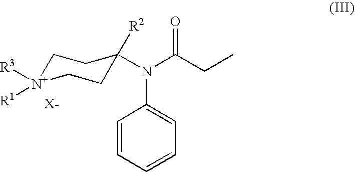

or of the formula (IV):

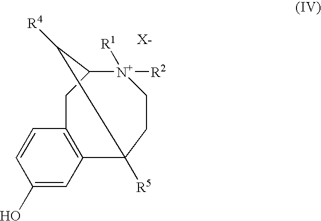

or of the formula (V):

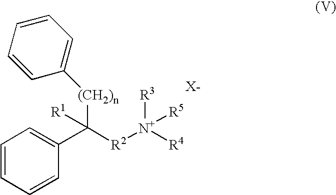

or of formula (VI)

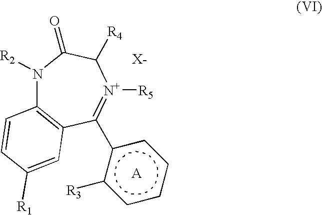

or of formula (VII)

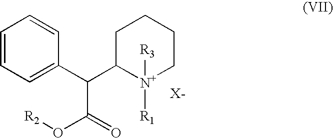

or of formula (VIII)

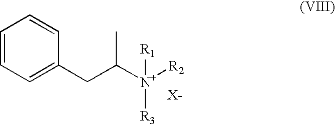

or of formula (IX)

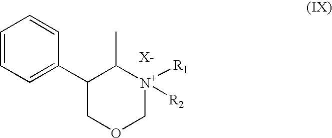

where the substituents for formulae (I)-(IX) are as described herein.

Novel prodrugs, including novel APD prodrugs, and methods of using the same are embraced by this invention. In one variation, the prodrug is a compound comprising a parent drug moiety and a prodrug moiety of the formula:

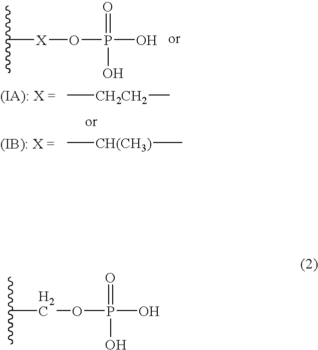

In one variation, when the prodrug moiety is of the formula (2), the parent drug moiety is not a moiety of a parent drug listed in columns 11-14 of U.S. Pat. No. 5,985,856, such as levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, when the prodrug moiety is of the formula (2), the parent drug moiety may be any suitable parent drug moiety, including a moiety of a parent drug listed in columns 11-14 of U.S. Pat. No. 5,985,856, such as levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation, when the prodrug moiety is of the formula (1A), the parent drug moiety is not a moiety of a parent drug listed in columns 11-14 of U.S. Pat. No. 5,985,856, such as levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation, when the prodrug moiety is of the formula (1A), the parent drug moiety may be any suitable parent drug moiety, including a moiety of a parent drug listed in columns 11-14 of U.S. Pat. No. 5,985,856, such as levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation, when the prodrug moiety is of the formula (1B), the parent drug moiety is not a moiety of a parent drug listed in columns 11-14 of U.S. Pat. No. 5,985,856, such as levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, when the prodrug moiety is of the formula (1B), the parent drug moiety may be any suitable parent drug moiety, including a moiety of a parent drug listed in columns 11-14 of U.S. Pat. No. 5,985,856, such as levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, the prodrug is a compound comprising a parent drug moiety and a prodrug moiety of the formula (1A). In another variation, the prodrug is a compound comprising a parent drug moiety and a prodrug moiety of the formula (1B). In still another variation, the prodrug is a compound comprising a parent drug moiety and a prodrug moiety of the formula (2).

In one variation, the prodrug moiety is (1A), (1B) or (2) and the parent drug is dihydrocodeine, bromazepam, clorazepate, flunitrazepam or oxazolam. In one variation, the prodrug moiety is (1A), (1B) or (2) and the parent drug is 4-(4-(4-chlorophenyl)-4-hydroxycyclohexyl)-N,N-diethyl-2,2-diphenylbutanamide or 4-(4-(4-chlorophenyl)-4-hydroxycyclohexyl)-N-ethyl-N-methyl-2,2-diphenylbutanamide.

The prodrug may be, and any of the methods described herein may use, an opioid prodrug of the formula (I):

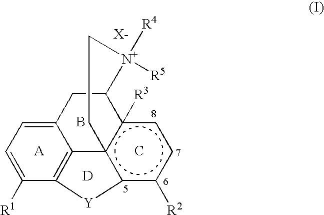

wherein R¹ is selected from the group consisting of hydrogen, C₁-C₁₀ alkanoate, hydroxyl and a substituted or unsubstituted C₁-C₁₀ alkyl and substituted or unsubstituted C₁-C₁₀ alkoxy; R² is selected from the group consisting of hydrogen, ═O, hydroxyl, a substituted or unsubstituted C₁-C₁₀ alkyl, a substituted or unsubstituted C₂-C₁₀ alkenyl and a substituted or unsubstituted C₁-C₁₀ alkoxy; R³ is selected from the group consisting of hydrogen, hydroxyl, C₁-C₁₀ alkanoate, a substituted or unsubstituted C₁-C₁₀ alkyl and a substituted or unsubstituted C₁-C₁₀ alkoxy; R⁴ is selected from a group consisting of hydrogen, C₁-C₁₀ alkanoate, a substituted or unsubstituted C₁-C₁₀ alkyl, a substituted or unsubstituted C₂-C₁₀ alkenyl and a substituted or unsubstituted C₁-C₁₀ alkoxy; R⁵ is the prodrug moiety (1A), (1B) or (2); Y is null or is selected from O and S; ring C has zero, one or two double bonds; X⁻ is a pharmaceutical acceptable anion; or any stereoisomer, salt, hydrate or solvate thereof. In one variation, ring C of formula (I) has zero double bonds. The opioid prodrug may be, and any of the methods described herein may use, a prodrug of the formula (I) where R¹ is hydroxyl, R² and R³ are hydrogen, R⁴ is methyl, R⁵ is the prodrug moiety (1A) or (1B) and Y is null or where R² is hydroxyl, R² and R³ are hydrogen, R⁴ is methyl, R⁵ is the prodrug moiety (2) and Y is null or where R¹ is methoxy, R² is ═O, R³ is hydroxyl, R⁴ is methyl, R⁵ is the prodrug moiety (1A) or (1B) and Y is oxygen or where R¹ is methoxy, R² is ═O, R³ is hydroxyl, R⁴ is methyl, R⁵ is the prodrug moiety (1A) or (1B) and Y is oxygen or where R¹ is hydroxyl, R² is ═O, R³ is hydroxyl, R⁴ is methyl, R⁵ is the prodrug moiety (1A) or (1B) and Y is oxygen or where R¹ is hydroxyl, R² is ═O, R³ is hydroxyl, R⁴ is methyl, R⁵ is the prodrug moiety (1A) or (1B) and Y is oxygen or where R¹ is methoxy, R² is hydroxyl, R³ is hydrogen, R⁴ is methyl, R⁵ is the prodrug moiety (1A) or (1B), Y is oxygen and C₇ and C₈ are connected by a double bond or where R¹ is hydroxyl, R² is hydroxyl, R³ is hydrogen, R⁴ is methyl, R⁵ is the prodrug moiety (1A) or (1B), Y is oxygen and C₇ and C₈ are connected by a double bond or where R¹ is hydroxyl, R² is ═O, R³ is hydroxyl, R⁴ is cyclopropylmethyl and Y is O or where R¹ is hydroxyl, R² is ═O, R³ is hydroxyl, R⁴ is propen-3-yl and Y is O or where R¹ is hydroxyl, R² is ethenyl, R³ is hydroxyl, R⁴ is cyclopropylmethyl and Y is O or where R¹ is hydroxyl, R² is hydroxyl, R³ is hydrogen, R⁴ is propen-3-yl and Y is O or where R¹ is hydroxyl, R² is hydroxyl, R³ is hydroxyl, R⁴ is cyclobutylmethyl and Y is O or where R¹ is hydroxyl, R² is hydrogen, R³ is hydrogen, R⁴ is cyclopropylmethyl and Y is null or where R¹ is hydroxyl, R² is hydrogen, R³ is hydroxyl, R⁴ is cyclopropylmethyl and Y is null or where R¹ is hydroxyl, R² is hydrogen, R³ is hydrogen, R⁴ is propen-3-yl and Y is null. In one variation, the opioid prodrug is of the formula (I) provided that when the prodrug moiety is of the formula (2), the opioid moiety is not a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation, the opioid prodrug is of the formula (I) and the prodrug moiety is of the formula (2) and the opioid moiety is a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, opioid prodrug is of the formula (I) and a prodrug moiety of the formula (1B) provided that opioid moiety is not a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, opioid prodrug is of the formula (I) and a prodrug moiety of the formula (1B) and the opioid moiety is a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, opioid prodrug is of the formula (I) and a prodrug moiety of the formula (1A) provided that opioid moiety is not a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, opioid prodrug is of the formula (I) and a prodrug moiety of the formula (1A) and the opioid moiety is a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In yet another variation, opioid prodrug is of the formula (I) and a prodrug moiety of the formula (1A) opioid moiety is a moiety of any known opioid or derivatives thereof. In yet another variation, opioid prodrug is of the formula (I) and a prodrug moiety of the formula (1A) opioid moiety is a moiety of any known opioid or derivatives thereof.

The prodrug may be, and any of the methods described herein may use, an opioid prodrug of the formula (II):

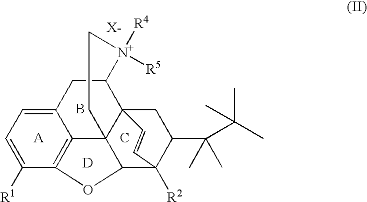

where R¹ is selected from the group consisting of hydrogen, hydroxyl, a substituted or unsubstituted C₁-C₁₀ alkyl and a substituted or unsubstituted C₁-C₁₀ alkoxy; R² is selected from the group consisting of hydrogen, hydroxyl, a substituted or unsubstituted C₁-C₁₀ alkyl, a substituted or unsubstituted C₂-C₁₀ alkenyl and a substituted or unsubstituted C₁-C₁₀ alkoxy; R⁴ is selected from a group consisting of hydrogen, a substituted or unsubstituted C₁-C₁₀ alkyl and a substituted or unsubstituted C₁-C₁₀ alkoxy; R⁵ is the prodrug moiety (1A), (1B) or (2); X⁻ is pharmaceutical acceptable anion; or any stereoisomer, salt, hydrate or solvate thereof. The opioid prodrug may be and any of the methods described herein may use a prodrug of the formula (II) where R¹ is hydroxyl, R² is methoxy, R⁴ is cyclopropylmethyl, R⁵ is methoxyphosphonic acid and or where R¹ is hydroxyl, R² is methoxy, R⁴ is cyclopropylmethyl, and R⁵ is ethoxyphosphonic acid. In one variation, R⁵ is a prodrug moiety of formula (1A), (1B) or (2).

The prodrug may be, and any of the methods described herein may use, an opioid prodrug of the formula (III):

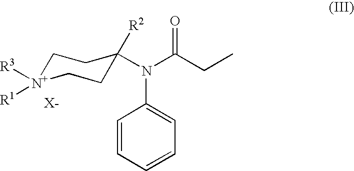

where R¹ is selected from the group consisting of hydroxyl, propylbenzene, ethylbenzene, 2-propylthiophene, methyl butyrate, 1-ethyl-4-ethyl-1H-tetrazol-5(4H)-one and 1-ethyl-4-propyl-1H-tetrazol-5(4H)-one, a substituted or unsubstituted C₁-C₁₀ alkyl and a substituted or unsubstituted C₁-C₁₀ alkoxy; R² is selected from the group consisting of hydrogen, ═O, hydroxyl, a substituted or unsubstituted C₁-C₁₀ alkyl and C₁-C₁₀ alkoxy, C₁-C₁₀ alkanoate, C₂-C₁₀ alkoxyalkyl; R³ is the prodrug moiety (1A), (1B) or (2); X⁻ is a pharmaceutically acceptable anion; or any stereoisomer, salt, hydrate or solvate thereof. The opioid prodrug may be and any of the methods described herein may use a prodrug of the formula (III) where R¹ is propylbenzene, R² is hydrogen, and R³ is methoxyphosphonic acid or where R¹ is propylbenzene, R² is hydrogen, and R³ is ethoxyphosphonic acid or where R¹ is 2-propylthiophene, R² is methoxy methyl, and R³ is methoxyphosphonic acid or where R¹ is 2-propylthiophene, R² is methoxy methyl, and R³ is ethoxyphosphonic acid or where R¹ is 1-ethyl-4-ethyl-1H-tetrazol-5(4H)-one, R² is methoxy methyl, and R³ is methoxyphosphonic acid or where R¹ is 1-ethyl-4-ethyl-1H-tetrazol-5(4H)-one, R² is methoxy methyl, and R³ is ethoxyphosphonic acid or where R¹ is methyl butyrate, R² is methyl formoate, and R³ is methoxyphosphonic acid or where R¹ is methyl butyrate, R² is methyl acetate, and R³ is ethoxyphosphonic acid. In a particular variation, R³ is a prodrug moiety of formula (1A), (1B) or (2).

The prodrug may be, and any of the methods described herein may use, an opioid prodrug of the formula (IV):

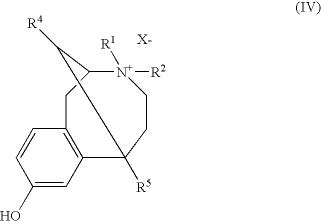

where R⁴ and R⁵ are independently alkyl; R² is the prodrug moiety (1A), (1B) or (2); R¹ is alkaryl or alkenyl and X⁻ is a pharmaceutically acceptable anion, and any stereoisomer, salt, hydrate or solvate thereof. The opioid prodrug may be and any of the methods described herein may use a prodrug of the formula (IV) where R⁴ and R⁵ are independently selected a substituted or unsubstituted C₁-C₅ alkyl; R² is the prodrug moiety (1A), (1B) or (2); R¹ is —(CH₂)ₙ-phenyl where n is selected from 1 to 5 or a C₂-C₁₀ alkenyl and X⁻ is a pharmaceutically acceptable anion, and any stereoisomer, salt, hydrate or solvate thereof.

The prodrug may be, and any of the methods described herein may use, an opioid prodrug of the formula (V):

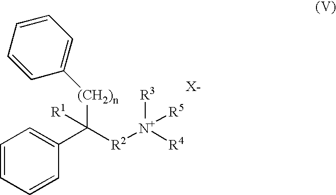

where R¹ is an alkanoate or a carbonylalkyl; R², R³ and R⁴ are independently a substituted or unsubstituted alkyl; R⁵ is the prodrug moiety (1A), (1B) or (2); n is an integer from 1 to 10 and X⁻ is a pharmaceutically acceptable anion, and any stereoisomer, salt, hydrate or solvate thereof. The opioid prodrug may be and any of the methods described herein may use a prodrug of the formula (V) where R¹ is propanoate or propionyl; R², R³ and R⁴ are independently a substituted or unsubstituted C₁-C₅ alkyl; R⁵ is the prodrug moiety (1A), (1B) or (2); n is an integer from 1 to 5 and X⁻ is a pharmaceutically acceptable anion, and any stereoisomer, salt, hydrate or solvate thereof.

The prodrug may be, and any of the methods described herein may use, a benzodiazepine prodrug of the formula (VI):

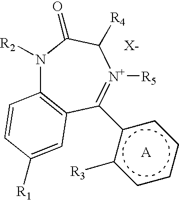

(VI)

where R¹ is a halogen, nitro group, —NR₂, —NHR, —NH₂, —SO₃H, —CF₃, —C(O)Cl, —C(O)OH, —C(O)R, —C(O)OR, —C(O)H or alkyl or hydrogen where R is a substituted or unsubstituted C₁-C₅ alkyl; R² is a hydrogen or a substituted or unsubstituted alkyl; R³ is hydrogen, halogen or a substituted or unsubstituted C₁-C₅ alkyl and R⁴ is a hydrogen, nitro group, hydroxyl, or oxygen; Ring A aromatic or in non-aromatic but has one or two double bonds; R⁵ is the prodrug moiety (1) or (2); X⁻ is a pharmaceutically acceptable anion, and any stereoisomer, salt, hydrate or solvate thereof. The benzodiazepine prodrug may be and any of the methods described herein may use a prodrug of the formula (VI) where R¹ is chloro or nitro group; R² is hydrogen or methyl, R³ is hydrogen or fluorine or chlorine and R⁴ is hydrogen, nitro group or oxygen; R⁵ is the prodrug moiety (1) or (2); X⁻ is a pharmaceutically acceptable anion, and any stereoisomer, salt, hydrate or solvate thereof.

In one variation, the benzodiazepine prodrug is of the formula (VI) provided that when the prodrug moiety is of the formula (2), the benzodiazepine moiety is not a moiety of a benzodiazepine selected from the group consisting of alprazolam, clorazepate, clonazepam, estazolam, flurazepam, halazepam, lorazepam, midazolam, oxazepam, quazepam, temazepam and triazolam. In another variation, the benzodiazepine prodrug is of the formula (VI) where the prodrug moiety is of the formula (2) and the benzodiazepine moiety is a moiety of a benzodiazepine selected from the group consisting of alprazolam, clorazepate, clonazepam, estazolam, flurazepam, halazepam, lorazepam, midazolam, oxazepam, quazepam, temazepam and triazolam. In another variation, benzodiazepine prodrug is of the formula (VI) and a prodrug moiety of the formula (1B) provided that benzodiazepine moiety is not a moiety of an benzodiazepine selected from the group consisting of alprazolam, clorazepate, clonazepam, estazolam, flurazepam, halazepam, lorazepam, midazolam, oxazepam, quazepam, temazepam and triazolam. In another variation, benzodiazepine prodrug is of the formula (VI), the prodrug moiety is of the formula (1B) and the benzodiazepine moiety is a moiety of a benzodiazepine selected from the group consisting of alprazolam, clorazepate, clonazepam, estazolam, flurazepam, halazepam, lorazepam, midazolam, oxazepam, quazepam, temazepam and triazolam. In another variation, benzodiazepine prodrug is of the formula (VI) and a prodrug moiety of the formula (1A) provided that benzodiazepine moiety is not a moiety of an benzodiazepine selected from the group consisting of alprazolam, clorazepate, clonazepam, estazolam, flurazepam, halazepam, lorazepam, midazolam, oxazepam, quazepam, temazepam and triazolam. In another variation, benzodiazepine prodrug is of the formula (VI), the prodrug moiety is of the formula (1A) and the benzodiazepine moiety is a moiety of a benzodiazepine selected from the group consisting of alprazolam, clorazepate, clonazepam, estazolam, flurazepam, halazepam, lorazepam, midazolam, oxazepam, quazepam, temazepam and triazolam. In yet another variation, benzodiazepine prodrug is of the formula (VI) and a prodrug moiety of the formula (1A) benzodiazepine moiety is a moiety of any known benzodiazepine or derivatives thereof.

The prodrug may be, and any of the methods described herein may use, a prodrug of the formula (VII):

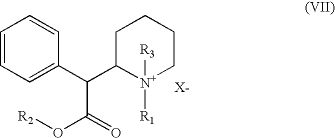

where R¹ is hydrogen or a substituted or unsubstituted C₁-C₅ alkyl; R² is a hydrogen or a substituted or unsubstituted alkyl; R³ is the prodrug moiety of formula (1A), (1B) or (2); X⁻ is a pharmaceutically acceptable anion, and any stereoisomer, salt, hydrate or solvate thereof.

The prodrug may be, and any of the methods described herein may use, a prodrug of the formula (VIII):

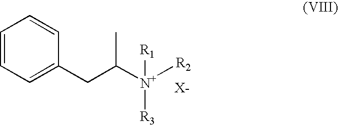

where R¹ is hydrogen or a substituted or unsubstituted C₁-C₅ alky; R² is a hydrogen or a substituted or unsubstituted alkyl or prodrug moiety of formula (1A), (1B) or (2); R³ is the prodrug moiety of formula (1A), (1B) or (2); X⁻ is a pharmaceutically acceptable anion, and any stereoisomer, salt, hydrate or solvate thereof. In case where both R² and R³ are a prodrug moiety of formula (1A), (1B) or (2), pharmaceutically acceptable anion X⁻ can be twice as much (e.g., 2 X⁻).

The prodrug may be, and any of the methods described herein may use, a prodrug of the formula (IX):

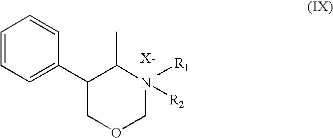

where R¹ is a hydrogen or a substituted or unsubstituted alkyl or prodrug moiety of formula (1A), (1B) or (2); R² is the prodrug moiety of formula (1A), (1B) or (2); X⁻ is a pharmaceutically acceptable anion, and any stereoisomer, salt, hydrate or solvate thereof. In case where both R¹ and R² are a prodrug moiety of formula (1A), (1B) or (2), pharmaceutically acceptable anion X⁻ can be twice as much (e.g., 2 X⁻).

Any of the prodrugs described herein may be formulated as a pharmaceutically acceptable composition e.g., by combining the prodrug with or dispensing the prodrug in a pharmaceutically acceptable carrier.

The described methods may use any of the prodrugs described herein. In one variation, the method is a method of delaying the onset of parent drug activity in an individual in need of parent drug therapy and where the method comprises administering to the individual an effective amount of a prodrug comprising a parent drug moiety and a prodrug moiety of the formula (1A), (1B) or (2), or any stereoisomer, salt, hydrate or solvate thereof, wherein the prodrug provides a slower onset of parent drug activity as compared to the parent drug. In one variation, the method is a method of delaying the onset of APD activity in an individual in need of APD therapy and where the method comprises administering to the individual an effective amount of a prodrug comprising an APD moiety and a prodrug moiety of the formula (1A), (1B) or (2), or any stereoisomer, salt, hydrate or solvate thereof, wherein the prodrug provides a slower onset of APD activity as compared to the parent APD. In other variations, the parent drug is an opioid, benzodiazepine, stimulant or anorexiant. In a particular variation, the parent drug is an opioid, benzodiazepine, stimulant or anorexiant where the opioid, benzodiazepine, stimulant or anorexiant is an APD. In another variation, the parent drug is an opioid, benzodiazepine, stimulant or anorexiant where the opioid, benzodiazepine, stimulant or anorexiant is not an APD.

In one variation, the method is a method of prolonging parent drug action in an individual in need of parent drug therapy and where the method comprises administering to an individual an effective amount of a prodrug comprising a parent drug moiety and a prodrug moiety of the formula (1A), (1B) or (2), or any stereoisomer, salt, hydrate or solvate thereof, wherein the prodrug provides prolonged parent drug action as compared to the parent drug. In one variation, the method is a method of prolonging opioid action in an individual in need of opioid therapy and where the method comprises administering to an individual an effective amount of an opioid prodrug comprising an opioid moiety and a prodrug moiety of the formula (1A), (1B) or (2), or any stereoisomer, salt, hydrate or solvate thereof, wherein the opioid prodrug provides prolonged opioid action as compared to the parent opioid. In other variations, the parent drug is an opioid, benzodiazepine, stimulant or anorexiant. In a particular variation, the parent drug is an opioid, benzodiazepine, stimulant or anorexiant where the opioid, benzodiazepine, stimulant or anorexiant is an APD. In another variation, the parent drug is an opioid, benzodiazepine, stimulant or anorexiant where the opioid, benzodiazepine, stimulant or anorexiant is not an APD. In yet one variation, the method is a method of prolonging methylphenidate, amphetamine or methamphetamine action in an individual in need of therapy by methylphenidate, amphetamine, or methamphetamine and where the method comprises administering to an individual an effective amount of methylphenidate, amphetamine, or methamphetamine prodrug comprising a methylphenidate, amphetamine, or methamphetamine moiety and a prodrug moiety of the formula (1A), (1B) or (2), or any stereoisomer, salt, hydrate or solvate thereof, wherein the methylphenidate, amphetamine, or methamphetamine prodrug provides prolonged methylphenidate, amphetamine, or methamphetamine action as compared to the methylphenidate, amphetamine, or methamphetamine itself.

In one variation, the method is a method of decreasing the abuse potential of an APD in an individual in need of APD therapy and where the method comprises administering to an individual an effective amount of a prodrug comprising an APD moiety and a prodrug moiety of the formula (1A), (1B) or (2), or any stereoisomer, salt, hydrate or solvate thereof, wherein the prodrug is less susceptible to abuse as compared to the parent APD. In one variation, the method is a method of decreasing the abuse potential of an opioid in an individual in need of opioid therapy and where the method comprises administering to an individual an effective amount of a prodrug comprising an opioid moiety and a prodrug moiety of the formula (1A), (1B) or (2), or any stereoisomer, salt, hydrate or solvate thereof, wherein the opioid prodrug is less susceptible to abuse as compared to the parent opioid. In another variation, the APD is a benzodiazepine, stimulant or anorexiant

In one variation, the method is a method of decreasing the abuse potential of methylphenidate, amphetamine, or methamphetamine in an individual in need of methylphenidate, amphetamine, or methamphetamine therapy and where the method comprises administering to an individual an effective amount of a prodrug comprising a methylphenidate, amphetamine, or methamphetamine moiety and a prodrug moiety of the formula (1A), (1B) or (2), or any stereoisomer, salt, hydrate or solvate thereof, wherein the methylphenidate, amphetamine, or methamphetamine prodrug is less susceptible to abuse as compared to the methylphenidate, amphetamine, or methamphetamine itself.

### BRIEF DESCRIPTION OF THE DRAWINGS

FIG. 1. HPLC analysis of N-Phosphonooxymethyl Levorphanol

FIG. 2. UV spectrum of N-Phosphonooxymethyl Levorphanol

FIG. 3. ¹H-NMR of N-Phosphonooxymethyl Levorphanol

FIG. 4. FT-IR of N-Phosphonooxymethyl Levorphanol

FIG. 5. Mass spectrum of N-Phosphonooxymethyl Levorphanol

FIG. 6. Chemical stability of N-Phosphonooxymethyl Levorphanol at pH 1.2

FIG. 7. Chemical stability of N-Phosphonooxymethyl Levorphanol at pH 6

FIG. 8. Chemical stability of N-Phosphonooxymethyl Levorphanol at pH 8

FIG. 9. Enzymatic stability of N-Phosphonooxymethyl Levorphanol

### DETAILED DESCRIPTION OF THE INVENTION

Prodrugs of the invention offer new therapies with fewer undesirable characteristics as compared to their parent drugs. For instance, a prodrug of an opioid agonist or antagonist that has reduced affinity for an opioid receptor as compared to its parent opioid and releases its parent opioid slowly, e.g., in the gastrointestinal tract, blood or at the site of administration, may be of significant value as a medicine. Release of a parent drug from a prodrug in the gastrointestinal tract can be mediated exclusively by enzymatic hydrolysis (i.e., hydrolysis by alkaline phosphatase, which is abundant in the large intestine) or it may occur as a combination of chemical and/or enzymatic hydrolysis. Release of a parent drug from a prodrug of the invention, such as an ADP prodrug, may lead to beneficial pharmacological effects, such as analgesia, anxiolysis, hypnosis, anticonvulsant, muscle relaxant, anorexia or CNS stimulation perhaps with reduced or negative side affects that can accompany administration of the parent drug itself.

APD prodrugs, such as prodrugs of opioid agonists, are believed to be less attractive to substance abusers or non-medical users of APDs who seek drugs that can provide rapid euphoria. That is, the latency of APD bioavailability as a function of, e.g., enzymatic or chemical release of the parent APD from the prodrug, prevents an APD prodrug from producing a fast onset of action as compared to administration of the parent APD. For instance, fast onset of action is known to increase the “street value” and “use again” value of diazepam compared to other benzodiazepines (see, O'Brien C. P. “Benzodiazepine Use, Abuse and Dependence”, J. Clin. Psychiatry 2005. 66[Suppl. 2]: 28-33). Other APDs, for instance, opioids are valued on the same grounds, i.e., quick onset of action, by those who would abuse them (see, Development and validation of an Opioid Attractiveness Scale: a novel measure of the attractiveness of opioid products to potential abusers, Harm Reduction Journal, 2006. 3:5). As the release of a parent APD, such as an opioid, from a prodrug will be delayed and gradual, the onset of euphoria attainment will likewise be slow and gradual, reducing the attractiveness of APD prodrugs, such as opioid prodrugs, to those who would consider non-medical usage of the drug.

Another advantage of slow or delayed release of a parent APD, such as an opioid agonist, from the prodrug (and thus controlled or delayed systemic absorption of the parent APD, such as an opioid) is that adverse events due to overdosing (e.g., respiratory depression) would develop slowly, if they develop at all, allowing the opportunity for quick intervention, e.g., with an opioid antagonist (e.g., naloxone). Administration of an APD prodrug, such as an opioid prodrug, should similarly increase the gastrointestinal tolerability of APDs, such as opioid analgesics, as the high local concentrations of APDs, such as opioid agonists, provided by current formulations will be reduced. That is, as enzymatic cleavage of the prodrug occurs, e.g., via alkaline phosphatase, the parent drug is slowly released in the lower portions of the gastrointestinal tract and absorbed, leaving a relatively low concentration of parent drug, such as opioid, in the lumen of the gut. The emesis, nausea and constipation produced by opioid analgesics are closely linked to high local concentrations in the gastrointestinal tract (see, Are peripheral opioid antagonists the solution to opioid side effects? Anesth. Analg. 2004. 98:116-122).

Prodrugs detailed throughout this disclosure are embraced by this invention, such as prodrugs detailed in the “Brief Summary of the Invention” and elsewhere. This invention also contemplates prodrugs of benzodiazepines, CNS stimulants, hypnotics, opioid antagonists, and anorexiants, such as but not limited to those with high abuse potential, such as phenmetrazine and levorphanol. One of the advantages of an opioid antagonist prodrug, for example, a methylnaltrexone prodrug, would be the use of opioid antagonists such as in relieving symptoms of constipation for which methylnaltrexone is very effective, but suffers the disadvantage of a narrow therapeutic index. Slow and localized release of opioid antagonists such as methylnaltrexone in the gastrointestinal tract would help circumvent this drawback by reducing the potential for quick and excessive systemic absorption (see, Are peripheral opioid antagonists the solution to opioid side effects? Anesth. Analg. 2004. 98:116-122). The slow enzymatic conversion of the prodrug to the parent opioid antagonist in the gastrointestinal tract can also provide delivery of the opioid antagonist at the intended site of action (lower intestine and colon), reducing the chance for therapeutically ineffective systemic absorption while retaining the ability to e.g., reverse opioid-induced analgesia. Similar advantages can be had where the prodrugs of the invention use other parent drugs, such as benzodiazepines, CNS stimulants, hypnotics and anorexiants.

Prodrugs that are inactive or less active at their biological site of action, such as at receptors, as compared to the parent drug, and methods of altering a parent drug to render it inactive at a biological site of action and using the same are embraced by this invention. For example, opioid prodrugs that are inactive or less active at opioid receptors as compared to the parent opioid and methods of altering a parent opioid to render it inactive at opioid receptors and using the same are embraced by this invention. Opioid prodrugs that are stable to chemical hydrolysis at various pHs similar to the pHs in the gastrointestinal system and yet releasable by an enzyme that is selectively active in the large intestine, methods of altering a parent opioid to render it stable to chemical hydrolysis at various pHs similar to the pHs in the gastrointestinal system and yet releasable by an enzyme that is selectively active in the large intestine, and methods of using the same are embraced by this invention. Methods of delaying the onset of opioid activity and/or prolonging opioid activity and/or decreasing the abuse potential of an opioid as compared to the parent opioid are also embraced by this invention. Methods for treating pain using the prodrugs or formulations herein are also described. In some variations, the pain is selected from the group consisting of pain associated with trauma (e.g., by surgery or otherwise), osteoarthritis, rheumatoid arthritis, lower back pain, fibromyalgia, postherpetic neuralgia, diabetic neuropathy, HIV-associated neuropathy and complex regional pain syndrome.

Among all benzodiazepines, diazepam and flurazepam have rapid onset of action upon oral administration, while they have long half lives and therefore require less frequent dosing. Their rapid onset of action raises their abuse potential, which adversely affects their selection for use. Prodrugs of these benzodiazepines contemplated by current invention would delay the onset of action, which may lead to their increased selection by physicians.

Parent drugs formulated for administration as an intravenous dosage form are candidates for solubility improvements by conversion to a prodrug. Improved water solubility of the prodrug means that high dose levels of compounds could be delivered in a small dose volume to patients without the fear that the administered drug would crystallize or precipitate at the site of administration and hence minimize or eliminate the risk of venous irritation and phlebitis. Formulations containing such prodrugs should be better tolerated, and may be safer, while still providing the necessary beneficial therapeutic effect(s). Even though this class of molecules will generally display improved water solubility, phosphate derivatives of parent drugs themselves would not be orally bioavailable because they would not be passively or actively absorbed from the gastrointestinal tract. It is widely recognized that compounds with phosphonic acid groups will not be either actively or passively absorbed from the gastrointestinal tract. Thus, the prodrugs of the invention will only be present in the gastrointestinal tract, and will either be metabolically activated or excreted. This results in a safety advantage in that individual will have at least decreased or no systemic exposure to a parent drug, such as an APD.

This invention also contemplates that a parent opioid, such as levorphanol, is available for systemic absorption following release from an opioid prodrug. Levorphanol or other opioids are released slowly over time, which mitigates the side effects associated with a rapid high dose of an opioid. These side effects include nausea, vomiting, dizziness, tiredness, somnolence and respiratory depression. The opioid prodrugs may display reduced or no affinity for the mu and/or other opioid receptors, and hence be essentially or completely pharmacologically inactive at the opioid receptors prior to bioconversion to the parent opioid. Without being bound by theory, it is believed that upon administration, the prodrug moiety of an opioid prodrug, such as a phosphonooxyalkyl group, is removed or cleaved in the large intestine, e.g., it may be hydrolyzed by alkaline phosphatase, to release free parent opioid in a controlled manner. Hence, a relatively delayed and sustained release profile is expected for an opioid prodrug as compared to a conventional immediate release profile following administration of the parent opioid, which would not only ensure a longer analgesia, but it will also minimize the risk of dose-dependent serious side effects such as respiratory depression.

This invention embraces prodrugs and methods of making and using the same wherein the parent drug moiety of the prodrug is obtainable from any parent drug, such as APDs, opioid agonists or antagonists of natural, semi-synthetic or synthetic origin, benzodiazepines, CNS stimulants, anorexiants and other drugs known for their abuse potential. Some exemplary opioids comprise the chemical structure shown in structure (A) below:

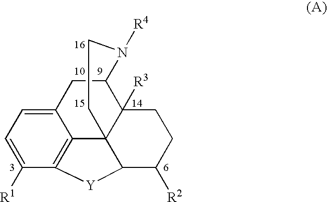

In structure (A), the portion of the structure shown in boldface represents a pharmacophore for opioid activity. Historically, derivatization of opioids has largely focused on chemical modification of positions R¹, R² and R³ of such compounds. The stringent structural requirement of these molecules as opioid agonists, antagonists, or mixed agonist-antagonists has rendered the tertiary amine a highly undesirable position for structure modification, as minor modification at this part of the molecule can lead to loss of activity. For example, in N-ethyl morphine when R⁴ is changed from ethyl to hydrogen, analgesic effects are reduced by 75%. N-substitution with a bulky group such as methylcyclopropyl is present in the case of many opioid antagonists such as nalbuphine, nalmefene, oxilorphan, naltrexone, cyclorphan—indicating that a change at nitrogen substituent can determine agonistic or antagonistic nature of the opioid. Therefore, historically, derivatization of the amine portion of the opioid nucleus of structure (A) has not been extensively attempted. However, we have found that derivatization of the amine of such opioids is attractive for use of such compounds as prodrugs, where decreased or no activity is desired of the prodrug and where the prodrug is capable of releasing the parent opioid, which has inherently higher biological activity when compared to the opioid prodrug.

This invention embraces opioid prodrugs and methods of making and using the same wherein the opioid moiety of the prodrug is obtainable from an opioid comprising the chemical structure as shown in structure (A) and where the prodrug moiety is connected to the opioid moiety via covalent attachment to the opioid nitrogen that contains R⁴ and where R¹, R², R³ and R⁴ may be as defined for formula (I) below. Preferably, such a prodrug does not bind to or exhibits only decreased or even no binding affinity for opioid receptors as compared to its parent opioid but does release a parent opioid that binds to or exhibits its inherent and higher binding affinity for opioid receptors as compared to the opioid prodrug. In one variation, the prodrug moiety is a methoxyphosphonic acid moiety. In another variation, the prodrug moiety is a ethoxyphosphonic acid moiety. The prodrug moiety may be the prodrug moiety of the formula (1A), (1B) or (2). In another variation, the physical and/or chemical properties of the prodrug are engineered to control the rate of hydrolysis and/or pharmacokinetics and/or pharmacodynamics by changing the nature of R¹, R², R³, and/or R⁴ groups of the opioid moiety of the prodrug where the opioid moiety is obtainable from an opioid with a structure shown in Structure A. The rate of hydrolysis and other pharmacokinetics properties can also be engineered by formulation chemistry.

This invention embraces prodrugs of any suitable parent drug, including but not limited to, benzodiazepines, CNS stimulants, opioids not represented by structure (A) and APDs, and methods of making and using the prodrugs. In one variation, the prodrug is a prodrug selected from the structures II-IX. In one aspect, the prodrug moiety is connected to the parent drug moiety via covalent attachment to a parent drug nitrogen. Preferably, such a prodrug does not exhibit inherent bioactivity of the parent drug at all or only exhibits a diminished bioactivity. However, upon enzymatic and/or chemical cleavage, the parent drug would be released to manifest its inherent bioactivity. Cleavage of the prodrug moiety introduces a delay in the onset of action of a parent drug. This delay would render prodrugs of parent drugs, such as APD prodrugs, less desirable for abusers seeking quick response. In one variation, the prodrug moiety is methoxyphosphonic acid. In another variation, the prodrug moiety is ethoxyphosphonic acid. The prodrug moiety may be of the formula (1A), (1B) or (2). In another variation, the physical and/or chemical properties of the prodrug are engineered to control the rate of hydrolysis and/or pharmacokinetics and/or pharmacodynamics by changing the nature of substituents of the parent drug moiety of the prodrug where the parent drug moiety is obtainable from any parent drug with a structure II-IX.

### DEFINITIONS

For use herein, unless clearly indicated otherwise, use of the term “Abuse-prone drug” or “APD” refers to any drug which has or is believed to have a potential for abusive use. The abusive use may be believed to lead to or be more likely to lead to a physical dependency or satisfy an existing physical dependency as compared to drugs in which no abuse potential is known or believed to exist in similar or different patient populations. Abusive use is generally compulsive in nature and lies outside the normal therapeutic utility of the drug.

For use herein, unless clearly indicated otherwise, use of the term “APD Prodrug” refers to a compound of the form APD MOIETY-PRODRUG MOIETY. An APD prodrug comprising a prodrug moiety of formula (1A), (1B) or (2) and an APD moiety, in which the prodrug moiety is bound to the APD moiety through a covalent bond. Derivatives, stereoisomers, salts, hydrates or solvates of an APD prodrug are embraced by the invention. For use herein, unless clearly indicated otherwise, use of the terms “a”, “an” and the like refers to one or more.

For use herein, unless clearly indicated otherwise, “an individual” as used herein intends a mammal, including but not limited to a human. The individual may be a human who is in need of parent drug therapy, such as opioid therapy. For example, the individual may be a human who exhibits one or more symptoms associated with acute or chronic pain. The individual may be a human who exhibits one or more symptoms associated with neuropathic pain such as a human who has been diagnosed with diabetic neuropathy, postherpetic neuralgia, HIV/AIDS, complex regional pain syndrome, trigeminal neuralgia, erythromelalgia or phantom pain or who has experienced traumatic injury to the nerves. The individual may be a human who exhibits one or more symptoms associated with inflammatory pain, such as a human who has been diagnosed with a chronic inflammatory condition such as osteoarthritis, rheumatoid arthritis or lower back pain. The individual may be a human who exhibits one or more symptoms associated with mixed inflammatory/neuropathic pain. The individual may be a human who exhibits one or more symptoms associated with pain due to traumatic injury or surgery. The individual may be a human who exhibits one or more symptoms associated with CNS injury or dysfunction. The individual may be a human who exhibits one or more symptoms associated with a sleep disorder. The individual may be a human who exhibits one or more symptoms associated with ADHD. The individual may be a human who has been diagnosed with or exhibits symptoms associated with an opioid-responsive condition. The individual may be a human who has been diagnosed with or exhibits symptoms associated with a benzodiazepine-responsive condition. The individual may be a human who has been diagnosed with or exhibits symptoms associated with a CNS drug-responsive condition. The individual may be a human who has been diagnosed with or exhibits symptoms associated with a stimulant-responsive condition. The individual may be a human who has been diagnosed with or exhibits symptoms associated with an anorexiant-responsive condition. The individual may be a human who has been diagnosed with or exhibits symptoms associated with an APD-responsive condition.

As used herein, an “effective dosage” or “effective amount” of a prodrug, drug, compound, or pharmaceutical composition is an amount that is expected to be or is sufficient to effect beneficial or desired results. For therapeutic use, beneficial or desired results include results such as suppressing or reducing the onset and/or development of a disease or condition or decreasing one or more symptoms resulting from a disease or condition that is responsive to parent drug therapy (biochemical, histological and/or behavioral), including increasing the quality of life of those suffering from a disease or condition responsive to parent drug therapy and/or decreasing the dose of the same or other medications, drugs, compounds or pharmaceutical compositions required to treat the disease or condition and/or decreasing or eliminating one or more side effects associated with a medication required to treat the individual's disease or condition. The disease or condition may be one that is believed to be responsive to opioid, benzodiazepine, stimulant, anorexiant, APD or CNS parent drugs. For example, for therapeutic use, beneficial or desired results include results such as suppressing or reducing the onset and/or development of pain or decreasing one or more symptoms resulting from a disease or condition that is responsive to opioid therapy (biochemical, histological and/or behavioral), including increasing the quality of life of those suffering from a disease or condition responsive to opioid therapy and/or decreasing the dose of the same or other medications, drugs, compounds or pharmaceutical compositions required to treat the disease or condition and/or decreasing or eliminating one or more side effects associated with a medication required to treat the individual's disease or condition. An effective dosage can be administered in one or more administrations. For purposes of this invention, an effective dosage of prodrug, drug, compound, or pharmaceutical composition is an amount sufficient to accomplish prophylactic or therapeutic treatment either directly or indirectly. As is understood in the clinical context, an effective dosage of a prodrug, drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, administration “in conjunction” includes simultaneous administration and/or administration at different times. Administration in conjunction also encompasses administration as a co-formulation or administration as separate compositions.

As used herein, “pharmaceutically acceptable carrier” includes any material which, when combined with an active ingredient, allows the ingredient to retain biological activity. Examples include, but are not limited to, any of the standard pharmaceutical carriers such as a phosphate buffered saline solution, water, emulsions such as oil/water emulsion, and various types of wetting agents. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

As used herein, “treatment” or “treating” is an approach for obtaining beneficial or desired results including clinical results. For purposes of this invention, beneficial or desired clinical results include, but are not limited to inhibiting, suppressing or reducing the onset and/or development and/or severity of a disease or condition or symptoms resulting from a disease or condition that is responsive to parent drug therapy, including increasing the quality of life of those suffering from a disease or condition responsive to parent drug therapy. The disease or condition may be one that is believed to be responsive to opioid, benzodiazepine, stimulant, anorexiant, APD or CNS parent drugs. For example, for purposes of this invention, beneficial or desired clinical results include, but are not limited to inhibiting, suppressing or reducing the onset and/or development and/or severity of pain or symptoms resulting from a disease or condition that is responsive to opioid therapy, including increasing the quality of life of those suffering from a disease or condition responsive to opioid therapy, or inhibiting, suppressing or reducing the onset and/or development and/or severity of psychological disorder or symptoms resulting from a disease or condition that is responsive to benzodiazepine therapy, including increasing the quality of life of those suffering from a disease or condition responsive to benzodiazepine therapy, or inhibiting, suppressing or reducing the onset and/or development and/or severity of ADHD or symptoms resulting from a disease or condition that is responsive to therapy by CNS stimulants, including increasing the quality of life of those suffering from a disease or condition responsive to therapy by CNS stimulant, or by inhibiting, suppressing or reducing the onset and/or development and/or severity of symptoms resulting from a disease or condition that is responsive to therapy by phenmetrazine or phendimetrazine, including increasing the quality of life of those suffering from a disease or condition responsive to therapy by phenmetrazine or phendimetrazine, and/or decreasing the dose of other medications required to treat the disease or condition and/or decreasing or eliminating one or more side effects associated with a medication required to treat the individual's disease or condition. In one variation, the methods and compositions, in particular the opioid prodrugs, of the present invention are useful for the treatment of pain of any etiology, including acute and chronic pain, any pain with an inflammatory component, and any pain in which an opioid analgesic is usually prescribed. Examples of pain include post-surgical pain, post-operative pain (including dental pain), migraine, headache and trigeminal neuralgia, pain associated with burn, wound or kidney stone, pain associated with trauma (including traumatic head injury), neuropathic pain (e.g., peripheral neuropathy and post-herpetic neuralgia), pain associated with musculo-skeletal disorders such as rheumatoid arthritis, osteoarthritis, cystitis, pancreatitis, inflammatory bowel disease, ankylosing spondylitis, sero-negative (non-rheumatoid) arthropathies, non-articular rheumatism and peri-articular disorders, and pain associated with cancer (including “break-through pain” and pain associated with terminal cancer). Examples of pain with an inflammatory component (in addition to some of those described above) include rheumatic pain, pain associated with mucositis, and dysmenorrhea. In some variations, the methods and compositions of the present invention are used for treatment or prevention of post-surgical pain and cancer pain. In some variations, the methods and compositions of the present invention are used for treatment or prevention of pain that is selected from the group consisting of pain associated with surgery, trauma, osteoarthritis, rheumatoid arthritis, lower back pain, fibromyalgia, postherpetic neuralgia, diabetic neuropathy, HIV-associated neuropathy and complex regional pain syndrome. In another variation, the methods and compositions of the present invention, in particular the CNS prodrugs, are useful for the treatment of psychic disorder of any etiology, including acute and chronic in nature, any psychic disorder in which a benzodiazepine or a CNS stimulant is usually prescribed. Examples of psychic disorder include transient or short term insomnia, acute stress reactions, episodic anxiety, generalized anxiety, adjustment disorder, severe panic disorder, agoraphobia, epilepsy, some motor disorders, acute psychoses, depression, muscle spasms and seizures, dizziness, malaise, headache, pallor, ADHD, and compulsive overeating.

As used herein, “opioid” or “opioid analgesic” refers in a generic sense to all drugs, natural, synthetic, or semi-synthetic that are capable of acting at an opioid receptor, such as may be determined by in vitro binding assays known by those of skill in the art. In accordance with the present invention, opioids include agents that can act on one or more opioid receptors, such as mu, delta, and kappa, to which morphine, the enkephalins, and the dynorphins, respectively, bind, and subtypes thereof. Pharmacologically these compounds can have diverse activities, thus some are strong agonists at the opioid receptors (e.g., morphine); others are moderate to mild agonists (e.g., codeine); still others exhibit mixed agonist-antagonist activity (e.g., nalbuphine); and yet others are partial agonists (e.g., nalorphine). In some variations, the opioid is an opioid antagonist such as naloxone. In other variations, the opioid is an opioid agonist.

As used herein, “opioid prodrug” refers to a compound of the form OPIOID MOIETY-PRODRUG MOIETY. The opioid prodrug is converted to or releases the parent opioid within the body through enzymatic or non-enzymatic reactions (e.g., chemical hydrolysis). An opioid prodrug may be made by any method, such as by linear synthesis or conjugation of a prodrug moiety to an opioid moiety. For instance, a parent opioid may be modified by the covalent attachment of a prodrug moiety to provide the opioid prodrug. The parent opioid is generally obtainable by removal of the prodrug moiety, e.g., by hydrolysis or enzymatic cleavage of the prodrug moiety.

As used herein, “parent opioid” refers to an opioid that does not contain a prodrug moiety. For instance, the parent opioid of the levorphanol prodrug (compound 7) of Example 1 is levorphanol.

As used herein, “parent drug” refers to a drug that does not contain a prodrug moiety.

As used herein, “benzodiazepines” refers in a generic sense to all benzodiazepines natural, synthetic, or semi-synthetic that are known by those of skill in the art. Pharmacologically these compounds can have diverse activities such as hypnotics, anxiolytics, anticonvulsants, myorelaxants, and amnesics.

As used herein, “benzodiazepine prodrug” refers to a compound of the form BENZODIAZEPINE MOIETY-PRODRUG MOIETY. The benzodiazepine prodrug is converted to or releases the parent benzodiazepine within the body through enzymatic or non-enzymatic reactions (e.g., chemical hydrolysis). A benzodiazepine prodrug may be made by any method, such as by linear synthesis or conjugation of a prodrug moiety to a benzodiazepine moiety. For instance, a parent benzodiazepine may be modified by the covalent attachment of a prodrug moiety to provide the benzodiazepine prodrug. The parent benzodiazepine is generally obtainable by removal of the prodrug moiety, e.g., by hydrolysis or enzymatic cleavage of the prodrug moiety.

As used herein, “parent benzodiazepine” refers to a benzodiazepine that does not contain a prodrug moiety. For instance, the parent benzodiazepine of the diazepam prodrug is diazepam.

As used herein, “CNS drugs” are drugs that affect the central nervous system.

As used herein, “CNS stimulants” refers in a generic sense to all CNS stimulants, natural, synthetic, or semi-synthetic that are known by those of skill in the art. By way of example, but not limitation, such CNS stimulants include methylphenidate, dexmethylphenidate, amphetamine, and methamphetamine.

As used herein, “CNS stimulant prodrug” refers to a compound of the form CNS STIMULANT MOIETY-PRODRUG MOIETY. The CNS stimulant prodrug is converted to or releases the parent CNS stimulant within the body through enzymatic or non-enzymatic reactions (e.g., chemical hydrolysis). A CNS stimulant prodrug may be made by any method, such as by linear synthesis or conjugation of a prodrug moiety to a CNS stimulant moiety. For instance, a parent CNS stimulant may be modified by the covalent attachment of a prodrug moiety to provide the CNS stimulant prodrug. The parent CNS stimulant is generally obtainable by removal of the prodrug moiety, e.g., by hydrolysis or enzymatic cleavage of the prodrug moiety.

As used herein, “parent CNS stimulant” refers to a CNS stimulant that does not contain a prodrug moiety. For instance, the parent CNS stimulant of the methylphenidate prodrug is methylphenidate.

As used herein, “anorexiant” refers in a generic sense to all anorexiants, natural, synthetic, or semi-synthetic that are known by those of skill in the art. By way of example, but not limitation, such anorexiants include phenmetrazine and phendimetrazine.

As used herein, “anorexiant prodrug” refers to a compound of the form ANREXIANT MOIETY-PRODRUG MOIETY. The anorexiant prodrug is converted to or releases the parent anorexiant within the body through enzymatic or non-enzymatic reactions (e.g., chemical hydrolysis). An anorexiant prodrug may be made by any method, such as by linear synthesis or conjugation of a prodrug moiety to an anorexiant moiety. For instance, a parent anorexiant may be modified by the covalent attachment of a prodrug moiety to provide the anorexiant prodrug. The parent anorexiant is generally obtainable by removal of the prodrug moiety, e.g., by hydrolysis or enzymatic cleavage of the prodrug moiety.

As used herein, “parent anorexiant” refers to a anorexiant that does not contain a prodrug moiety. For instance, the parent anorexiant of the phenmetrazine prodrug is phenmetrazine.

As used herein, “opioid moiety” refers to the residue or radical of a parent opioid that is present in the opioid prodrug. For instance, the opioid moiety of the levorphanol prodrug (compound 7) of Example 1 is the portion of the levorphanol prodrug that is derivable from levorphanol:

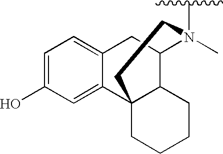

As used herein, “delaying the onset” or “delayed onset” refers to the increased time to onset of action provided by a prodrug as compared to administration of the same amount of parent drug within the same time period through the same route of administration. For example, an opioid prodrug preferably has no or reduced affinity for opioid receptors as compared to the parent opioid and releases the parent opioid slowly either in the gastrointestinal tract, blood or at the site of administration. Release of parent opioid in the gastrointestinal tract can be mediated exclusively by enzymatic hydrolysis (i.e., hydrolysis by alkaline phosphatase, which is abundant in the large intestine) or it may be a combination of chemical and enzymatic hydrolysis or by other chemical reactions. The slow release of parent opioid from the prodrug should result in delayed systemic exposure to the parent opioid as compared to administration of the same amount of parent opioid to an individual. Similar results may be obtained by other prodrugs of the invention.

As used herein, “prolonging activity” or “prolonged activity” refers to the sustained action provided by a prodrug by virtue of the time required to release or otherwise generate the parent drug from the prodrug. For example, administration of an opioid prodrug may result in sustained release of the parent opioid as compared to administration of the same amount of parent opioid over the same time period through the same route of administration. “Sustained release” refers to release of the parent drug, such as an opioid, at a rate such that the blood concentration of the parent drug, such as an opioid or a metabolite thereof, in an individual is maintained at or within the therapeutic range (e.g., above the minimum effective analgesic concentration but below toxic levels) for an extended duration. The extended duration in this context intends any time greater than the time that the same amount of corresponding parent opioid, administered as the parent opioid and not as an opioid prodrug, results in a parent opioid (or metabolite thereof) blood concentration within the therapeutic range.

As used herein, “decrease the abuse potential” or “decreased abuse potential” refers to the reduced potential of an APD prodrug for improper administration as compared to its parent APD and where the APD prodrug is still capable of delivering a therapeutically effective dose of the parent APD when administered as directed. The overall abuse potential of an APD or prodrug thereof is not established by any one single factor. Instead, there is a composite of factors, including, the capacity of the drug to produce the kind of physical dependence in which drug withdrawal causes sufficient distress to bring about drug-seeking behavior; the ability to suppress withdrawal symptoms caused by withdrawal from other agents; the degree to which it induces euphoria similar to that produced by morphine and other opioids; the swiftness of induction of eurphoria; the patterns of toxicity that occur when the drug is dosed above its normal therapeutic range; and physical characteristics of the drugs such as water solubility. One or more factors such as latency of action and/or sustained release of a parent APD, such as an opioid, should render the APD prodrug less attractive for substance abuse as compared to the parent APD as it will not rapidly induce euphoria and/or will not provide drug blood levels of parent APD above the therapeutic range for sustained periods of time and/or will not require multiple bolus doses in order to maintain therapeutic drug blood levels of the parent opioid or a metabolite thereof. The abuse potential of an APD prodrug may be compared to that of its parent APD by methods known in the art, including without limitation patient questionnaires such as those described in U.S. Patent Publication No. 2004008656 and in Jasinski D R., “Assessment of the Abuse Potential of Morphine-Like Drugs (methods used in man).” In: Drug Addiction I (Martin, W. R., ed.), 1997:197-258. Springer-Verlag, New York and Preson K L, Jasinski D R, Testa M. “Abuse Potential and Pharmacological Comparison of Tramadol and Morphine.” Drug and Alcohol Dependence 1991; 27:7-17; each of which is hereby incorporated by reference. A comparison of the attractiveness of the opioid prodrugs with existing opioid analgesics can also be made using a validated clinical instrument called the Opioid Attractiveness Scale (see, Development and validation of an Opioid Attractiveness Scale: a novel measure of the attractiveness of opioid products to potential abusers, Harm Reduction Journal, 2006. 3:5). The relative attractiveness of an opioid prodrug and its potential for abuse can also be predicted on the basis of in vivo studies involving rodents, pigs, dogs or non-human primates, such as drug discrimination, self administration and dependence potential assays. For example, see: Colpaert F C and Janssen P A. OR discrimination: a new drug discrimination method. Eur J. Pharmacol. 1982 Feb. 19; 78(1):141-144; or Lyness W H, Smith F L, Heavner J E, Iacono C U, Garvin R D. Morphine self-administration in the rat during adjuvant-induced arthritis. Life Sci. 1989; 45(23):2217-2224.

“Alkyl” refers to linear, branched or cyclic hydrocarbon structures preferably having from 1 to 20 carbon atoms (a “C₁-C₂₀ alkyl”) and more preferably 1 to 10 carbon atoms or 1 to 6 carbon atoms. This term is exemplified by groups such as methyl, t-butyl, n-heptyl, octyl, cyclobutylmethyl, cyclopropylmethyl and the like. “Unsubstituted alkyl” refers to an alkyl group that is not substituted with any additional substituents. When an alkyl residue having a specific number of carbons is named, all geometric isomers having that number of carbons are intended to be encompassed; thus, for example, “butyl” is meant to include n-butyl, sec-butyl, isobutyl and t-butyl.

“Substituted alkyl” refers to an alkyl group, preferably of from 1 to 10 carbon atoms, having from 1 to 5 substituents, including but not limited to, groups such as halogen, alkoxy, acyl, acylamino, acyloxy, amino, hydroxyl, mercapto, carboxyl, aryl, cyano, nitro and the like. For instance, an alkaryl group (alkyl-aryl) is a substituted alkyl and includes moieties such as propylbenzene where the moiety is attached to the parent structure via the aryl or the alkyl portion, most preferably via the alkyl portion of the substituent.

“Alkenyl” refers to linear, branched or cyclic hydrocarbon structures preferably having from 2 to 20 carbon atoms (a “C₁-C₂₀ alkenyl”) and more preferably 2 to 10 carbon atoms or 2 to 6 carbon atoms and having at least 1 and preferably from 1-2 sites of alkenyl unsaturation. “Unsubstituted alkenyl” refers to an alkenyl group that is not substituted with any additional substituents. When an alkenyl residue having a specific number of carbons is named, all geometric isomers having that number of carbons are intended to be encompassed. This term is exemplified by groups such as propen-3-yl (—CH₂—CH═CH₂), 3-methyl-but-2-enyl and (═CH₂). The group represented by ═CH₂ indicates connectivity from, e.g., an sp2 hybridized carbon atom of a parent structure to CH₂ via a double bond.

“Substituted alkenyl” refers to an alkenyl group, preferably a C₂-C₁₀ alkenyl, having from 1 to 5 substituents, including but not limited to, substituents such as halogen, alkoxy, acyl, acylamino, acyloxy, amino, hydroxyl, mercapto, carboxyl, aryl, cyano, nitro and the like.

“Alkoxy” refers to the group “alkyl-O—” which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Alkoxyalkyl” refers to the group “alkyl-O-alkyl-” which includes, by way of example, methoxy methyl and the like.

“Alkanoate” refers to “alkyl-C(═O)—O—” which includes, by way of example, ethanoate and pentanoate. “Alkyl-Alkanoate” refers to “-alkyl-O—C(═O)alkyl” such as in —CH(CH₂CH₃)—O—C(═O)—CH₃.

“Carbonylalkyl” refers to —C(═O)-alkyl, which includes, by way of example, —C(═O)—CH₂CH₃.

“Alkoxyphosphonic acid” refers to “alkyl —O—P(═O)(OH)₂” or when referred to or implied as a moiety attached to a parent structure, the radical “-alkyl-O—P(═O)(OH)₂” such that the alkoxyphosphonic acid is attached to a parent structure via the alkyl moiety. This term is exemplified by groups such as methoxyphosphonic acid and ethoxyphosphonic acid and their radicals —CH₂—O—P(═O)(OH)₂—CH(CH₃)OP(O)(OH)₂ and —CH₂CH₂—O—P(═O)(OH)₂.

“Alkylcarbonylalkoxy” refers to alkyl-C(═O)—O-alkyl. In one variation, the alkylcarbonylalkoxy refers to a moiety C₁-C₄ alkyl-C(═O)—O—C₁-C₆ alkyl. An exemplary alkylcarbonylalkoxy is —CH₂CH₂C(═O)OCH₃.

### Compounds for Use in the Methods, Formulations and Kits

Parent drugs may be modified in accordance with this invention to include a physiologically and biocompatible removable prodrug moiety which is removable in vivo to provide for the parent drug, a pharmaceutically acceptable salt thereof or a biologically active metabolite thereof. Any parent drug with a tertiary or secondary amine is suitable for use in the methods described herein. Administration of the prodrug preferably results in one or more of: delayed onset of parent drug activity, prolonged parent drug activity and/or decreased abuse potential as compared to administration of the parent drug itself. The invention embraces prodrugs of the form PARENT DRUG MOIETY-(CH₂)ₙ—O—P(═O)(OH)₂, where n is an integer from 1 to 10. Also embraced are prodrugs of the form PARENT DRUG MOIETY-PRODRUG MOIETY (1A), PARENT DRUG MOIETY-PRODRUG MOIETY (1B) and PARENT DRUG MOIETY-PRODRUG MOIETY (2). In one variation, when n=1, the parent drug moiety is not a moiety of an parent drug selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In another variation, when n=1, the parent drug moiety is a moiety of an parent drug selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation when the parent drug is an opioid, the opioid moiety is not a moiety of the opioid levorphanol. In one variation, the opioid moiety is a moiety of the opioid levorphanol.

Particular prodrugs that may be used in the methods, formulations and kits herein include without limitation levorphanol ethylphosphate, oxycodone ethylphosphate, hydrocodone ethylphosphate, oxymorphone ethylphosphate, codeine ethylphosphate, fentanyl ethylphosphate, methadone ethylphosphate, buprenorphine ethylphosphate, DiPOA ((8-3,3-diphenyl-propyl)-4-oxo-1-phenyl-1,3,8-triaza-spiro[4.5]dec-3-yl-acetic acid) methylphosphate, DiPOA ethylphosphate, amphetamine methylphosphate, amphetamine ethylphosphate, methamphetamine methylphosphate, methamphetamine ethylphosphate, methylphenidate methylphosphate, methylphenidate ethylphosphate, dexmethylphenidate methylphosphate, dexmethylphenidate ethylphosphate, phenmetrazine methylphosphate, phenmetrazine ethylphosphate, phendimetrazine methylphosphate, phendimetrazine ethylphosphate, diazepam methylphosphate, diazepam ethylphosphate, carfentanil methylphosphate, carfentanil ethylphosphate, remifentanil methylphosphate, remifentanil ethylphosphate, levo-alphacetylmethadol methylphosphate, levo-alphacetylmethadol ethylphosphate, ethylmorphine methylphosphate, ethylmorphine ethylphosphate and clonazepam ethylphosphate. In one variation, the opioid prodrug is levorphanol methylphosphate. In another variation, the APD prodrug is methylphenidate methylphosphate. In another variation, the APD prodrug is amphetamine methylphosphate. In another variation, the APD prodrug is methamphetamine methylphosphate. In still another variation, the APD prodrug is remifentanil methylphosphate. In yet another variation, the APD prodrug is carfentanil methylphosphate.

In one variation, the prodrug comprises a parent drug moiety and a prodrug moiety of the formula:

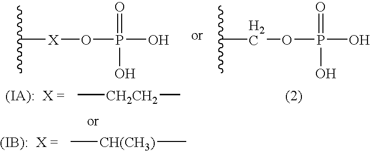

For prodrugs comprising the prodrug moiety (1A) or (1B), there is the additional advantage over opioid prodrugs comprising the prodrug moiety (2) of decreased toxicity or potential for adverse biological effects. That is, when a parent opioid is released from a prodrug comprising the prodrug moiety (2), the prodrug may ultimately degrade to form formic acid. However, prodrugs comprising the prodrug moiety (1A) or (1B) will not degrade to formic acid and may be desired for their improved safety and tolerance.

In one variation, when the prodrug moiety is of the formula (2), the parent drug moiety is not a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation, when the prodrug moiety is of the formula (2), the parent drug moiety is a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation, the opioid moiety is not a moiety of the opioid levorphanol. In one variation, the opioid moiety is a moiety of the opioid levorphanol.

In one variation, the opioid prodrug is of the formula (I):

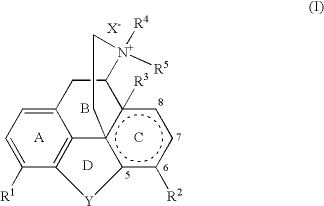

wherein R¹ is selected from the group consisting of hydrogen, C₁-C₁₀ alkanoate, hydroxyl and a substituted or unsubstituted C₁-C₁₀ alkyl and substituted or unsubstituted C₁-C₁₀ alkoxy; R² is selected from the group consisting of hydrogen, ═O (meaning that the hydrogen on carbon 6 is not present and R² results in a double bond from an sp2 hybridized carbon at position 6 to oxygen), hydroxyl, a substituted or unsubstituted C₁-C₁₀ alkyl, a substituted or unsubstituted C₂-C₁₀ alkenyl (including without limitation when the hydrogen on carbon 6 is not present and R² results in a double bond from an sp2 hybridized carbon at position 6 to a CH₂ group (e.g., ═CH₂)) and a substituted or unsubstituted C₁-C₁₀ alkoxy; R³ is selected from the group consisting of hydrogen, hydroxyl, C₁-C₁₀ alkanoate, a substituted or unsubstituted C₁-C₁₀ alkyl and a substituted or unsubstituted C₁-C₁₀ alkoxy; R⁴ is selected from a group consisting of hydrogen, C₁-C₁₀ alkanoate, a substituted or unsubstituted C₁-C₁₀ alkyl, a substituted or unsubstituted C₂-C₁₀ alkenyl and a substituted or unsubstituted C₁-C₁₀ alkoxy; R⁵ is the prodrug moiety (1A) or (1B) or (2); Y is null (indicating that ring D is not present) or is selected from O and S; ring C has zero, one or two double bonds; X⁻ is pharmaceutical acceptable anion; or any stereoisomer, salt, hydrate or solvate thereof. In one variation, the opioid prodrug is of the formula (I) provided that when R⁵ is the prodrug moiety (2), the opioid moiety is not a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In one variation, the opioid moiety of the opioid prodrug depicted by formula (I) is derivable from the parent opioid levorphanol or oxycodone or hydrocodone or oxymorphone or hydromorphone or codeine or morphine or naltrexone or naloxone or nalmefene or nalorphine or nalbuphine or cyclorphan or oxilorphan or levallorphan. In another variation, opioid prodrug is of the formula (I) and a prodrug moiety of the formula (1B) provided that opioid moiety is not a moiety of an opioid selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam. In yet another variation, opioid prodrug is of the formula (I) and a prodrug moiety of the formula (1A) opioid moiety is a moiety of any known opioid or derivatives thereof.

In one variation the opioid prodrug is of the formula (I) wherein R¹ is hydroxyl or a C₁-C₆ alkoxy; R² is hydrogen, hydroxyl, a C₂-C₆ alkenyl, or ═O; R³ is hydrogen or hydroxyl, R⁴ is a C₁-C₆ alkyl; Y is null or oxygen and C⁷ and C⁸ are optionally connected by a double bond. In one variation the opioid prodrug is of the formula (I wherein R¹ is hydroxyl or methoxy; R² is hydrogen, hydroxyl, ═CH₂, or ═O; R³ is hydrogen or hydroxyl, R⁴ is methyl, cyclopropylmethyl, propen-3-yl or cyclobutylmethyl; Y is null or oxygen and C⁷ and C⁸ are optionally connected by a double bond. In one embodiment, C⁷ and C⁸ of any variation herein are connected by a double bond. In one embodiment, ring C of any variation of formula (I) contains zero double bonds.

In one variation the opioid prodrug is of the formula (I) wherein R¹ is selected from the group consisting of hydrogen, alkanoate, hydroxyl, alkyl and alkoxy; R² is selected from the group consisting of hydrogen, ═O, hydroxyl, alkyl, alkenyl and alkoxy; R³ is selected from the group consisting of hydrogen, hydroxyl, alkanoate, alkyl and alkoxy; R⁴ is selected from the group consisting of hydrogen, alkanoate, alkyl, alkenyl and alkoxy; and Y is null or oxygen.

Any of the alkyl, alkenyl or alkoxy substituents listed above for formula (I) or below for formulae (II) through (IX) may in one variation be replaced with a substituted alkyl, a substituted alkenyl or a substituted alkoxy substituent, respectively.

In one variation, the opioid prodrug is of the formula (II):

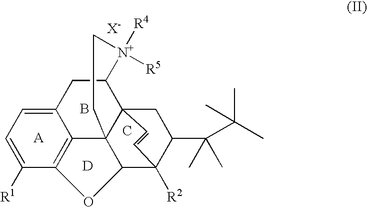

where R¹ is selected from the group consisting of hydrogen, hydroxyl, a substituted or unsubstituted C₁-C₁₀ alkyl and a substituted or unsubstituted C₁-C₁₀ alkoxy; R² is selected from the group consisting of hydrogen, hydroxyl, a substituted or unsubstituted C₁-C₁₀ alkyl, a substituted or unsubstituted C₂-C₁₀ alkenyl and a substituted or unsubstituted C₁-C₁₀ alkoxy; R⁴ is selected from a group consisting of hydrogen, a substituted or unsubstituted C₁-C₁₀ alkyl and a substituted or unsubstituted C₁-C₁₀ alkoxy; R⁵ is methoxyphosphonic acid or ethoxyphosphonic acid; ring C has zero or one double bond; and X⁻ is pharmaceutical acceptable anion, or any stereoisomer, salt, hydrate or solvate thereof. In one variation, the opioid moiety of the opioid prodrug is derivable from the parent opioid buprenorphine. In one variation, R⁵ is a prodrug moiety of formula (1A), (1B) or (2).

In one variation, the opioid prodrug is of the formula (III):

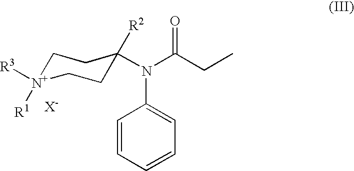

wherein R¹ is selected from the group consisting of hydroxyl, propylbenzene, ethylbenzene, 2-propylthiophene, methyl butyrate, 1-ethyl-4-ethyl-1H-tetrazol-5(4H)-one and 1-ethyl-4-propyl-1H-tetrazol-5(4H)-one, a substituted or unsubstituted C₁-C₁₀ alkyl and a substituted or unsubstituted C₁-C₁₀ alkoxy, alkylcarbonylalkoxy; R² is selected from the group consisting of hydrogen, hydroxyl, a substituted or unsubstituted C₁-C₁₀ alkyl and C₁-C₁₀ alkoxy, C₁-C₁₀ alkanoate, C₂-C₁₀ alkoxyalkyl; R³ is selected from a group consisting of methoxyphosphonic acid and ethoxyphosphonic acid; X⁻ is a pharmaceutically acceptable anion, and any stereoisomer, salt, hydrate or solvate thereof. In one variation, R³ is a prodrug moiety of formula (1A), (1B) or (2). In one variation, the opioid moiety of the opioid prodrug is derivable from the parent opioid fentanyl, sufentanil, alfentanil, carfentanil, or remifentanil.

In one variation, the opioid prodrug is of the formula (III) wherein R¹ is propylbenzene, 2-propylthiophene or 1-ethyl-4-ethyl-1H-tetrazol-5(4H)-one; R² is hydrogen, methoxy methyl or methyl formoate; and R³ is ethoxyphosphonic acid. In another variation, the opioid prodrug is of the formula (III) wherein R¹ is propylbenzene, 2-propylthiophene or 1-ethyl-4-ethyl-1H-tetrazol-5(4H)-one; R² is hydrogen, methoxy methyl or methyl formoate; and R³ is methoxyphosphonic acid. In one variation, R³ is a prodrug moiety of formula (1A), (1B) or (2).

In one variation, the opioid prodrug is of the formula (IV):

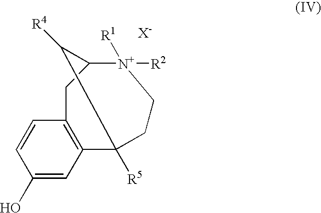

where R⁴ and R⁵ are independently alkyl; R² is methoxyphosphonic acid or ethoxyphosphonic acid; R¹ is alkaryl or alkenyl and X⁻ is a pharmaceutically acceptable anion, and any stereoisomer, salt, hydrate or solvate thereof. In one variation, R² is a prodrug moiety of formula (1A), (1B) or (2). In one variation, the prodrug is of the formula (IV) where R⁴ and R⁵ are independently selected a substituted or unsubstituted C₁-C₁₀ alkyl; R² is methoxyphosphonic acid or ethoxyphosphonic acid; R¹ is a C₁-C₁₀ alkaryl or a C₂-C₁₀ alkenyl and X⁻ is a pharmaceutically acceptable anion, and any stereoisomer, salt, hydrate or solvate thereof. In one variation, the prodrug is of the formula (IV) where R⁴ and R⁵ are independently selected a substituted or unsubstituted C₁-C₅ alkyl; R² is methoxyphosphonic acid or ethoxyphosphonic acid; R¹ is —CH₂)ₙ-phenyl where n is selected from 1 to 5 or a C₂-C₁₀ alkenyl and X⁻ is a pharmaceutically acceptable anion, and any stereoisomer, salt, hydrate or solvate thereof. In one variation, the opioid moiety of the opioid prodrug is derivable from the parent opioid pentazocine or phenazocine.

In one variation, the opioid prodrug is of the formula (V):

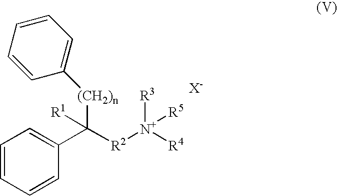

wherein R¹ is alkyl-alkanoate, an alkanoate or a carbonylalkyl; R², R³ and R⁴ are independently a substituted or unsubstituted alkyl; R⁵ is selected from a group consisting of methoxyphosphonic acid and ethoxyphosphonic acid; n is an integer from 1 to 10 and X⁻ is a pharmaceutically acceptable anion, and any stereoisomer, salt, hydrate or solvate thereof. In one variation, R⁵ is a prodrug moiety of formula (1A), (1B) or (2). In one variation, the prodrug is of the formula (V) where R¹ is a C₁-C₁₀ alkanoate or a C₁-C₁₀ carbonylalkyl; R², R³ and R⁴ are independently a substituted or unsubstituted C₁-C₁₀ alkyl; R⁵ is selected from a group consisting of methoxyphosphonic acid and ethoxyphosphonic acid; n is an integer from 1 to 10 and X⁻ is a pharmaceutically acceptable anion, and any stereoisomer, salt, hydrate or solvate thereof. In one variation, the prodrug is of the formula (V) where R¹ is propanoate or propionyl; R², R³ and R⁴ are independently a substituted or unsubstituted C₁-C₅ alkyl; R⁵ is selected from a group consisting of methoxyphosphonic acid and ethoxyphosphonic acid; n is an integer from 1 to 5 and X⁻ is a pharmaceutically acceptable anion, and any stereoisomer, salt, hydrate or solvate thereof. In one variation, the opioid moiety of the opioid prodrug is derivable from the parent opioid propoxyphene or methadone.

The prodrug may be, and any of the methods described herein may use, a benzodiazepine prodrug of the formula (VI):

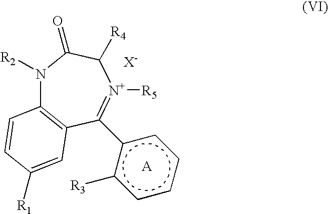

where R¹ is a halogen, nitro group, —NR₂, —NHR, —NH₂, —SO₃H, —CF₃, —C(O)Cl, —C(O)OH, —C(O)R, —C(O)OR, —C(O)H or alkyl or hydrogen where each R is independently a substituted or unsubstituted C₁-C₅ alkyl; R² is a hydrogen or a substituted or unsubstituted alkyl; R³ is hydrogen, halogen or a substituted or unsubstituted C₁-C₅ alkyl and R⁴ is a hydrogen, nitro group, hydroxyl, or ═O; Ring A aromatic or is non-aromatic but has one or two double bonds; R⁵ is the prodrug moiety (1A), (1B) or (2); X⁻ is a pharmaceutically acceptable anion, and any stereoisomer, salt, hydrate or solvate thereof. The benzodiazepine prodrug may be and any of the methods described herein may use a prodrug of the formula (VI) where R⁴ is chlorine or nitro group; R² is hydrogen or methyl, R³ is hydrogen or fluorine or chlorine and R⁴ is hydrogen, nitro group or ═O; R⁵ is the prodrug moiety (1A), (1B) or (2); X⁻ is a pharmaceutically acceptable anion, and any stereoisomer, salt, hydrate or solvate thereof.

In one variation, the benzodiazepine prodrug is of the formula (VI) provided that when the prodrug moiety is of the formula (2), the benzodiazepine moiety is not a moiety of a benzodiazepine selected from the group consisting of alprazolam, clorazepate, clonazepam, estazolam, flurazepam, halazepam, lorazepam, midazolam, oxazepam, quazepam, temazepam and triazolam. In another variation, benzodiazepine prodrug is of the formula (VI) and a prodrug moiety of the formula (1B) provided that benzodiazepine moiety is not a moiety of an benzodiazepine selected from the group consisting of alprazolam, clorazepate, clonazepam, estazolam, flurazepam, halazepam, lorazepam, midazolam, oxazepam, quazepam, temazepam and triazolam. In yet another variation, benzodiazepine prodrug is of the formula (VI) and a prodrug moiety of the formula (1A) benzodiazepine moiety is a moiety of any known benzodiazepine or derivatives thereof.

The prodrug may be, and any of the methods described herein may use, a prodrug of the formula (VII):

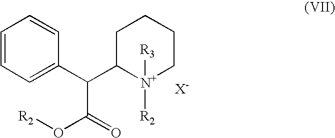

where R¹ is hydrogen or a substituted or unsubstituted C₁-C₅ alkyl; R² is a hydrogen or a substituted or unsubstituted alkyl; R³ is the prodrug moiety of formula (1A), (1B) or (2); X⁻ is a pharmaceutically acceptable anion, and any stereoisomer, salt, hydrate or solvate thereof.

The prodrug may be, and any of the methods described herein may use, a prodrug of the formula (VIII):

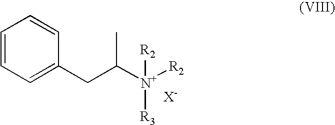

where R¹ is hydrogen or a substituted or unsubstituted C₁-C₅ alky; R² is a hydrogen or a substituted or unsubstituted alkyl or prodrug moiety of formula (1A), (1B) or (2); R³ is the prodrug moiety of formula (1A), (1B) or (2); X⁻ is a pharmaceutically acceptable anion, and any stereoisomer, salt, hydrate or solvate thereof. In case where both R² and R³ are a prodrug moiety of formula (1) or (2), pharmaceutically acceptable anion X⁻ can be double (e.g., 2 X⁻).

The prodrug may be, and any of the methods described herein may use, a prodrug of the formula (IX):

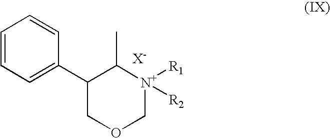

where R¹ is a hydrogen or a substituted or unsubstituted alkyl or prodrug moiety of formula (1A), (1B) or (2); R² is the prodrug moiety of formula (1A), (1B) or (2); X⁻ is a pharmaceutically acceptable anion, and any stereoisomer, salt, hydrate or solvate thereof. In case where both R¹ and R² are a prodrug moiety of formula (1A), (1B) or (2), pharmaceutically acceptable anion X⁻ can be double (e.g., 2 X⁻).

This invention also embraces all salts, polymorphs, crystals or non-crystalline forms of any of the prodrugs disclosed herein and methods of using the same and pharmaceutical compositions of any of the forgoing, such as when the compound is formulated with a pharmaceutically acceptable carrier. For all compounds, including prodrugs, disclosed herein, all stereoisomers are encompassed, including any diasteriomers, enantiomers or mixtures, such as racemic mixtures or mixtures containing an enanteomeric excess of one isomer. Those stereoisomers that retain appropriate biological function are particularly preferred, and may be present in pure or in substantially pure form.

Any of the prodrugs may be in a composition such as a pharmaceutical composition in substantially pure form. As used herein, “substantially pure” refers to material which is at least 50% pure (i.e., free from contaminants), more preferably at least 90% pure, more preferably at least 95% pure, more preferably at least 98% pure, more preferably at least 99% pure.

Any of the prodrugs may be present in a pharmaceutically acceptable salt which salts may be derived from a variety of organic and inorganic counter ions well known in the art and include, by way of example only, salts of organic or inorganic acids, such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, trifluoroacetic, and isethionic. The counterion (X⁻) in formulae (I)-(V) may be, but is not limited to, the particular counterions mentioned explicitly herein. Pharmaceutically acceptable salts can be made from the parent compound or prodrug, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 20th ed., Lippincott Williams & Wilkins, Baltimore, Md., 2000.

In one variation, the prodrugs exhibit enhanced solubility when compared to their parent drugs. For instance, the solubility of levorphanol free base was less than 1 mg/mL of pH 8 PBS. In contrast, the solubility of N-Phosphonooxymethyl levorphanol was approximately 137 mg/mL of pH 8 PBS.

In one variation, the prodrugs exhibit reduced or no bioactivity or reduced or no affinity for a receptor wherever applicable, when compared to their parent drugs. For instance, the IC₅₀ of N-Phosphonooxymethyl levorphanol and levorphanol tartrate at human mu receptor were 1.4E-08 M and 5.2E-10 M, respectively. In the same assay, inhibition constant, Ki, was 5.7E-09 and 2.2E-10 for N-phosphonooxymethyl levorphanol and levorphanol, respectively.

### Methods

Any of the methods described herein can employ one or more of the prodrugs described herein, including without limitation the prodrug N-phosphonooxymethyl levorphanol.

Methods for treating pain, psychological disorder, compulsive overeating or any pathological condition responsive to opioid, benzodiazepines, CNS stimulants, or anorexiants are described. In one variation, the method comprises the step of administering a prodrug in any dosage form such as tablet, capsule, oral solution, suspension, emulsion and the like for the treatment of pain. In some variations, the pain is selected from the group consisting of pain associated with surgery, trauma, postherpetic neuralgia, diabetic neuropathy, HIV-associated neuropathy, complex regional pain syndrome, osteoarthritis, rheumatoid arthritis, fibromyalgia and lower back pain. In other variations, the pain is selected from the group consisting of pain associated with nerve injury, stroke, multiple sclerosis, syringomyelia, epilepsy, spinal cord injury and cancer. In other variations, the psychological disorder is selected from the group consisting of transient or short term insomnia, acute stress reactions, episodic anxiety, generalized anxiety, adjustment disorder, severe panic disorder, agoraphobia, epilepsy, some motor disorders, acute psychoses, muscle spasms and seizures, dizziness, malaise, headache, pallor, ADHD. In yet other variations, the medical condition to be treated is compulsive overeating.

Also described is a method of delaying the onset of parent drug action in an individual in need of parent drug therapy, the method comprising administering to the individual an effective amount of an prodrug wherein the prodrug provides a slower onset of parent drug action as compared to the parent drug. In one variation, administration of the prodrug delays the onset of opioid activity by about 5 minutes or about 15 minutes or about 30 minutes or about 1 hour or about 2 hours or about 3 hours or about 4 hours or about 6 hours or about 8 hours or about 10 hours or about 12 hours or about 18 hours or more as compared to administration of the parent opioid.

Also described is a method of prolonging opioid or other parent drug activity in an individual in need of opioid or other parent drug therapy, the method comprising administering to an individual an effective amount of a suitable prodrug of the invention where the prodrug provides prolonged opioid activity as compared to the parent opioid or other parent drug. In one variation, administration of the opioid prodrug prolongs opioid activity by about 5 minutes or about 15 minutes or about 30 minutes or about 1 hour or about 2 hours or about 3 hours or about 4 hours or about 6 hours or about 8 hours or about 10 hours or about 12 hours or about 18 hours or more as compared to administration of the parent opioid.

Also described is a method of decreasing the abuse potential of an opioid in an individual in need of opioid therapy, the method comprising administering to an individual an effective amount of an opioid prodrug wherein the opioid prodrug is less susceptible to abuse when compared to administration of the parent opioid itself.

In a particular variation, the methods described employ the prodrug N-phosphonooxymethyl levorphanol.

### Methods of Making the Compounds

The prodrugs described herein may be made by any method, including linear synthesis or conjugation of a parent opioid to a prodrug moiety. Additional synthetic details may be found in the accompanying Examples section. A method of synthesis is also described in U.S. Pat. No. 5,985,856.

One method for the synthesis of the prodrugs involves a derivatizing reagent of the general form represented in Formula A.

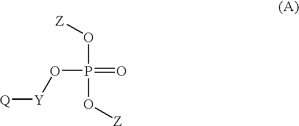

where Q in formula (A) represents any leaving group, Y is —CH₂—, —CH₂CH₂—, or —CH(CH₃)— and Z is selected so as to provide at least one phosphate protecting group. General Reaction Scheme (I) is given below:

### General Synthesis Scheme (I):

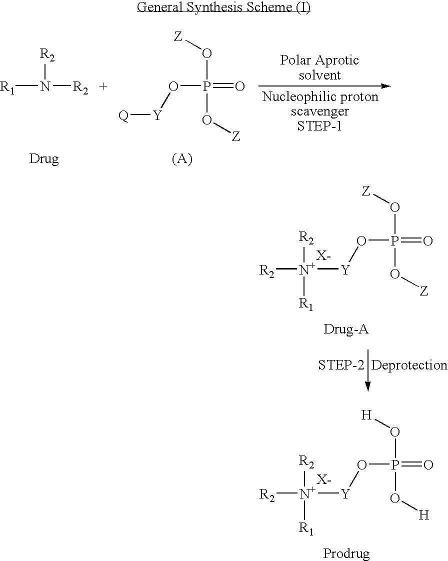

The reagent (A) may be mono-protected contrary to di-protected shown in the General Reaction Schemes (I). Likewise resulting intermediate or compound, Drug-A, may be mono-protected even when started with di-protected reagent (A). When mono-protected, one Z may be H. A phosphate protecting group(s), Z, is a group that blocks the reactive phosphate hydroxyl group(s) during coupling of reagent (A) with parent drug, thus allowing selective nucleophilic displacement of Q during Step-1 of General Synthesis Scheme (I). The phosphate protecting group can be selectively removed as depicted in Step-2 of the scheme (I). Examples of phosphate protecting groups include, but are not limited to, methyl, ethyl, isopropyl, tertiary butyl, benzyl, p-methoxybenzyl, allyl, 3′,5′-dimethoxybenzoin, p-hydroxyphenacyl, 2-cyanoethyl, 9-fluorenylmethyl, 2-(trimethylsilyl)ethyl, 2-(methylsulfonyl)ethyl, trityl and β-cyanoethyl etc. Further discussion of appropriate phosphate protecting groups may be found in Wuts, P. and Greene, J. Greene's Protective Groups in Organic Synthesis 4th ed. John Wiley & Sons, 2006; Greene, T. W., Wuts, G. M. P., Protective Groups in Organic Synthesis, 3rd Edition, New York, Wiley-Interscience, 1999 and McOmie, J. F. W., Protective Groups in Organic Chemistry. London and New York, Plenum Press, 1973.

A modified synthesis of Scheme I is also one aspect of this invention. Specifically, the following reaction conditions were determined to be an important aspect of the successful reaction: (1) crude di-tert-butyl chloromethyl phosphate was used in reactions; (2) repeated addition of large molar excess (4.8×) of di-tert-butyl chloromethyl phosphate every 2-days until reaction was completed; and (3) the de-protection step accomplished using approximately acetonitrile/1% TFA/water (2/1) as a suitable solvents system for decoupling which generally took 24 days to complete. Although it is primarily directed at modified synthesis according to Scheme I, the modification may also be applicable to certain aspects of a synthesis according to Scheme II below.

Alternatively to Scheme I, an amine moiety on parent drug may be combined with a ‘spacer’ moiety between parent drug and phosphoric acid moiety, which for example can be a —CH₂— containing two leaving groups. One leaving group allows attachment of “spacer” moiety with the parent drug as first step in synthesis of prodrug while second leaving group permits attaching phosphoric moiety to the intermediate resulted from first step of synthesis. Reaction Scheme II depicts this synthesis route.

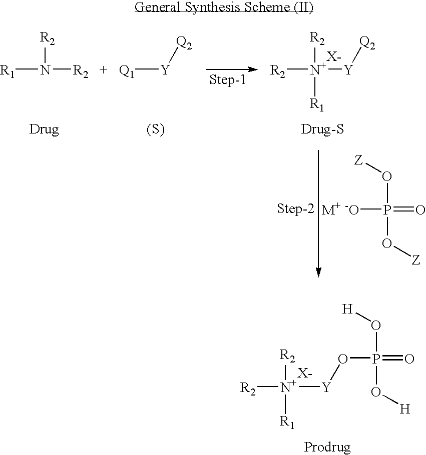

### Modes of Administration

For use herein, unless clearly indicated otherwise, the prodrugs may be administered to the individual by any available dosage form, route of administration, treatment regimen or formulation.

The prodrugs may be formulated in any dosage form or amount. On a molar basis, dosage amounts for prodrugs will be approximately in the same range as those for the parent drug; individual dose levels may range from micrograms to grams, depending on the parent drug and the tolerance and sensitivity of the individual

Formulations as described may include one or more prodrug and may also include one or more other compounds or drugs that are expected to or do have a therapeutic effect. For example, a formulation may comprise a prodrug of levophanol and acetaminophen, or a combination of hydrocodone and acetaminophen, or a combination of hydrocodone and ibuprofen or hydrocodone and aspirin or propoxyphene and aspirin and caffeine. The formulations may also comprise two or more prodrugs, such as a formulation comprising prodrugs of morphine and levorphanol or formulations comprising prodrugs of an opioid agonist and an opioid antagonist.

The prodrugs described here can be administered by oral, parenteral (intra-muscular, intraperitoneal, intravenous (IV) or subcutaneous injection), topical, transdermal (either passively or using iontophoresis or sonophoresis or electroporation or microneedles), transmucosal (e.g., nasal, vaginal, rectal, or sublingual) or pulmonary (e.g., via inhalation) routes of administration or using bioerodible inserts and can be formulated in dosage forms appropriate for each route of administration. Oral formulations can be immediate-release or delayed-release, site-specific, and may even contain any other drug or another APD e.g., opioid antagonist (in the case of an opioid agonist derivative), which is released in case the dosage form is subjected to any manner of use outside the prescribed instructions.

Topical

The prodrugs described above may be used for topical administration for cutaneous or transdermal delivery. A cutaneous topical dosage form is valuable in that alkaline phosphatase is expressed in the skin, and thus with continuous exposure to site of burns or trauma, the enzyme will slowly release amounts of opioid analgesic sufficient to induce local analgesia, but without significant systemic exposure. Moreover, the intrinsic reduction of attractiveness to abusers provided by the APD prodrugs will enable such topical formulations to be widely prescribed without undue fear of promoting opioid abuse. Thus, creams, gels or ointments may be prepared with pharmaceutical excipients including thickening agents and penetration enhancers intended for topical administration. Compositions may range from 0.01 to 20% by weight of the prodrugs. Exemplary penetration enhancers are: d-pipertone and oleic acid; 1-menthone and oleic acid; 1-menthone and ethyl oleate; 1-menthone and benzyl alcohol; ethylene glycol and 1-menthone; benzyl alcohol and oleyl alcoholic; 1-menthone and cetyl alcohol; 1,3-butanediol and oleic acid; diethylene glycol monoethyl ether and 1-menthone; ethelyne glycol and oleic acid; isopropyl myristate; oleyl alcohol and 1-3, butandiol; 1-menthone and isopropyl butyrate; 1-menthone and 1,3-butanediol; n-hexane and oleic acid; menthone and methanol; methylnonenoic acid and n-hexane; oleyl alcohol and propylene glycol; methylnonenoic alcohol and dimethylacetamide, stearyl alcohol, oleyl alcohol, linoleyl alcohol, linolenyl alcohol, caprylic alcohol, decal alcohol, laurel alcohol, Propylene glycol, polyethylene glycol, ethylene glycol, diethylene glycol, triathlon glycol, ethoxy digkycol, dipropylene glycol, glycerol, propanediol, butanediol, pentanediol, hexanetriol 2-lauryl alcohol, myristyl alcohol, cetyl alcohol, capric acid, lauric acid, myristic acid, stearic acid, oleic acid, caprylic acid, valeric acid, heptanoic acid, pelagonic acid, caproic acid, isovaleric acid, neopentanoic acid, trimethyl hexanoic acid, neodecanoic acid, isostearic acid, neoheptanoic acid, neononanoic acid, isopropyl n-decanoate, isopropyl palmitate, octyldodecyl myristate, ethyl acetate, butyl acetate, methyl acetate, isopropyl n-butyrate, ethylvalerate, methylpropionate, diethyl sebacate, ethyl oleate, isopropyl n-hexanoate, isopropyl myristate, urea, dimethylacetamide, diethyltoluamide, dimethylformamide, dimethyloctamide, dimethyldecamide, 1-hexyl-4-methoxycarbonyl-2-pyrrolidone, 1-lauryl-4-carboxy-2-pyrrolidone, 1-methyl-4-carboxy-2-pyrrolidone, 1-alkyl-4-imidazolin-2-one, 1-methyl-2-pyrrolidone, 2-pyrrolidone, 1-lauryl-2-pyrrolidone, 1-hexyl-4-carboxy-2-pyrrolidone, 1-methyl-4-methoxycarbonyl-2-pyrrolidone, 1-lauryl-4-methoxycarbonyl-2-pyrrolidone, dimethylsulfoxide, decylmethylsulfoxide, N-cocoalkypyrrolidone, N-dimethylaminopropylpyrrolidone, N-tallowalkylpyrrolidone, N-cyclohexylpyrrolidone, 1-farnesylazacycloheptan-2-one, 1-geranylgeranylazacycloheptan-2-one, fatty acid esters of -(2-hydroxyethyl)-2-pyrrolidone, 1-geranylazacycloheptan-2-one, 1-dodecylazacycloheptane-2-one (Azone®), 1-(3,7-dimethyloctyl)azacycloheptan-2-one, 1-geranylazacyclohexane-2-one, 1-(3,7,11-trimethyldodecyl)azacyclohaptan-2-one, 1-geranylazacyclopentan-2,5-dione, 1-farnesylazacyclopentan-2-one, benzyl alcohol, butanol, pentanol, hexanol, octanol, nonanol, decanol, ethanol, 2-butanol, 2-pentanol, propanol, diethanolamine, triethanolamine; hexamethylenelauramide and its derivatives, benzalkonium chloride, sodium laurate, sodium lauryl sulfate; cetylpyridinium chloride, citric acid, succinic acid, salicylic acid. sylicylate Cetyltrimethyl ammonium bromide, tetradecyltrimethylammonium bromide; octadecyltrimethylammonium chloride; dodecyltrimethylammonium chloride, hexadecyltrimethylammonium chloride, Span 20, Span 40, Span 60, Span 80, Span 85, Poloxamer231, Poloxamerl82, Poloxamerl84), Brij 30, Brij 35, Brij 93, Brij 96, Span 99, Myrj45, Myrj51, Myrj52, Miglyol 840, glycholic, sodium salts of taurocholic, lecithin, sodium cholate, desoxycholic acids, D-limonene, α-pinene, β-carene, α-terpineol, terpinen-4-ol, carvol, carvone, pulegone, piperitone, Ylang ylang, menthone, anise, chenopodium, eucalyptus, limonene oxide, a-pinene oxide, cyclopentene oxide, 1,8-cineole, cyclohexene oxide, N-heptane, N-octane, N-nonane, N-decane, N-undecane, N-dodecane, N-tridecane, N-tetradecane, N-hexadecane and essential oils (e.g., tea tree oils). The prodrugs described above may be formulated in patches. Patch designs may include drug in adhesive matrix, micro liquid reservoir or multilayered liquid reservoir. Other compositions may include nano- or microparticulate suspensions in an adhesive matrix. The liquid reservoir patches may be designed such that the prodrug is reconstituted at the time of application. The reconstitution will simply involve breaking the barrier between drug substance and liquid reservoir and gently shaking the patch if warranted.

Ointments typically contain a conventional ointment base selected from the four recognized classes: oleaginous bases; emulsifiable bases; emulsion bases; and water-soluble bases. Lotions are preparations to be applied to the skin or mucosal surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Creams, as known in the art, are viscous liquid or semisolid emulsions, either oil-in-water or water-in-oil. Topical formulations may also be in the form of a gel, i.e., a semisolid, suspension-type system, or in the form of a solution.

Oral

The prodrugs may be delivered orally. For example, formulations may include enteric coatings or properties which make it difficult to extract the prodrug from the oral formulation (which confer an additional abuse deterrence besides that intrinsic to the molecule).

Solid dosage forms for oral administration include capsules, tablets, pills, powders, and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating, agents such as magnesium stearate. In the case of capsules, tablets, and pills, the dosage forms may also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

Mucosal

Similarly to oral bioavailability benefits, mucosal delivery methods may demonstrate improved performance for administrations such as bladder instillation or oral mucositis. These formulations may include creams, gels, ointments or oil/water emulsions. Compositions for rectal or vaginal administration are preferably suppositories which may contain, in addition to the active substance, excipients such as cocoa butter or a suppository wax. Compositions for nasal or sublingual administration are also prepared with standard excipients well known in the art. Buccal delivery is also embraced, and compositions for buccal administration may also be prepared as known in the art (e.g., formulated as capsules, gums or lozenges).

Parenteral

Some parent drugs, e.g., opioid compounds, are moderately soluble in aqueous solutions. The APD prodrugs compounds described here may display improved water solubility, and thus can be administered in reduced dose volumes. For injectable or parenteral formulations, this will alleviate concerns that crystals will form at the site of injection or administration. Parent drug derivatives may be formulated in suitable aqueous suspensions or solutions intended for injection. Formulations may include suitable buffers and stabilizing excipients together with water, saline, or other sterile injectable medium for injection.

Preparations according to this invention for parenteral administration include sterile aqueous and non-aqueous solutions, suspensions, or emulsions. Examples of non-aqueous solvents or vehicles are propylene glycol polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions.

Inhalation

As alkaline phosphatase is produced by cells present in the lungs, wherever appropriate the prodrugs described here could be metabolically activated following delivery by inhalation. For example, several investigational inhalation opioid formulations are undergoing clinical evaluation, but the prodrug compounds described here may possess advantages with respect to these investigation formulations of existing compounds. Advantages could follow from the enhanced water solubility, potential reductions in local irritancy or toxicity, or in the controlled., gradual delivery expected for molecules, which require metabolic activation.

### Kits

Kits are also encompassed and may employ any of the compounds or prodrugs disclosed herein and instructions for use. Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may optionally include a set of instructions, generally written instructions, although electronic storage media (e.g., magnetic diskette or optical disk) containing instructions are also acceptable, relating to the use of component(s) of the methods of the present invention

The following examples are provided to illustrate various embodiments of the invention and are not intended to be limiting of the scope of the appended claims or to otherwise limit the invention in any manner.

All references listed herein, including any patent, patent publication, patent application, journal article or text are incorporated herein by reference in their entirely the same as if each and every reference were separately and individually incorporated herein by reference in its entirety.

### EXAMPLES

### Example 1

### Synthesis of N-Phosphonooxymethyl Levorphanol

Preparation of di-tert-butyl chloromethyl phosphate (3). Di-tert-butyl chloromethyl phosphate was prepared according to reaction Example Scheme 1.

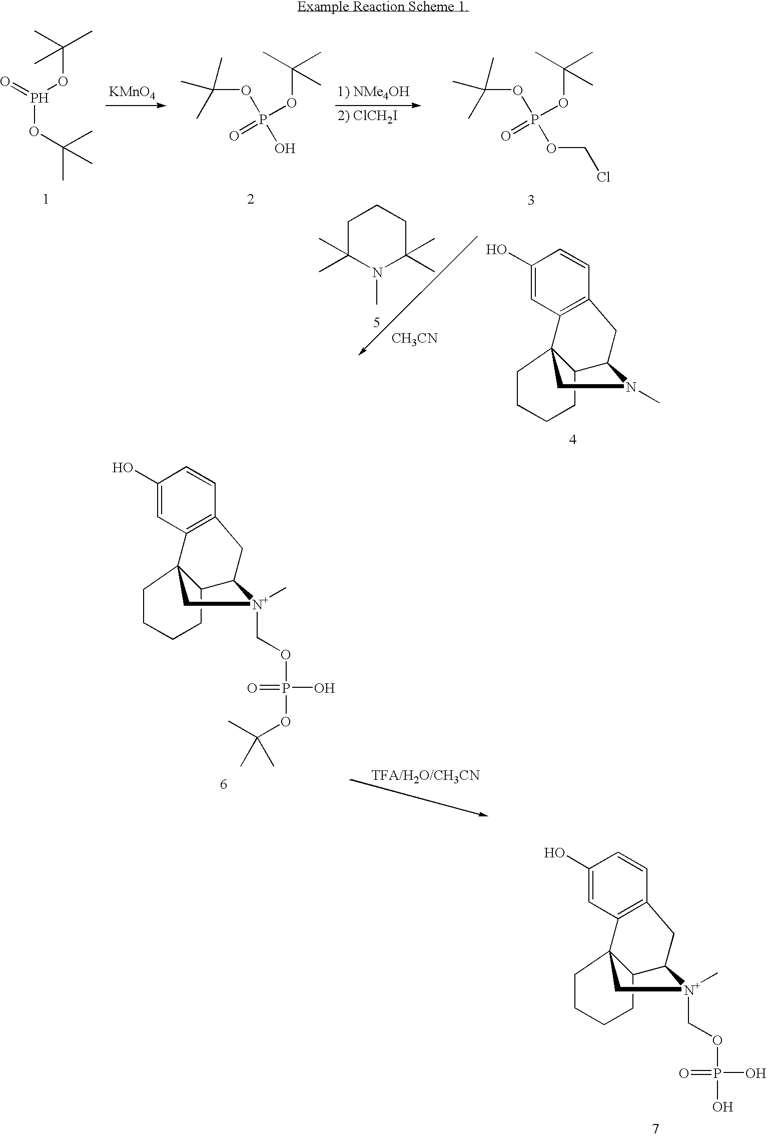

Di-tert-butyl phosphate (2). To a stirred solution, in an ice bath, of di-tert-Butyl phosphite (1) (25 g, 128 mmol) and potassium bicarbonate (7. g, 77.9 mmol) in 28 mL water was added over one hour about 6 equal portions of powdered potassium permanganate (34.7 g, 220 mmol). The purple mixture was stirred an additional 45 minutes at room temperature. Norite (5 g) was added, and the resulting mixture was stirred at 60° C. The mixture was filtered through a Celite cake and the cake was washed with 3×50 mL water. The combined filtrate was mixed with 10 g of Norite, and stirred for 30 minutes at 60° C. The mixture was again filtered through Celite and the filter cake was washed with 50 mL of warm water. The clear filtrate was chilled to about 0° C. on an ice water/acetone bath and slowly acidified with 55 mL of concentrated hydrochloric acid with stirring (a white precipitate formed). The di-tert-butyl phosphate (2) was filtered and washed with 50 mL ice water. The solid was vacuum dried overnight to give 23 g of di-tert-butyl phosphate (2) (85%). MS 209 (M−1). The solid was stored at −10° C. under argon.

Di-tert-butyl chloromethyl phosphate (3). In 50 mL of acetone was stirred (cloudy solution) 2.68 g (12.7 mmol) of di-tert-butyl phosphate (2) on an ice bath. To the mixture was added 2.30 g (12.7 mmol) of tetramethylammonium hydroxide pentahydrate in 10 mL water with stirring to give a clear solution. The solution was evaporated to a cloudy thick oil and dissolved in 50 mL of dimethoxyethane. The cloudy dimethoxyethane was evaporated to cloudy semi-solid and placed under high vacuum until the residue formed a solid. The solid was slurried in 50 mL of refluxing dimethoxyethane and 2 g (141.7 mmol) of chloroiodomethane was added. An almost clear light yellow solution formed and refluxing was continued for 1 hour. A precipitate formed in about 5 minutes, which was yellow at first and then white in color. The hot mixture was filtered and the dimethoxyethane evaporated to give 1.90 g (58%) of a light yellow oil (3). NMR: ¹H (300 MHz, CDCl₃) δ 1.57 (s, 9H), 5.63 (d, 2H, J=15 Hz); ³¹P NMR (300 MHz, CDCl₃) δ−10.8 (s). The NMR showed about 10-15% bis-di-tert-butyl methyl phosphate impurity that could not be removed by flash column chromatography (silica gel, ethyl acetate/hexane eluent), even with 100% ethyl acetate eluent. ¹H (300 MHz, CDCl₃) δ 1.5 (s, 18H), 1.50 (s, 18H), 5.44 (t, 2H, J=12 Hz); ³¹P NMR (300 MHz, CDCl₃) δ −11.1 (s).

### Preparation of N-Phosphonooxymethyl Levorphanol (7)

Levorphanol (4). To 900 g (203 mmol) of levorphanol tartrate dihydrate salt as added 20 mL of 10% sodium bicarbonate and the solution was extracted with 3×30 mL of chloroform. The chloroform solution was dried over magnesium sulfate, filtered and evaporated to dryness to yield 495 mg (94%) of white solid (4). NMR: ¹H (300 MHz, CDCl₃) δ 6.86 (d, 1H, J=8.1 Hz), 6.65 (d, 1H, J=2.7 Hz), 6.54 (dd, 1H, J=8.1, 2.7 Hz), 2.87 (d, 1H, J=18.6), 2.73 (m, 1H), 2.54 (dd, 1H, J=18.6, 6.0 Hz), 2.37 (ddd, 1H), 3.00 (s, 3H), 2.22 (m, 1H), 2.06 (dt, 1H, J=12.3, 3.3 Hz), 1.74 (dt, 1H, J=12.3, 3.3 Hz), 1.64 (dt, 1H, J=12.6, 4.8 Hz), 1.55 (m, 1H), 1.2-1.46 (m, 6H).

Mono-tert-butyl N-(phsphonooxymethyl)levorphanl hydrochloride (6). A sample of 450 mg (1.75 mmol) of levorphanol (4) was added to a solution of 540 mg (2.10 mmol) of di-tert-butyl chloromethylphosphate (3) and 326 mg (2.10 mmol) of 1,2,2,6,6-pentamethylpipperidine (5) in 20 mL anhydrous acetonitrile (dried over 3 Å molecular sieves), flushed with argon and sealed with a stopper. The solution was stirred in a 43° C. oil bath for 2 days and a second addition of 540 mg (2.10 mmol) di-tert-butylchloromethylphosphate (3) and 326 mg (2.10 mmol) of 1,2,2,6,6-pentamethylpipperidine (5) was added. The flask was flushed with argon and the sealed flask was stirred for another 2 days in a 43° C. oil bath at which time a third addition of 540 mg (2.10 mmol) di-tert-butyl chloromethylphosphate (3) and 326 mg (2.10 mmol) of 1,2,2,6,6-pentamethylpipperidine (5) was added and the sealed flask under argon was stirred in an oil bath at 43° C. for 2 more days. The reaction was evaporated to an oil and 20 mL ethyl ether was added with stirring. The white solid was filtered and vacuo dried (hygroscopic) to yield 800 mg (94%) of crude product (6). HPLC: Supelco Discovery RP Amide 250×4 mm C16 5μ column; flow 1 mL/min; detection UV MaxPlot 220-400 nm; solvent: 65% 0.0025 N ammonium formate pH=6.5/35% acetonitrile: retention time 3.59; 46% unknown (no uv of levorphanol derivative), retention time 3.96; 7% levorphanol-phosphate, retention time 6.23; 37.3% mono-tert-butyl-levorphanol-phosphate; retention time 18.8; 8.85% levorphanol.

Preparative separation of mono-tert-butyl-levorphanol-phosphate (6). The crude mono-tert-butyl-levorphanol phosphate (6) was purified by injecting 260 mg in seven injections (5 to 75 mg) on a Waters' Sunfire C₁₈ OBD prep column (10 ml 10×150 mm). The product was eluted with a gradient of 70% 0.03 N (pH=5.4) ammonium formate/30% acetonitrile for 35 minutes, then with a 60/40 mixture (60% 0.03 N (pH=5.4) ammonium formate/40% acetonitrile) over 3 minutes and with the 60/40 mixture for 17 minutes at 5 mL/min. The product eluted at about 15 to 20 minutes and was manually collected. The seven fractions were combined and evaporated to dryness at 30 to 35° C. under high vacuum. The solid was under vacuo at 30-35° C. overnight to remove the ammonium formate to give 120 mg of crude product. HPLC showed 72% mono-tert-butyl-levorphanol-phosphate (6) and 28% levorphanol (4). NMR showed mono-tert-butyl-levorphanol-phosphate (6) and about one equivalent of ammonium formate. HPLC of fractions before evaporation showed 96% product (6) and 2.6% levorphanol (4). NMR: ¹H (300 MHz, D₂O) δ 8.309s, 1H), 7.07 (d, 1H, J=8.4 Hz), 6.81 (d, 1H, J=2.4 Hz), 6.71 (dd, 1H, J=8.4, 2.4 Hz), 5.71 (t, 1H, J=9 Hz), 4.97 (t, 1H, J=8.4 Hz), 3.72 (s, 1H), 3.22 (m, 2H), 3.03 (s, 3H), 2.84 (dt, 1H, J=18.3, 3.3 Hz), 2.77 (s, 1H), 2.33 (d, 1H, J=12 Hz), 2.22 (d, 1H, J=12 Hz), 1.94 (dt, 1H, J=15, 3.9 Hz), 1.2-1.62 (m, 6H), 1.35 (s, 9H), 0.90*1.17 (m, 2H). MS: ESI positive ion m/z 424 (M⁺).

N-Phosphonooxymethyl Levorphanol (7). The mono-tert-butyl-levorphanolphosphate (6) was placed in 10 mL acetonitrile/1% TFA/water (2/1) and was stirred at room temperature for 24 days until HPLC showed the tert-butyl group had been removed. The solution was evaporated to dryness. The N-phosphonooxymethyl levorphanol (7) was purified on a Waters' Sunfire Cl₈ OBD preparative column (10 mμ. 10×150 mm) eluding with 0.0025 ammonium formate (pH=6.5/acetonitrile. A gradient of 70/30 to 60/40 was used (70% 0.03 N (pH=5.4) ammonium formate/30% acetonitrile). With four injections, the largest peak was collected and evaporated to 70 mg of a glass at room temperature. The glass was titrated with acetonitrile to give a white solid. The solid was filtered and vacuo dried (very hygroscopic) to yield 40 mg of desired product (7). NMR: ¹H (300 MHz, D₂O) δ 8.34 (s, 1H), 7.03 (d, 1H, J=8.4 Hz), 6.76 (d, 1H, J=2.4 Hz), 6.69 (dd, 1H, J=8.4, 2.4 Hz), 5.04 (t, 1H, J=7.5 Hz), 4.95 (t, 1H, J=7.5 Hz), 3.67 (s, 1H), 3.18 (m. 3H), 2.98 (s, 3H), 2.72 (dt, 1H, J=13.8, 3.9 Hz), 2.27 (m, 2H), 1.95 (dt, 1H, J=15, 4.5 Hz), 1.2-1.60 (m, 6H), 0.82-1.12 (m, 2H). MS: ESI positive ion m/z 368 (M⁺). HPLC: Supelco Discover RP Amide 250×4.6 mm C₁₆ 5μ column; flow 1 mL/min; detection UV MaxPlot 220-400 nm; solvent; 85% 0.0025 N ammonium formate pH=6.5/15% acetonitrile: retention time 4.53; 98.6% product (7). HPLC: Sunfire 250×4.6 mm C₁₈ 5μ column; flow 1 mL/min; detection UV 82 nm; solvent; 85% 25 mM ammonium formate pH=4/26% acetonitrile: retention time 9.37; 88.9% product (7).

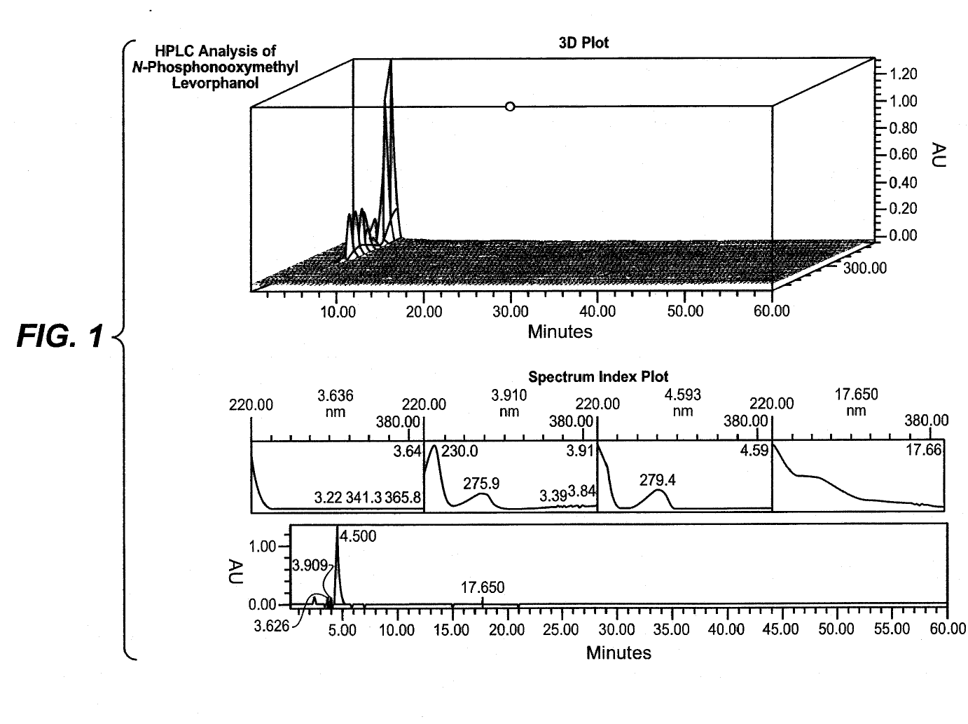

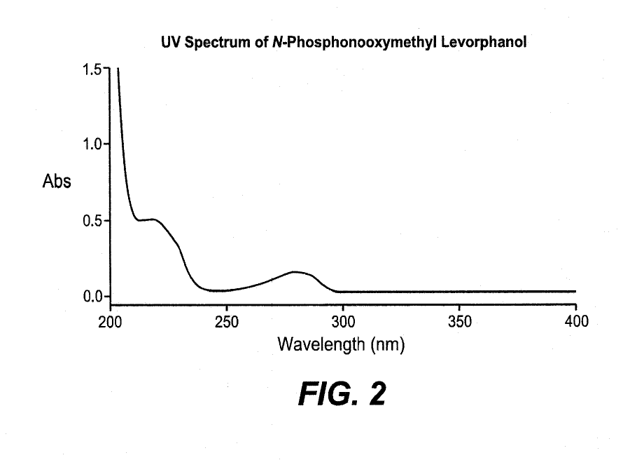

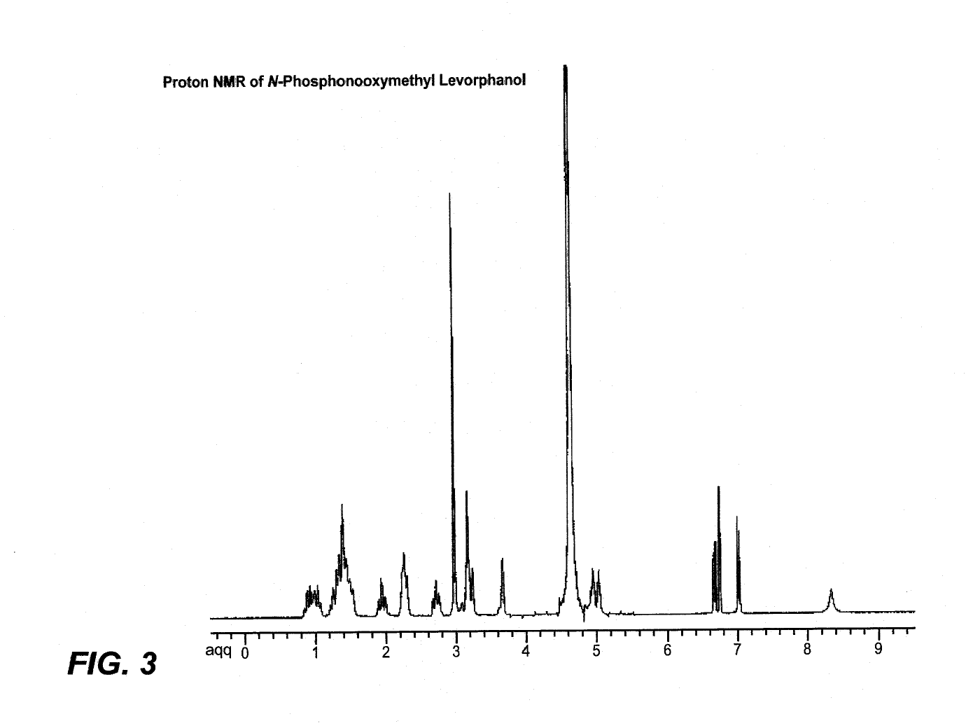

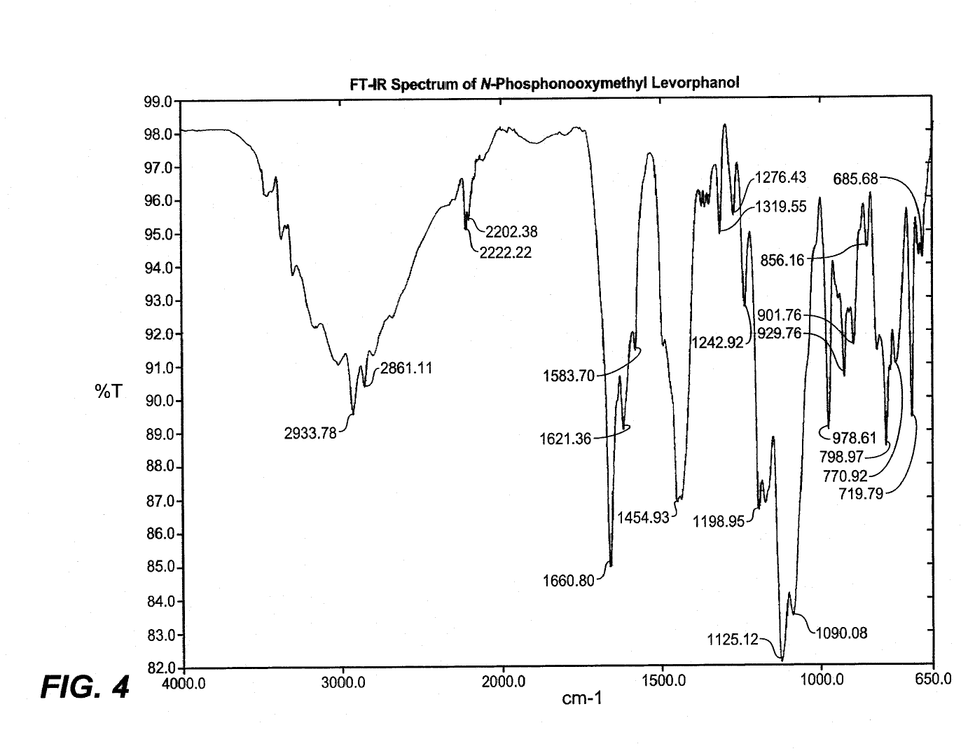

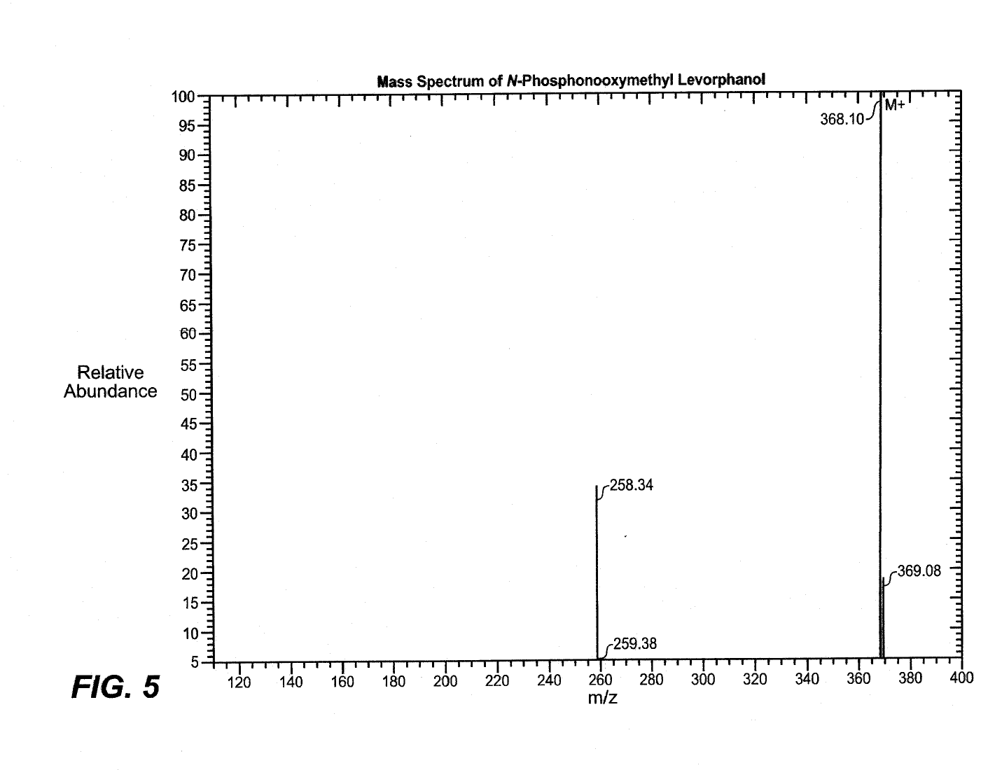

See FIGS. 1-5 for HPLC, UV spectrum, ¹H NMR, FT-IR and mass spectroscopy data for N-phosphonooxymethyl levorphanol.

### Example 2

### Chemical Hydrolysis Studies

Aqueous stability of N-phosphonooxymethyl levorphanol at pHs 1.2, 6, and 8 at 37° C. by was determined by comparing the sample solutions at specific time points as shown in data tables below. PBS solutions at various pHs (1.2, 6 and 8) were prepared by adjusting 60-mL volumes of PBS maintained at 37° C. with a dropwise addition of concentrated phosphoric acid and/or 0.01 N NaOH, and monitoring with a pH meter calibrated between pH 4 and 7. Solubility of N-phosphonooxymethyl levorphanol and Levorphanol (free base) in PBS at pH 8 and was found to be approximately 137 mg/mL. Levorphanol (free base) was soluble by sonication at less than 1 mg/mL at 37° C. A 50 μL sample was taken for the t=0 time point. The remaining solution was then divided into five aliquots (175 μL each) in microcentrifuge vials labeled according to sampling time-points. Air-tight sealed vials were then incubated at 37° C. in a temperature-controlled water bath. At each time-point, the appropriate vial was removed from the water bath, and a 50 μL sample drawn for HPLC analysis. HPLC analysis data tables showing assay conditions, sampling time points, and percent of N-phosphonooxymethyl levorphanol remaining in solution at that time point are given in Table 1 below:

Tables 1-3: Chemical Hydrolysis Studies of N-phosphonooxymethyl levorphanol

TABLE 1
Stability of N-phosphonooxymethyl levorphanol at
pH 1.2 PBS at 37° C.
(Concentration 1 mg/mL of PBS)

| | | Percentage of N- |
|---|---|---|
| Total Time | | phosphonooxymethyl |
| (Hours) | Peak Area | levorphanol remaining |
| 0 | 3290963 | 100.0 |
| 0.5 | 3668745 | 111.5 |
| 1 | 3440663 | 104.4 |
| 3 | 3648129 | 110.9 |
| 5 | 3659530 | 111.1 |
| 23 | 3648090 | 110.9 |

TABLE 2
Stability of N-phosphonooxymethyl levorphanol at pH 6 PBS at 37° C.
(Concentration 1 mg/mL of PBS)

| | | Percentage of N- |
|---|---|---|
| Total Time | | phosphonooxymethyl |
| (Hours) | Peak Area | levorphanol remaining |
| 0 | 3397388 | 100.0 |
| 0.5 | 3445461 | 101.4 |
| 1 | 3476596 | 102.3 |
| 3 | 3544141 | 104.3 |
| 5 | 3545017 | 104.3 |
| 22 | 3669831 | 108.0 |

TABLE 3
Stability of N-phosphonooxymethyl levorphanol at pH 8 PBS at 37° C.
(Concentration 1 mg/mL of PBS)

| | | Percentage of N- |
|---|---|---|
| Total Time | | phosphonooxymethyl |
| (Hours) | Peak Area | levorphanol remaining |
| 0 | 3497899 | 100.0 |
| 0.5 | 3519390 | 100.6 |
| 1 | 3506960 | 100.3 |
| 3.5 | 3492592 | 99.8 |
| 6 | 3515800 | 100.5 |
| 24 | 3625625 | 103.7 |

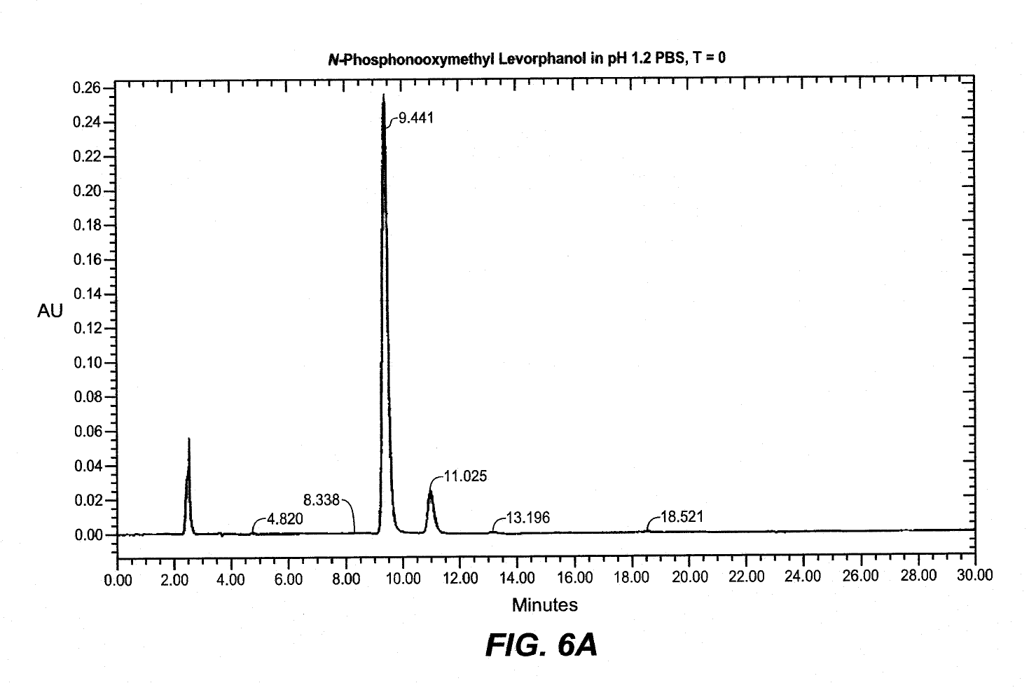

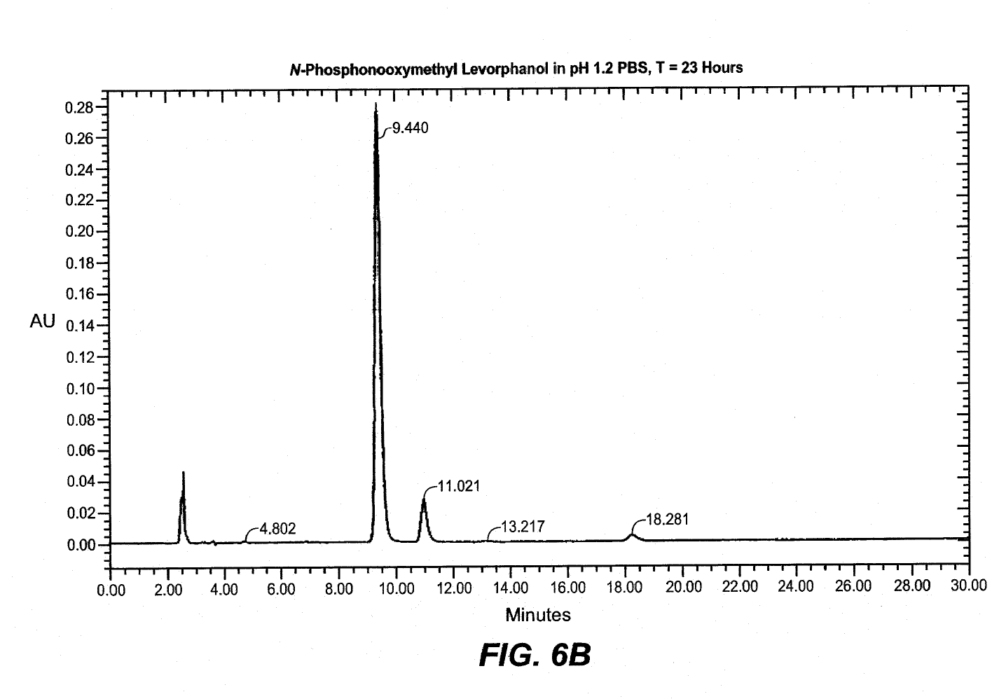

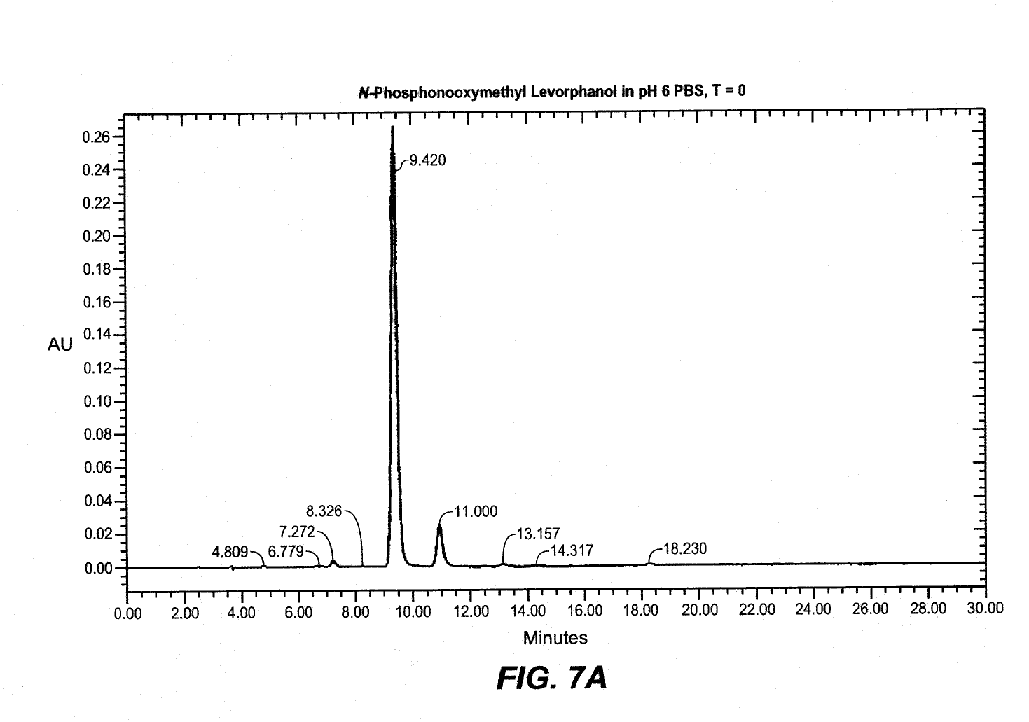

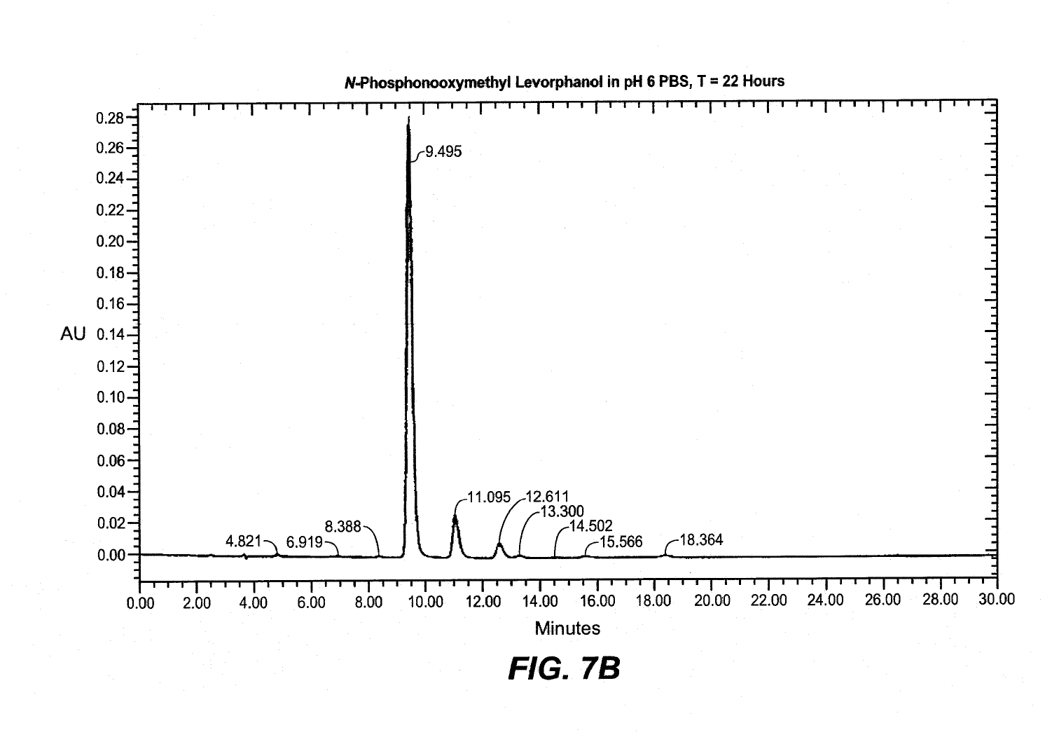

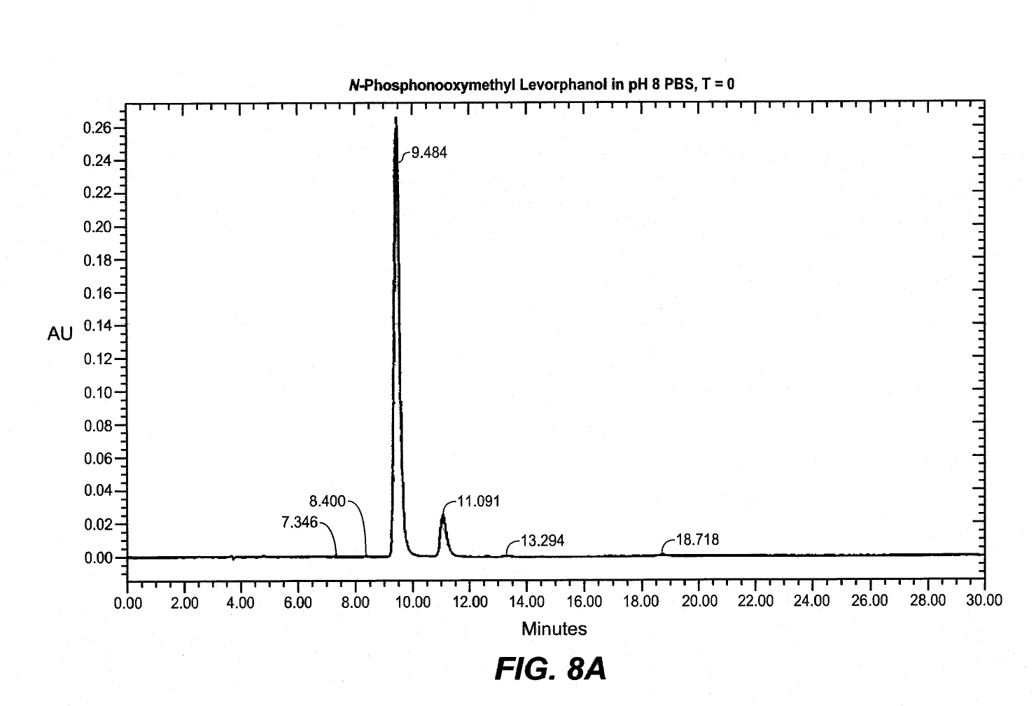

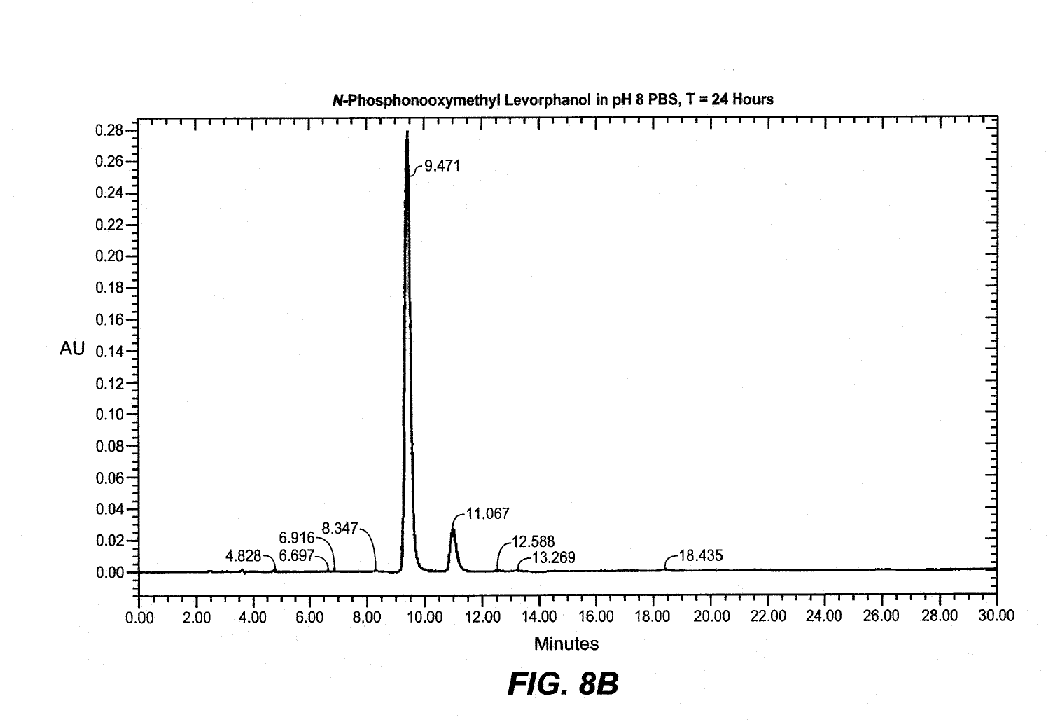

See FIG. 6 for chemical stability data of N-phosphonooxymethyl levorphanol at pH 12. See FIG. 7 for chemical stability data of N-phosphonooxymethyl levorphanol at pH 6. See FIG. 8 for chemical stability data of N-phosphonooxymethyl levorphanol at pH 8.

### Example 3

### Enzymatic Hydrolysis Studies

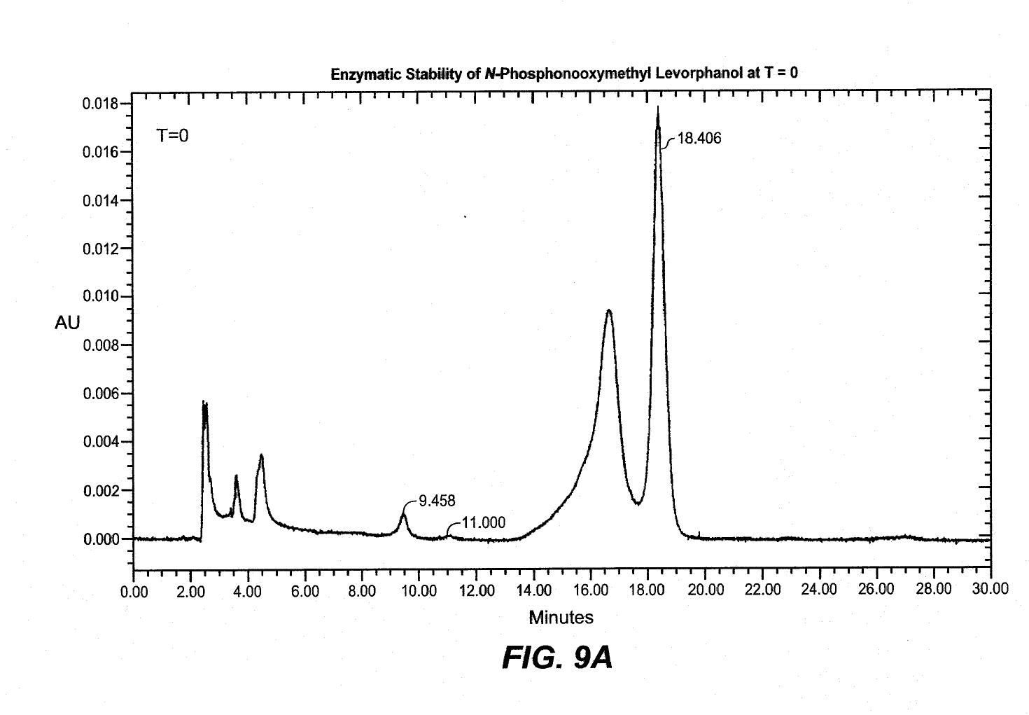

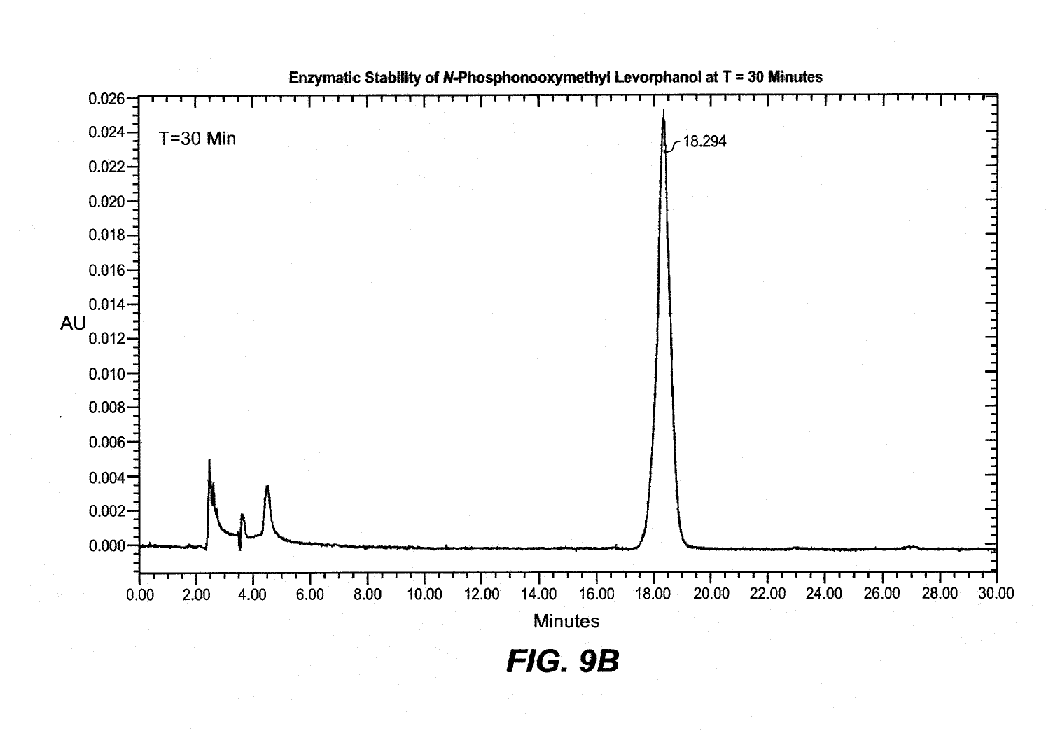

Stability of N-phosphonooxymethyl levorphanol in the presence of alkaline phosphatase (EC 3.1.3.1; Sigma-Aldrich Catalog #P7923) at pH 8 at 37° C. by HPLC was determined by comparing the area under the peak of sample solutions at specific time points (0, 0.5, 1.25, 3, 5.5 and 23 hours) with a freshly prepared sample peak area. Alkaline Phosphatase (400 units) was added to 10 mL of Alkaline Phosphatase Stabilizing Buffer (APSB; Sigma-Aldrich Catalog #A4955) maintained at 37° C. Solution pH was verified by dropping 25 μL of solution onto calibrated Color-pHast Indicator Strips (pH 2-9, Darmstadt, Germany) and checking the color against the calibration scale. N-phosphonooxymethyl levorphanol (3 mg) was added to 3 mL of the above enzyme+buffer solution and incubated at 37° C. At each sampling time point, a 200-μL aliquot was drawn into labeled micro centrifuge tubes, each containing 50 μL of Alkaline Phosphatase Stop Solution (APSS; Sigma-Aldrich Catalog #A5852). Acetonitrile (400 μL) was added to each micro centrifuge tube, vortexed briefly, and centrifuged at 12,000 rpm for 5 min. An aliquot (200 μL) of resulting supernatant was removed from each tube, being careful not to disturb the enzyme pellet at the bottom and transferred to HPLC vials containing low volume inserts. Reference N-phosphonooxymethyl levorphanol solutions in the range of 50 to 150% of the assay concentration—i.e. 1 mg/mL—were prepared and interspersed between the sample injections, and immediately prior to chromatography, by dissolving each reference in 1 mL of 60/40 acetonitrile/APSB solution, and chromatograph under the HPLC. When stored at 37° C. in the presence of alkaline phosphatase (AP) at a ratio of 40 units AP per mg, N-phosphonooxymethyl levorphanol appeared to hydrolyze to levorphanol within 30 minutes, as evidenced by the drop in peak area at 9.4 min (N-phosphonooxymethyl levorphanol peak) and appearance of broad peak at 18 min (levorphanol peak). FIG. 9 shows a chromatogram of an extract sampled at time zero (a, upper image) and after 30 min (b, lower image). The hydrolysis appeared to begin immediately. As shown in FIG. 1a, only 1.4% of the total peak area is N-phosphonooxymethyl levorphanol at time zero. There were no measurable amounts of N-phosphonooxymethyl levorphanol after 30 minutes. See FIG. 9 for enzymatic stability data of N-phosphonooxymethyl levorphanol.

### Example 4

### Synthesis of N-Phosphonooxymethyl Morphine

### Preparation of mono-tert-butyl-N-(phosphonooxymethyl)morphine

To 100 mg (0.35 mmol) of morphine was added a solution of 110 mg (0.426 mmol) di-tert-butyl chloromethylphosphate plus 75 mg (0.48 mmol) of 1,2,2,6,6-pentamethylpiperidine in 5 mL anhydrous acetonitrile (dried over 3 Å molecular sieves), flushed with argon and sealed. The solution was stirred in a 45° C. oil bath for 2 days followed by a 2^{nd} addition of 110 mg (0.426 mmol) of di-tert-butyl chloromethylphosphate plus 70 mg (0.45 mmol) of 1,2,2,6,6-pentamethylpiperidine, flask flushed with argon, sealed, stirred for 2 days in a 45° C. oil bath. It was then followed by a third addition of 110 mg (0.426 mmol) of di-tert-butyl chloromethylphosphate plus 70 mg (0.45 mmol) of 1,2,2,6,6-pentamethylpiperidine, and the flask sealed under argon, stirred in an oil bath at 45° C. for 2 more days. The reaction was evaporated to an oil and 20 mL of ethyl ether was added with stirring. The white solid was filtered and vacuo dried (hygroscopic) to yield 200 mg (110% yield). The HPLC and MS (ESI+) showed a mixture of mono-t-butyl (major), di-t-butyl, t-butyl-free compound, and morphine starting material.

Preparation of N-Phosphonooxymethyl Morphine: The crude mono-t butyl-morphine-methylphosphate in 10 mL of acetonitrile/1% TFA/H2O (1/3) was stirred at room temperature for 6 days until MS showed that most of t-butyl protecting group had been removed. The solution was evaporated to dryness. The title compound was purified on a Waters' Sunfire C₁₋₈ OBD prep column (10μ, 10×150 mm) eluting with 0.0025 ammonium formate (pH=6.5)/acetonitrile using a gradient of 80/20 to 60/40. With two injections, the largest peak was collected and evaporated to a glass at room temperature. The HPLC showed ˜25% impurity, and the residue was re-chromatographed with one ejection using the same system. The main peak was evaporated to dryness to give 20 mg yield after triturating with acetonitrile as pale white solid (13%).

NMR: ¹H (400 MHz) (D₂O) δ 8.27 (s, 1H), 6.61 (d, 1H, J=8.2 Hz), 6.52 (d, 1H, J=8.2 Hz), 5.56 (d, 1H, J=10.0 Hz), 5.26 (do. 1H, J=10.0, 2.0 Hz). 5.17 (t, 1H, J=7.0 Hz), 4.94 (t. 1H, J=6.9 Hz), 4.92 (dd, 1H, J=6.7, 1.0 Hz), 4.23 (m, 2H), 3.35 (m, 3H), 3.14 (dt, 1H, J=4.1, 17.8 Hz), 3.10 (s, 3H), 2.83 (dd, 1H, J=6.3, 20.3 Hz), 2.40 (dt, 1H, J=4.7, 14.7 Hz), 1.86 (dd, 1H, J=12.2, 2.7) pap.

NMR: ¹³C (100 MHz) (D₂O) δ 145.4, 138.3, 133.1, 129.4, 125.8, 123.0, 120.3, 117.8, 90.3, 81.6, 65.5, 64.3, 51.9, 49.3, 41.3, 33.2, 29.4 and 23.2, pap.

MS: (ESI positive ion) m/z 396 (M+)

### Example 5

### Synthesis of N-Phosphonooxymethyl Fentanyl

Preparation of Fentanyl free base: A sample of 100 mg (1.89 mmol) of fentanyl citrate salt was dissolved in 20 mL of 10% sodium bicarbonate and extracted with 3×30 mL of chloroform. The chloroform was dried over magnesium sulfate, filtered, and evaporated to dryness to yield 60 mg (94%) of white solid.

Preparation of Mono-tert-butyl-N-Phosphonooxymethyl fentanyl: To 60 mg (0.179 mmol) of fentanyl was added a solution of 65 mg (0.251 mmol) di-tert-butyl chloromethylphosphate plus 55 mg (0.354 Menlo) of 1,2,2,6,6-pentamethylpiperidine in 5 mL of anhydrous acetonitrile (dried over 3 Å molecular sieves), flushed with argon and sealed with a stopper. The solution was stirred in a 45° C. oil bath for 2 days, followed by a 2^{nd} addition of 65 mg (0.251 mmol) of di-tert-butyl chloromethylphosphate plus 55 mg (0.35 mmol) of 1.2,2,6,6-pentamethylpiperidine and flask flushed with argon, sealed and stirred for 2 days in a 45° C. oil bath. It was then followed by a third addition of 65 mg (0.25 mmol) of di-tert-butyl chloromethylphosphate plus 55 mg (0.35 mmol) of 1,2,2,6,6-pentamethylpiperidine, and flask flushed with argon, sealed and stirred in an oil bath at 45° C. for 4 more days. The reaction was evaporated to an oil and 20 mL of ethyl ether added with stirring. The white solid was filtered and vacuo dried (hygroscopic) to yield 75 mg (75%). The HPLC and MS (ESI+) showed a mixture of mono-t-butyl (major), di-t-butyl, t-butyl free compound, and fentanyl starting material.

Preparation of N-Phosphonooxymethyl fentanyl: the crude mono-t-butyl-fentanyl-methylphosphate in 10 mL acetonitrile/1% TFA/H₂O (1/3) was stirred at room temperature for 6 days until MS showed that most of t-butyl had been removed. The solution was evaporated. The phosphate was purified on a Waters' Sunfire C₁₋₈ OBD prep column (10μ, 10×150 mm) eluting with 0.0025 ammonium formate (pH=6.5)/acetonitrile using a gradient of 80/20 to 60/40. With one injection, the largest peak was collected and evaporated to a glass at room temperature. The HPLC showed 2% impurity. The residue after triturating with acetonitrile gave 5 mg (7%) of desired product.

NMR: ¹H (300 MHz) (D₂O) δ 8.30 (s, 1H), 7.46 (m, 3H), 7.26 (m, 7H), 3.65 (d, 1H, J=12.9 Hz), 3.38 (m, 3H), 3.38 (m, 6H), 2.97 (m, 3H), 1.97 (m, 3H), 1.93 (q, 2H, J=7.5 Hz), 1.78 (m, 3H), 0.85 (t, 3H, J=7.5 Hz) pap.

MS: (ESI positive ion) m/z 447 (M+)

### Example 6

### Synthesis of N-Phosphonooxymethyl Pentazocine

Preparation of Mono-tert-butyl-N-Phosphonooxymethyl Pentazocine: To 50 mg (0.179 mmol) of pentazocine was added a solution of 65 mg (0.251 mmol) di-tert-butyl chloromethylphosphate plus 55 mg (0.354 mmol) of 1,2,2,6,6-pentamethylpiperidine in 5 mL of anhydrous acetonitrile (dried over 3 A molecular sieves), flushed with argon and sealed. The solution was stirred in a 45° C. oil bath for 2 days followed by 2^{nd} addition of 65 mg (0.251 mmol) of di-tert-butyl chloromethylphosphate plus 55 mg (0.35 mmol) of 1,2,2,6,6-pentamethylpiperidine and flask flushed with argon, sealed and stirred for 2 days in a 45° C. oil bath. It was then followed by a third addition of 65 mg (0.25 mmol) of di-tert-butyl chloromethylphosphate plus 55 mg (0.35 mmol) of 1,2,2,6,6-pentamethylpiperidine and the sealed flask under argon stirred in the oil bath at 45° C. for 3 more days. The reaction was evaporated to an oil and 20 mL of ethyl ether added with stirring. The white solid was filtered and vacuo dried (hygroscopic) to yield 100 mg (105%). The HPLC and MS (ESI+) showed a mixture of mono-t-butyl (major), di-t-butyl, t-butyl free compound, and pentazocine starting material.

Preparation of N-Phosphonooxymethyl Pentazocine: the crude mono-t-Butyl-pentazocine-methylphosphate in 10 mL of acetonitrile/1% TFA/H₂O (1/3) was stirred at room temperature for 6 days until MS showed that most of t-butyl had been removed. The solution was evaporated and the phosphate was purified on a Waters' Sunfire C₁₈ OBD prep column (10μ, 10×150 mm) eluting with 0.0025 ammonium formate (pH=6.5)/acetonitrile using a gradient of 80/20 to 60/40. With one injection, the largest peak was collected and evaporated to a glass at room temperature. The HPLC showed ˜12% impurity. The residue after triturating with acetonitrile gave 10 mg (14% yield) of desired product.

NMR: ¹H (300 MHz) (D₂O) δ 8.30 (s, 1H), 7.20 (d, 1H, J=8.4 Hz), 6.75 (s, 1H), 6.67 (d, 1H, J=8.4 Hz), 5.2 (d, 1H, J=8.4 Hz), 5.26 (do, 1H, J=10.0, 2.0H), 5.17 (t, 1H, J=7.0 Hz), 4.94 (t, 1H, J=6.9 Hz). 4.92 (dd, he, J=6.7, 1.0 Hz), 4.23 (m, 2H), 3.35 (m, 3H), 3.14 (dt, 1H, J=4.1, 17.8 Hz), 3.10 (s, 3H), 2.83 (dd, 1H, J=6.3, 20.3 Hz), 2.40 (dt, 1H, J=4.7, 14.7 Hz), 1.86 (dd, 1H, J=12.2, 2.7) pap

NMR: ¹³C (75 MHz) (D₂O) δ 155.1, 149.3, 141.2, 129.2, 124.5, 114.5, 112.5, 109.0, 75.5, 63.1, 50.4, 48.0, 36.0, 35.3, 35.1, 25.7, 24.3, 18.2, 13.3 pap

MS: (ESI positive ion) m/z 396 (M+)

### Example 7

### Synthesis of N-Phosphonooxymethyl Tramadol

Preparation of Mono-tert-butyl-N-Phosphonooxymethyl Tramadol: To 2 g (8.5 mmol; 1.0 eel) of treadle free base dissolved in 20 mL of acetonitrile, was added 1.5 g (5.7 mmol; 0.68 eel) di-tert-butyl chloromethylphosphate plus 1.05 g (6.8 mmol; 0.80 eel) of 1,2,2,6,6-pentamethylpiperidine. The solution was stirred for 5 days at 40° C., after that solvent was evaporated and resulting residue washed with diethyl ether and the crude product purified by preparative HPLC to yield 0.5 g of product (14% yield).

Preparation of N-Phosphonooxymethyl Tramadol: To 0.5 g (1.2 mmol; 1.0 eq) of Mono-tert-butyl-N-Phosphonooxymethyl tramadol in 10 mL of acetonitrile, was slowly added 3 mL TFA/Water (2:1) and stirred for 12 hours at room temperature. The solvent was then evaporated and resulting residue washed with diethyl ether and acetonitrile yielding 100 mg (25% yield) off-white solid.

NMR: ¹H (400 MHz) (DMSO) δ 7.25 (t, 1H), 7.14 (d, 1H), 7.10 (s, 1H), 6.90 (d, 1H), 3.85 (s, 3H), 3.00 (d, 2H), 2.60 (s, 2H), 2.25 (s, 1H), 2.21 (t, 2H), 1.90 (m, 2H) 1.50 (m, 4H) ppm

### Example 8

### Synthesis of N-Phosphonooxymethyl DiPOA

Preparation of Di-tert-butyl-N-Phosphonooxymethyl DiPOA: To 0.25 g (0.51 mmol; 1.0 eq) of DiPOA dissolved in 10 mL of acetonitrile, was added 0.133 g (0.51 mmol; 1.0 eq) di-tert-butyl chloromethylphosphate plus 0.06 g (0.41 mmol; 0.8 eq) of 1,2,2,6,6-pentamethylpiperidine. The solution was stirred for 5 days at 40° C., after that solvent was evaporated and resulting residue washed with diethyl ether and the crude product purified by preparative HPLC to yield 0.1 g (27% yield) of product as a white solid.

Preparation of N-Phosphonooxymethyl DiPOA: To 0.10 g (0.14 mmol; 1.0 eq) of Di-tert-butyl-N-Phosphonooxymethyl DiPOA in 10 mL of acetonitrile, was slowly added 3 mL TFA/Water (2:1) and stirred for 12 hours at room temperature. The solvent was then evaporated and resulting residue washed with diethyl ether and acetonitrile yielding 50 mg (62.5% yield) off-white solid.

NMR: ¹H (400 MHz) (DMSO) δ 7.45 (m, 1H), 7.29 (m, 1H), 7.23 (d, 1H), 6.69 (m, 1H), 6.80 (d, 1H), 5.60 (d, 2H), 4.90 (s, 2H), 4.20 (s, 2H), 3.95 (m, 1H), 3.25 (m, 4H), 2.99 (m, 4H), 2.60 (m, 2H), 1.99 (m, 2H) ppm

### Example 9

### Synthesis of N-(phosphonooxymethyl)Codeine

Preparation of Mono-tert-butyl-N-(phosphonooxymethyl)Codeine: To 57 mg (0.190 mmol) of codeine was added to a solution of 72 mg (0.278 mmol) di-tert-butyl chloromethylphosphate plus 55 mg (0.354 mmol) of 1,2,2,6,6-pentamethylpiperidine in 5 mL anhydrous acetonitrile (dried over 3 Å molecular sieves), flushed with argon, and sealed with a stopper. The solution was stirred in a 45° C. oil bath for 4 days. Mass spectral analysis of the reaction mixture showed starting material plus strong mono-t-butyl phosphate, weak di-t-butyl, and weak deblocked phosphoric acid compound. A second addition of 75 mg (0.290 mmol) di-tert-butyl chloromethylphosphate plus 55 mg (0.35 mmol) of 1,2,2,6,6-pentamethylpiperidine flushed with argon and the flask was sealed and stirred for 3 days in 43° C. in an oil bath. The mass spectrum was about the same. A third addition of 65 mg (0.25 mmol) di-tert-butyl chloromethylphosphate plus 55 mg (0.35 mmol) of 1,2,2,6,6-pentamethylpiperidine was added and the sealed flask under argon was stirred on the oil bath at 43° C. for four more days. The reaction was evaporated to an oil and 20 mL ethyl ether added with stirring. A gummy white, solid was separated from the ether mixture to yield 125 mg (160%). HPLC and MS (ESI+) showed a ˜1 to 1 mixture of mono-t-butyl and codeine plus some di-t-butyl and the free codeine-Me-phosphoric acid, and 1,2,2,6,6-pentamethylpiperidine.

Preparation of N-(Phosphonooxymethyl)Codeine: the crude mono-t-butyl-codeine-Me-phosphate in 10 mL acetonitrile/1% TFA/water (1/3) was stirred at room temperature for 4 days until mass spectrum showed that most of t-butyl had been removed. The solution was evaporated and the phosphate was purified on a Waters' Sunfire C18 OBD prep column (10 mu, 10×150 mm) eluting with 0.03 N ammonium formate (pH=6.2)/acetonitrile. A gradient of 99/1 to 60/40 was used. With one injection, the largest first peak of correct uv was collected and evaporated to a glass at room temperature to give 15 mg of solid (NMR shows strong pentamethylpiperidine peak).

NMR: ¹H (400 MHz) (D₂O) 6.71 (d, 1H, J=8.4 Hz), 6.58 (d, 1H, J=8.4 Hz), 5.53 (m, 1H), 5.22 (m, 1H), 5.10 (t, 1H J=13.0 Hz), 4.85-5.00 (m, 2H), 4.20 (m, 2H), 3.64 (s, 3H) OMe, 3.05-3.45 (m, 3H), 2.65-2.90 (m, 2H), 2.57 (s, 3H) NMe, 2.31-2.50 (m, 1H), 1.75-1.95 (m, 1H) ppm.

MS: (ESI positive ion) m/z 410 (M+).

### Example 10

### Synthesis of N-(phosphonooxymethyl)Hydrocodone

Extraction of Hydrocodone: A sample of 350 mg (0.71 mmol) of hydrocodone ditartrate salt was dissolved in 20 mL of 10% sodium bicarbonate and extracted with 3×30 mL of chloroform. The chloroform was dried over magnesium sulfate, filter, and evaporated to dryness to yield 196 mg (92%) of white solid. NMR

Preparation of Mono-tert-butyl-N-(phosphonooxymethyl) Hydrocodone: to 196 mg (0.654 mmol) of hydrocodone was added to a solution of 190 mg (0.734 mmol) di-tert-butyl chloromethylphosphate plus 120 mg (0.77 mmol) of 1,2,2,6,6-pentamethylpiperidine in 5 mL anhydrous acetonitrile (dried over 3 A molecular sieves), flushed with argon and sealed with a stopper. The solution was stirred on 45° C. oil bath for 4 days. Mass spectral analysis showed starting material plus strong mono-t-butyl phosphate, weak di-t-butyl and weak deblocked phosphoric acid compound. A second addition of 160 mg (0.618 mmol) di-tert-butyl chloromethylphosphate plus 100 mg (0.64 mmol) of 1,2,2,6,6-pentamethylpiperidine flushed with argon and the sealed flask was stirred for 3 days in 43° C. oil bath. The mass spectrum was about the same. A third addition of 65 mg (0.25 mmol) di-tert-butyl chloromethylphosphate plus 55 mg (0.35 mmoles) of 1,2,2,6,6-pentamethylpiperidinewas added and the sealed flask under argon stirred on the oil bath at 43° C. for three additional days. The reaction was evaporated to an oil and 20 mL ethyl ether added with stirring. A gummy white solid was separated from the ether mixture to yield 250 mg (85%). HPLC and MS (ESI+) showed a ˜1 to 1 mixture of mono-t-butyl and hydrocodone plus some di-t-butyl and the free hydrocodone-Me-phosphoric acid, and 1,2,2,6,6-pentamethylpiperidine.

Preparation of N-(phosphonooxymethyl) Hydrocodone: the crude mono-t-Bu-hydrocodone-Me-phosphate in 10 mL acetonitrile/1% TFA/water (1/3) was stirred at room temperature for 4 days until mass spectrum showed that most of t-butyl had been removed. The solution was evaporated and the phosphate was purified on a Waters' Sunfire C18 OBD prep column (10 mu, 10×150 mm) eluding with 0.03 N ammonium formate (pH=6.2)/acetonitrile. A gradient of 95/5 to 60/40 was used. With two injections, the largest first peak of correct UV was collected and evaporated to a glass at room temperature. 35 mg solid (NMR shows strong pentamethylpiperidine peak, which is very difficult to remove because of the polarity of the product).

NMR: ¹H (400 MHz) (D₂O) 6.84 (d, 1H, J=8.4 Hz), 6.76 (d, 1H, J=8.4 Hz), 5.02 (s, 1H), 4.73 (m, 2H), 4.03 (m, 1H), 3.73 (s, 3H) OMe, 3.20-3.40 (m, 2H), 3.10-3.21 (m, 2H), 2.70-3.00 (m, 3H), 2.61 (s, 3H) NMe, 2.52 (m, 1H), 1.86 (m, 2H) ppm.

MS: (ESI positive ion) m/z 410 (M+).

### Example 11

### Attempted Synthesis of N-Phosphonooxymethyl Oxycodone

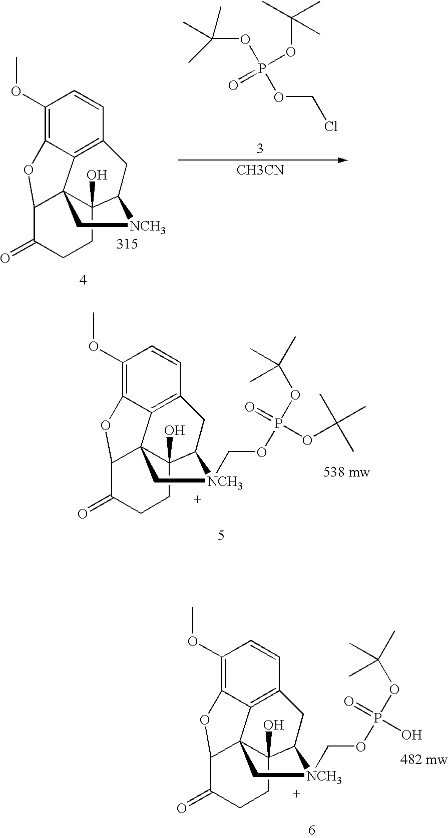

Di-tert-butyl chloromethyl Phosphate (3) and Oxycodone (4). 250 mg (0.712 mmol) of oxycodone hydrochloride was mixed with 50 mL ether and 25 mL sodium bicarbonate solution. The mixture was shaken and the ether layer separated. Three more times the 50 ml of ether was added until all the white solid had dissolved. The combined ether layers were dried over magnesium sulfate, filtered and evaporated to dryness (200 mg, 0.64 mmol). Residue was dissolved in 10 mL dry acetonitrile (dried over molecular sieves) and under argon added to 350 mg of the 85% chloromethylphosphate (˜1.15 mmol). The reaction mixture under argon in a glass bomb with stirring bar was heated in a 40° C. oil bath and stirred overnight. The reaction was evaporated to dryness and a NMR in CDCl₃ showed only starting materials.

Run 2. The above residue was dissolved in 10 mL of dry acetonitrile and under argon was added 178 mg (1.15 mmol) of 1,2,2,6,6-pentamethylpiperidine in 1 ml acetonitrile. The reaction in the glass bomb was stirred in an oil bath at 60° C. for three days. The reaction mixture was evaporated to dryness ethyl ether added (10 mL). The white solid was filtered and the filtrate evaporated to dryness. The white solid (185 mg) gave a MS of 316 (M+1) of oxycodone. The filtrate gave 316 peaks and a 532 peak but no 538 peak of product or any 482 of mono-t-butyl product. NMR of the filtrate did not show the 5.63 ppm doublet of chloromethylphosphate SM.

Run 3. The above solid (185 mg, 59 mmol) was dissolved in 10 mL of dry acetonitrile and under argon was added 178 mg (1.15 mmol) of 1,2,2,6,6-pentamethylpiperidine in 1 ml acetonitrile and 350 mg of the chloromethylphophate. The reaction in the glass bomb was stirred in an oil bath at 90-100° C. for three days. The reaction mixture was evaporated to dryness ethyl ether added (10 mL). The white solid was filtered and the filtrate evaporated to dryness. The white solid (155 mg) gave a MS of 316 (M+1) of oxycodone. The filtrate gave 316 peak and a 532 peak but no 538 peak or 482 of product. NMR of the filtrate did not show the 5.63 ppm doublet of chloromethylphosphate SM, but did show phosphate peaks. The residue was placed on prep thick TLC plate and developed with 20% MeOH/CH₂Cl₂ to give three bands. The bands were extracted with 20 MeOH/CHCl₃. None of the bands had any ³¹P in NMR.

Run 4. Using a new 210 mg (0.67 mmol) of oxycodone (free base) plus a new 350 mg of the chloromethylphosphate was added 176 mg of pentamethylpiperidine and I crystal of sodium iodide in 5 mL acetonitrile under argon. The mixture in glass bomb was heated on oil bath with stirring at 90-100° C. for 18 hrs. Solution was complete at start but a yellow precipitate form overnight. The mixture was cooled and filtered. The yellow solid (195 mg) gave MS of 316 of oxycodone and the filtrate gave MS 316 and 532 as before.

Run 5. The yellow solid was dissolved in chloroform and washed with sodium bicarbonate solution, dried over magnesium sulfate, filtered and evaporated to a solid (192 mg). The solid plus 350 mg of chloromethylphosphate and 178 mg pentamethylpiperidine was place in 40 ml methyl ethyl ketone and refluxed for 3 days. The solution was evaporated and MS showed 316 of oxycodone and the 532 peak. NMR showed some of the chloromethylphosphate doublet indicating not all of the phosphate had not decomposed.

Run 6. A 100 mg of recovered oxycodone plus 175 mg of the chloromethylphosphate was refluxed in 50 mL of methyl ethyl ketone for 3 days with the same results as 5th run.

Run 7. A 5 mg of oxycodone (free base) was refluxed in MEK with 3 drops methyl iodide for 3 days. MS showed a little 330 peak indicating some reaction but mostly SM. The reaction was continued with additional methyl iodide. After 2 days the reaction was evaporated and MS did not show any new 330 peak.

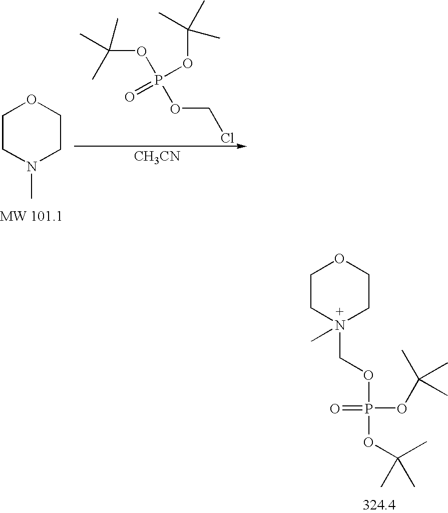

Run 8. A 25 mg ((0.27 mmol) of morpholine plus 127 mg (0.39 mmol) of di-tert-butyl chloromethyl Phosphate (3), in 5 mL of acetonitrile was heated in glass bomb with stirring in a 45° C. oil bath. After 18 hrs the reaction was evaporated to dryness to give oil. MS (ESI) of the product showed a strong peak of 324 indicating that the chlorophosphate would react with simple ring N-methyl cpd.

Run 9. A 5 mg (0.016 mmol) of oxycodone (4), 10 mg (0.038 mmol) di-tert-butyl chloromethyl Phosphate (3) in 2 mL of acetonitrile was stirred and heated in a Microwave for 10 min @ initial 140 then 100° C. (minimum conditions for temperature). Evaporation showed only 316 in MS of oxycodone (SM) and no 538 or 482 of possible products.

Run 10. To 164 mg (0.52 mmol) of oxycodone was added to a solution of 210 mg (0.81 mmol) di-tert-butyl chloromethylphosphate plus 155 mg (1.0 mmol) of 1,2,2,6,6-pentamethylpiperidine and 15 mg sodium iodide in 5 mL anhydrous acetonitrile (dried over 3 A molecular sieves), flushed with argon, and sealed with a stopper. The solution was stirred in a 45° C. oil bath for 3 days. The mass spectrum showed a strong m/e of 316 for starting material. A second addition of 100 mg (0.386 mmol) of di-tert-butyl chloromethylphosphate plus 80 mg (0.51 mmol) of 1,2,2,6,6-pentamethylpiperidine was made and the flask flushed with argon and the sealed. Stirring was continued for 5 days in a 43° C. oil bath. The mass spectra of the reaction mixture showed starting materials and other impurities but no peaks at m/e 426 (product), 482 (mono-t-butyl) or 538 (di-t-butyl). The reaction was evaporated to an oil to yield 300 mg. The oil was dissolved in 50 mL of 80% of 0.1% TFA/20% acetonitrile and stirred for 2 days and the mass spectrum showed m/e of 316 for starting material and other peaks but no 426 for product.

### Example 12

### Attempted Synthesis of N-Phosphonooxymethyl Oxymorphone

Oxymorphone: Twenty tablets of oxymorphone HCl salt was ground to a powder and slurried in 100 mL of 10% sodium carbonate solution and extracted with 100 mL of chloroform. The mixture formed an emulsion and was filtered through Celite and the layers separated. The filter cake was extracted with three times with 100 mL chloroform and the combined filtrate was dried over magnesium sulfate, filtered, and evaporated to dryness to yield 365 mg of a yellow-white solid.

Mono-tert-butyl-N-(phosphonooxymethyl)oxymorphone. A sample of 361 mg (1.20 mmol) of oxymorphone was added to a solution of 335 mg (0.129 mmol) of di-tert-butyl chloromethylphosphate plus 210 mg (0.135 mmol) of 1,2,2,6,6-pentamethylpiperidine in 5 mL anhydrous acetonitrile (dried over 3 A molecular sieves), flushed with argon and sealed with a stopper. The solution was stirred on 45° C. oil bath for 3 days. A mass spectrum showed a weak m/e 467 peak of the mono-t-butyl compound and a weak m/e of 412 for product and a strong m/e of 302 for starting material. A second addition of 210 mg (0.811 mmol) di-tert-butyl chloro-methylphosphate plus 130 mg (0.84 mmol) of 1,2,2,6,6-pentamethylpiperidine flushed with argon and the sealed flask was stirred for 5 days in a 43° C. oil bath. The mass spectrum of the reaction showed starting material, but loss of most of the 412 and 467 peaks. The reaction was evaporated to an oil with a yield of 400 mg. The oil was dissolved in 75 mL of 80% 0.1 5 TFA/acetonitrile and stirred for 2 days and mass spectrum showed a m/e of 312 for starting material and other peaks but no m/e of 412 for product.

### Example 13

### Attempted Synthesis of N-Phosphonooxymethyl Buprenorphine

Buprenorphine. A 110 mg (1.89 mmol) sample of buprenorphine HCl salt was dissolved in 20 mL of 10% sodium carbonate and extracted with 3×30 mL of chloroform. The chloroform was dried over magnesium sulfate, filtered, and evaporated to dryness to yield 85 mg (85%) of white solid.

Mono-tert-butyl-N-(phosphonooxymethyl)buprenorphine. To 85 mg (0.182 mmol) of buprenorphine was added to a solution of 80 mg (0.309 mmol) di-tert-butyl chloromethylphosphate plus 65 mg (0.418 mmol) of 1,2,2,6,6-pentamethylpiperidine in 5 mL anhydrous acetonitrile (dried over 3 A molecular sieves), flushed with argon, and sealed with a stopper. The solution was stirred in a 45° C. oil bath for 3 days. Mass spectral analysis of the reaction mixture showed a strong m/e of 316 for starting material and a very weak 578 peak of possible product. A second addition of 100 mg (0.386 mmol) di-tert-butyl chloromethylphosphate plus 70 mg (0.45 mmol) of 1,2,2,6,6-pentamethylpiperidine flushed with argon and the sealed flask was stirred for 5 days in a 43° C. oil bath. The mass spectrum of the reaction showed a weak starting material and other impurities peaks, but no peaks at 578 (product), 634 (mono-t-butyl) or 690 (di-t-butyl). The reaction was evaporated to an oil to yield 100 mg. The oil was dissolved in 30 mL of 80% of 0.1% TFA/20% acetonitrile and stirred for 2 days. The mass spectrum showed a weak m/e of 467 for starting materials and other peaks, but no 578 for product. The longer that the di-tert-butyl chloromethylphosphate reaction ran the starting material appeared to decompose.

### Example 14

### Attempted Synthesis of N-Phosphonooxymethyl Methadone

Mono-tert-butyl-N-(phosphonooxymethyl) Methadone. To 80 mg (0.258 mmol) of methadone was added a solution of 90 mg (0.349 mmol) di-tert-butyl chloromethylphosphate plus 65 mg (0.419 mmol) of 1,2,2,6,6-pentamethylpiperidine in 5 mL anhydrous acetonitrile (dried over 3 Å moleculars sieves), flushed with argon and sealed with a stopper. The solution was stirred in a 45° C. oil bath for 3 days and a 2nd addition of 90 mg (0.349 mmol) di-tert-butyl chloromethylphosphate plus 65 mg (0.42 mmol) of 1,2,2,6,6-pentamethylpiperidine flushed with argon and the sealed flask was stirred for 2 days in a 45° C. oil bath and a third addition of 90 mg (0.35 mmol)di-tert-butyl chloromethylphosphate plus 65 mg (0.42 mmol) of 1,2,2,6,6-pentamethylpiperidine was added and the sealed flask sealed under argon stirred in the oil bath at 45° C. for 4 more days. The reaction was evaporated to an oil and 20 mL of ethyl ether added with stirring. The white solid was filtered and vacuo dried (hygroscopic) to yield 75 mg (75%). The HPLC and MS (ESI+) showed a mixture of mono-t-butyl, di-t-butyl, t-butyl free compound, and methadone starting material.

Methadone-Me-phosphate. The crude mono-t-butyl-methadone-methylphosphate in 10 mL acetonitrile/1% TFA/H₂O (1/3) was stirred at room temperature for 6 days until MS showed that most of t-butyl had been removed, but also a larger amount of methadone starting material. The solution was evaporated and the phosphate was purified on a Waters' Sunfire C₁₈ OBD prep column (10μ, 10×150 mm) eluting with 0.0025 ammonium formate (pH=6.5)/acetonitrile using a gradient of 80/20 to 60/40. With one injection, the peaks were collected. The MS showed no phosphate compounds. The above reactions were tried twice more and all showed phosphate compounds in MS until preparative HPLC was tried. No product was isolated from the chromatographies.

### Example 15

### Attempted Synthesis of N-Phosphonooxymethyl Naltrexone

Run 1 To a stirred solution of 0.5 g naltrexone in acetonitrile was added 0.27 g (1.2 eq) pentamethyl piperidine (1.2 eq) followed by 0.45 g of di-tert-butyl chloromethyl phosphate (1.2 eq) and heated to 43° C. for two days. After two days additional 1.2 eq of pentamethyl piperidine and 1.2 eq of di-tert-butyl chloromethyl phosphate were added and continued the heating further for five days. There was no formation of the product. The reaction was monitored by TLC and HPLC. The NMR of crude compound obtained after work up did not comply with desired product structure.

Run 2 To a stirred solution of 0.5 g naltrexone in acetonitrile, was added 0.27 g (1.2 eq) pentamethyl piperidine (1.2 eq) followed by 0.45 g of di-tert-butyl chloromethyl phosphate (1.2 eq) and heated to 43° C. for three days. After three days additional 1.2 eq of pentamethyl piperidine and 1.2 eq of di-tert-butyl chloromethyl phosphate were added and continued the heating further for three days. After three days another 1.2 eq of pentamethyl piperidine and 1.2 eq of di-tert-butyl chloromethyl phosphate was added and the reaction continued under heating for further three days. There was no formation of the product. The reaction was monitored by TLC and HPLC. The NMR of crude compound obtained after work up did not comply with desired product structure.

Run 3 To a stirred solution of 0.5 g naltrexone in acetonitrile, was added 0.27 g (1.2 eq) pentamethyl piperidine (1.2 eq) followed by 0.45 g of di-tert-butyl chloromethyl phosphate (1.2 eq) and heated to 43° C. for seven days. After seven days, additional 1.2 eq of pentamethyl piperidine and 1.2 eq of di-tert-butyl chloromethyl phosphate were added and reaction was allowed to continue under heating for seven more days. After that another 1.2 eq of pentamethyl piperidine and 1.2 eq of di-tert-butyl chloromethyl phosphate was added and the reaction continued under heating for further seven days. There was no formation of the product. The reaction was monitored by TLC and HPLC. The NMR of crude compound obtained after work up did not comply with desired product structure.

Run 4 To a stirred solution of 0.2 g naltrexone in acetonitrile, was added 1.2 eq of pentamethyl piperidine followed by 1.2 eq of di-tert-butyl chloromethyl phosphate and heated to 43° C. for seven days. After seven days, additional 1.2 eq of pentamethyl piperidine and 1.2 eq of di-tert-butyl chloromethyl phosphate were added and reaction continued under heating for seven days. There was no formation of the product. The reaction was monitored by TLC and HPLC. The NMR of compound obtained after Prep-HPLC did not comply with desired product structure.

Run 5 To a stirred solution of 100 mg naltrexone in acetonitrile, was added pentamethylpiperidine (1.2 eq), and 40 mg bromochlormethane was stirred at 45° C. for 5 days. There was no formation of product as monitored by TLC and crude MS.

Run 6 To a stirred solution of 100 mg naltrexone in acetonitrile, was added pentamethylpiperidine (1.2 eq), and 62 mg chloroiodomethane was stirred at 45° C. for 3 days. There was no formation of product as monitored by TLC and crude MS.

Run 7 To a stirred solution of 100 mg naltrexone in acetonitrile, was added pentamethylpiperidine (1.2 eq) and 1-Bromo-1-chloro ethane (1.2 eq) was stirred at 45° C. for 5 days. There was no formation of product as monitored by TLC and crude MS.

Run 8 To a stirred solution of 50 mg naltrexone in acetonitrile, was added DIPEA (1.2 eq) and chloroiodomethane (1.2 eq). The reaction was heated to 45° C. for 3 days. There was no formation of product as determined by TLC and crude MS.

Run 9 To a stirred solution of 50 mg naltrexone in acetonitrile, was added KI (1 eq), DIPEA(2 eq) and chloroiodomethane (1.2 eq). The reaction was heated to 45° C. for 2 days. There was no formation of product as determined by TLC and crude MS.

Run 10 To a stirred solution of 50 mg naltrexone in acetonitrile, was added KI (1 eq), Bu4N Br (catalyst) and chloroiodomethane (1.2 eq). The reaction was heated to 45° C. for 2 days. There was no formation of product as determined by TLC and crude MS.

Run 11 To a stirred solution of 50 mg naltrexone in DMF, was added DIPEA (2 eq) and chloroiodomethane heated (1.2 eq). The reaction was heated to 45° C. for 2 days. There was no formation of product as determined by TLC and crude MS.

Run 12 To a stirred solution of 50 mg naltrexone in acetonitrile, was added DBU (1 eq) and chloroiodomethane (1.2 eq). The reaction was heated to 45° C. for 2 days. There was no formation of product as determined by TLC and crude MS.

All references disclosed herein are incorporated herein in their entireties.

1. A compound comprising:
(a) a parent drug moiety; and,
(b) a prodrug moiety of the formula: 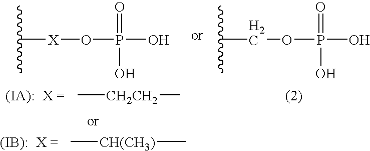
provided that when the prodrug moiety is of the formula (2), the parent drug moiety is not a moiety of an parent drug selected from the group consisting of levomethadyl, methadone, propoxyphene, buprenorphine, butorphanol, codeine, diphenoxylate, fentanyl, hydrocodone, hydromorphone, loperamide, meperidine, morphine, nalbuphine, nalmefene, naloxone, naltrexone, oxycodone, oxymorphone, pentazocine, sufentanil, alprazolam, clorazepate, clonazepam, estazolam, flurazepam, halazepam, lorazepam, midazolam, oxazepam, quazepam, temazepam and triazolam.

2. The compound of claim 1, wherein the prodrug moiety is of the formula (1A) or (1B) and the parent drug moiety is a moiety of an opiod, benzodiazepine, CNS drug, stimulant or anorexiant.

3. The compound of claim 2, wherein the compound is of the formula (I): 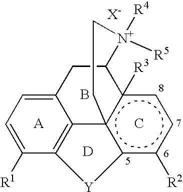
wherein:
R¹ is selected from the group consisting of hydrogen, C₁-C₁₀ alkanoate, hydroxyl and a substituted or unsubstituted C₁-C₁₀ alkyl and substituted or unsubstituted C₁-C₁₀ alkoxy;
R² is selected from the group consisting of hydrogen, ═O, hydroxyl, a substituted or unsubstituted C₁-C₁₀ alkyl, a substituted or unsubstituted C₂-C₁₀ alkenyl and a substituted or unsubstituted C₁-C₁₀ alkoxy;
R³ is selected from the group consisting of hydrogen, hydroxyl, C₁-C₁₀ alkanoate, a substituted or unsubstituted C₁-C₁₀ alkyl and a substituted or unsubstituted C₁-C₁₀ alkoxy;
R⁴ is selected from a group consisting of hydrogen, C₁-C₁₀ alkanoate, a substituted or unsubstituted C₁-C₁₀ alkyl, a substituted or unsubstituted C₂-C₁₀ alkenyl and a substituted or unsubstituted C₁-C₁₀ alkoxy;
R⁵ is the prodrug moiety (1A), (1B) or (2);
Y is null or is selected from O and S;
ring C has zero, one or two double bonds;
X⁻ is pharmaceutical acceptable anion;
or any stereoisomer, salt, hydrate or solvate thereof.

4. The compound of claim 2 or 3, wherein the prodrug moiety is of the formula (2).

5. The compound of claim 3, wherein R¹ is hydroxyl, R² and R³ are hydrogen, R⁴ is methyl, R⁵ is the prodrug moiety (1A) or (1B) and Y is null.

6. The compound of claim 3, wherein R¹ is hydroxyl, R² and R³ are hydrogen, R⁴ is methyl, R⁵ is the prodrug moiety (2) and Y is null.

7. The compound of claim 3, wherein R¹ is methoxy, R² is hydroxy, R³ is hydrogen, R⁴ is methyl, R⁵ is the prodrug moiety (1A) or (1B) and Y is oxygen and ring C has no double bond.

8. The compound of claim 3, wherein R¹ is methoxy, R² is hydroxy, R³ is hydrogen, R⁴ is methyl, R⁵ is the prodrug moiety (2) and Y is oxygen and ring C has no double bond.

9. The compound of claim 3, wherein R¹ is ethoxy, R² is hydroxy, R³ is hydrogen, R⁴ is methyl, R⁵ is the prodrug moiety (1A) or (1B) and Y is oxygen and ring C has one double bond between carbon 7 and 8.

10. The compound of claim 3, wherein R¹ is ethoxy, R² is hydroxy, R³ is hydrogen, R⁴ is methyl, R⁵ is the prodrug moiety (2) and Y is oxygen and ring C has one double bond between carbon 7 and 8.

11. The compound of claim 3, wherein R¹ is methoxy, R² is ═O, R³ is hydroxyl, R⁴ is methyl, R⁵ is the prodrug moiety (1A) or (1B) and Y is oxygen.

12. The compound of claim 3, wherein R¹ is methoxy, R² is ═O, R³ is hydroxyl, R⁴ is methyl, R⁵ is the prodrug moiety (1A) or (1B) and Y is oxygen.

13. The compound of claim 3, wherein R¹ is hydroxyl, R² is ═O, R³ is hydroxyl, R⁴ is methyl, R⁵ is the prodrug moiety (1A) or (1B) and Y is oxygen.

14. The compound of claim 3, wherein R¹ is hydroxyl, R² is ═O, R³ is hydroxyl, R⁴ is methyl, R⁵ is the prodrug moiety (2) and Y is oxygen.

15. The compound of claim 3, wherein R¹ is methoxy, R² is hydroxyl, R³ is hydrogen, R⁴ is methyl, R⁵ is the prodrug moiety (1A) or (1B), Y is oxygen and C₇ and C₈ are connected by a double bond.

16. The compound of claim 3, wherein R¹ is hydroxyl, R² is hydroxyl, R³ is hydrogen, R⁴ is methyl, R⁵ is the prodrug moiety (1A) or (1B), Y is oxygen and C₇ and C₈ are connected by a double bond.

17. The compound of claim 3, wherein R¹ is hydroxyl, R² is ═O, R³ is hydroxyl, R⁴ is cyclopropylmethyl and Y is O.

18. The compound of claim 3, wherein R¹ is hydroxyl, R² is ═O, R³ is hydroxyl, R⁴ is propen-3-yl and Y is O.

19. The compound of claim 3, wherein R¹ is hydroxyl, R² is ethenyl, R³ is hydroxyl, R⁴ is cyclopropylmethyl and Y is O.

20. The compound of claim 3, wherein R¹ is hydroxyl, R² is hydroxyl, R³ is hydrogen, R⁴ is propen-3-yl and Y is O.

21. The compound of claim 3, wherein R¹ is hydroxyl, R² is hydroxyl, R³ is hydroxyl, R⁴ is cyclobutylmethyl and Y is O.

22. The compound of claim 3, wherein R¹ is hydroxyl, R² is hydrogen, R³ is hydrogen, R⁴ is cyclopropylmethyl and Y is null.

23. The compound of claim 3, wherein R¹ is hydroxyl, R² is hydrogen, R³ is hydroxyl, R⁴ is cyclopropylmethyl and Y is null.

24. The compound of claim 3, wherein R¹ is hydroxyl, R² is hydrogen, R³ is hydrogen, R⁴ is propen-3-yl and Y is null.

25. The compound of claim 2, wherein the compound is of the formula (II): 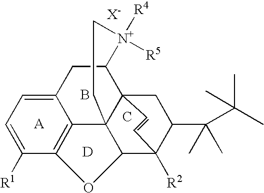
wherein:
R¹ is selected from the group consisting of hydrogen, hydroxyl, a substituted or unsubstituted C₁-C₁₀ alkyl and a substituted or unsubstituted C₁-C₁₀ alkoxy;
R² is selected from the group consisting of hydrogen, hydroxyl, a substituted or unsubstituted C₁-C₁₀ alkyl, a substituted or unsubstituted C₂-C₁₀ alkenyl and a substituted or unsubstituted C₁-C₁₀ alkoxy;
R⁴ is selected from a group consisting of hydrogen, a substituted or unsubstituted C₁-C₁₀ alkyl and a substituted or unsubstituted C₁-C₁₀ alkoxy;
R⁵ is the prodrug moiety (1A) or (1B) or (2);
Y is selected from a group consisting of null, O and S;
ring C has zero or one double bond;
X⁻ is pharmaceutical acceptable anion;
or any stereoisomer, salt, hydrate or solvate thereof.

26. The compound of claim 25, wherein R¹ is hydroxyl, R² is methoxy, R⁴ is cyclopropylmethyl, R⁵ is methoxyphosphonic acid and Y is oxygen.

27. The compound of claim 25, wherein R¹ is hydroxyl, R² is methoxy, R⁴ is cyclopropylmethyl, R⁵ is ethoxyphosphonic acid and Y is oxygen.

28. The compound of claim 2, wherein the compound is of the formula (III): 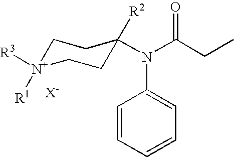
wherein:
R¹ is selected from the group consisting of hydroxyl, propylbenzene, ethylbenzene, 2-propylthiophene, methyl butyrate, 1-ethyl-4-ethyl-1H-tetrazol-5(4H)-one and 1-ethyl-4-propyl-1H-tetrazol-5(4H)-one, a substituted or unsubstituted C₁-C₁₀ alkyl and a substituted or unsubstituted C₁-C₁₀ alkoxy, alkylcarbonylalkoxy;
R² is selected from the group consisting of hydrogen, ═O, hydroxyl, a substituted or unsubstituted C₁-C₁₀ alkyl and C₁-C₁₀ alkoxy, C₁-C₁₀ alkanoate, C₂-C₁₀ alkoxyalkyl;
R³ is the prodrug moiety (1A), (1B) or (2);
X⁻ is a pharmaceutically acceptable anion;
or any stereoisomer, salt, hydrate or solvate thereof.

29. The compound of claim 28, wherein R¹ is propylbenzene, R² is hydrogen, and R³ is methoxyphosphonic acid.

30. The compound of claim 28, wherein R¹ is propylbenzene, R² is hydrogen, and R³ is ethoxyphosphonic acid.

31. The compound of claim 28, wherein R¹ is propylbenzene, R² is R² is methyl formoate, and R³ is methoxyphosphonic acid.

32. The compound of claim 28, wherein R¹ is propylbenzene, R² is R² is methyl formoate, and R³ is ethoxyphosphonic acid.

33. The compound of claim 28, wherein R¹ is 2-propylthiophene, R² is methoxy methyl, and R³ is methoxyphosphonic acid.

34. The compound of claim 28, wherein R¹ is 2-propylthiophene, R² is methoxy methyl, and R³ is ethoxyphosphonic acid.

35. The compound of claim 28, wherein R¹ is 1-ethyl-4-ethyl-1H-tetrazol-5(4H)-one, R² is methoxy methyl, and R³ is methoxyphosphonic acid.

36. The compound of claim 28, wherein R¹ is 1-ethyl-4-ethyl-1H-tetrazol-5(4H)-one, R² is methoxy methyl, and R³ is ethoxyphosphonic acid.

37. The compound of claim 28, wherein R¹ is methyl butyrate, R² is methyl formoate, and R³ is methoxyphosphonic acid.

38. The compound of claim 28, wherein R¹ is methyl butyrate, R² is methyl acetate, and R³ is ethoxyphosphonic acid.

39. A method of delaying the onset of parent drug activity in an individual in need of parent drug therapy, the method comprising administering to the individual an effective amount of a prodrug comprising a parent drug moiety and a prodrug moiety of the formula: 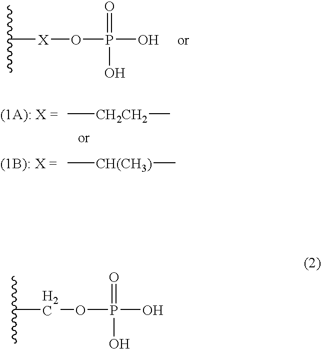
or any stereoisomer, salt, hydrate or solvate thereof, wherein the prodrug provides a slower onset of parent drug activity as compared to the parent drug.

40. A method of prolonging parent drug action in an individual in need of parent drug therapy, the method comprising administering to an individual an effective amount of a prodrug comprising a parent drug moiety and a prodrug moiety of the formula: 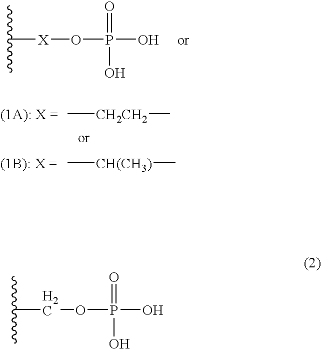
or any stereoisomer, salt, hydrate or solvate thereof, wherein the prodrug provides prolonged parent drug action as compared to the parent drug.

41. A method of decreasing the abuse potential of an APD in an individual in need of APD therapy, the method comprising administering to an individual an effective amount of a compound comprising an APD moiety and a prodrug moiety of the formula: 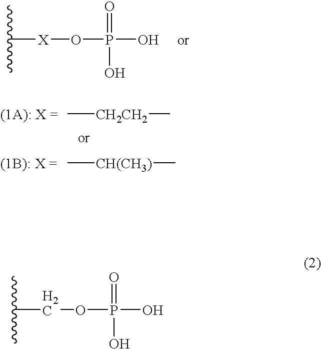
or any stereoisomer, salt, hydrate or solvate thereof, wherein the prodrug is less susceptible to abuse as compared to the parent APD.

42. The method of any one of claims 39-41, wherein the prodrug is selected from a prodrug of the formulae (I)-(IX) as detailed herein, or any stereoisomer, salt, hydrate or solvate thereof.

43. The compound of claim 2 or 3, wherein the prodrug moiety is of the formula (1A) or (1B).

44. A kit comprising: (a) an opioid prodrug comprising an opioid moiety and a prodrug moiety of the formula: 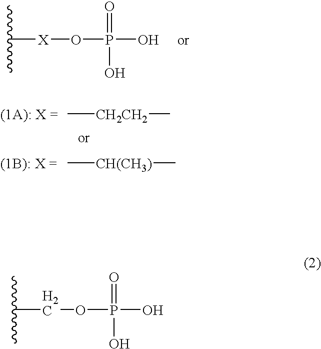
and (b) instructions for use of in the treatment, prevention, or delaying the onset and/or development of pain.

45. A pharmaceutical composition comprising (a) a prodrug comprising an parent drug moiety and a prodrug moiety of the formula: 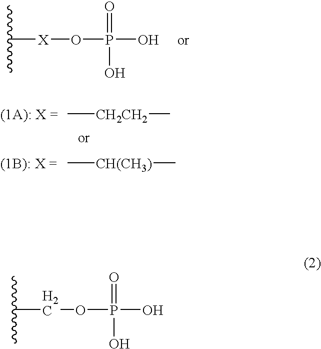
and (b) a pharmaceutically acceptable carrier.

46. The compound of claim 1, wherein the compound is of the formula (IV): 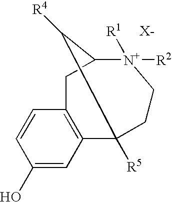
wherein R⁴ and R⁵ are independently alkyl; R² is the prodrug moiety (1A), (1B) or (2); R¹ is alkaryl or alkenyl and X⁻ is a pharmaceutically acceptable anion, and any stereoisomer, salt, hydrate or solvate thereof.

47. The compound of claim 44 wherein R⁴ and R⁵ are independently selected a substituted or unsubstituted C₁-C₅ alkyl; R² is the prodrug moiety (1A), (1B) or (2); R¹ is —(CH₂)ₙ-phenyl where n is selected from 1 to 5 or a C₂-C₁₀ alkenyl and X⁻ is a pharmaceutically acceptable anion, and any stereoisomer, salt, hydrate or solvate thereof.

48. The compound of claim 1, wherein the compound is of the formula (V): 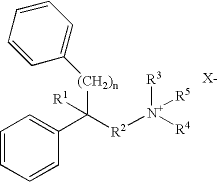
wherein R¹ is an alkanoate or acarbonylalkyl; R², R³ and R⁴ are independently a substituted or unsubstituted alkyl; R⁵ is the prodrug moiety (1A), (1B) or (2); n is an integer from 1 to 10 and X⁻ is a pharmaceutically acceptable anion, and any stereoisomer, salt, hydrate or solvate thereof.

49. The compound of claim 46 wherein R¹ is propanoate or propionyl; R², R³ and R⁴ are independently a substituted or unsubstituted C₁-C₅ alkyl; R⁵ is the prodrug moiety (1A), (1B) or (2); n is an integer from 1 to 5 and X⁻ is a pharmaceutically acceptable anion, and any stereoisomer, salt, hydrate or solvate thereof.

50. The compound of claim 1, wherein the compound is of the formula (VI): 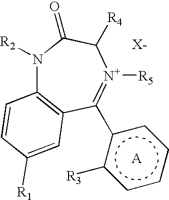
wherein:
R¹ is selected from the group consisting of bromine, chlorine, and nitro;
R² is selected from the group consisting of hydrogen and methyl;
R³ is selected from a group consisting of hydrogen and fluorine;
R⁴ is selected from a group consisting of hydrogen, and a carboxyl;
R⁵ is the prodrug moiety (1A), (1B) or (2);
or any stereoisomer, salt, hydrate or solvate thereof.

51. The compound of claim 50, wherein R¹ is chloride, R² is methyl, R³ is hydrogen, R⁴ is hydrogen and R⁵ the prodrug moiety (1A).

52. The compound of claim 50, wherein R¹ is chloride, R² is methyl, R³ is hydrogen, R⁴ is hydrogen and R⁵ the prodrug moiety (1B).

53. The compound of claim 50, wherein R¹ is chloride, R² is methyl, R³ is hydrogen, R⁴ is hydrogen and R⁵ the prodrug moiety (2).

54. The compound of claim 50, wherein R¹ is a nitro, R² is hydrogen, R³ is hydrogen, R⁴ is hydrogen and R⁵ the prodrug moiety (1A).

55. The compound of claim 50, wherein R¹ is a nitro, R² is hydrogen, R³ is hydrogen, R⁴ is hydrogen and R⁵ the prodrug moiety (1B).

56. The compound of claim 50, wherein R¹ is a nitro, R² is hydrogen, R³ is hydrogen, R⁴ is hydrogen and R⁵ the prodrug moiety (2).

57. The compound of claim 50, wherein R¹ is chloride, R² is methyl, R³ is hydrogen, R⁴ is hydrogen and R⁵ the prodrug moiety (1A).

58. The compound of claim 50, wherein R¹ is chloride, R² is methyl, R³ is hydrogen, R⁴ is hydrogen and R⁵ the prodrug moiety (1B).

59. The compound of claim 50, wherein R¹ is chloride, R² is methyl, R³ is hydrogen, R⁴ is hydrogen and R⁵ the prodrug moiety (2).

60. The compound of claim 50, wherein R¹ is a nitro, R² is methyl, R³ is fluoride, R⁴ is hydrogen and R⁵ the prodrug moiety (1A).

61. The compound of claim 50, wherein R¹ is a nitro, R² is methyl, R³ is fluoride, R⁴ is hydrogen and R⁵ the prodrug moiety (1B).

62. The compound of claim 50, wherein R¹ is a nitro, R² is methyl, R³ is fluoride, R⁴ is hydrogen and R⁵ the prodrug moiety (2).

63. The compound of claim 50, wherein R¹ is chloride, R² is hydrogen, R³ is hydrogen, R⁴ is acetyl and R⁵ the prodrug moiety (1A).

64. The compound of claim 50, wherein R¹ is chloride, R² is hydrogen, R³ is hydrogen, R⁴ is acetyl and R⁵ the prodrug moiety (1B).

65. The compound of claim 50, wherein R¹ is chloride, R² is hydrogen, R³ is hydrogen, R⁴ is acetyl and R⁵ the prodrug moiety (2).

66. The compound of claim 50, wherein R¹ is bromide, R² is hydrogen, R³ is hydrogen, R⁴ is hydrogen and R⁵ the prodrug moiety (1A).

67. The compound of claim 50, wherein R¹ is bromide, R² is hydrogen, R³ is hydrogen, R⁴ is hydrogen and R⁵ the prodrug moiety (1B).

68. The compound of claim 50, wherein R¹ is bromide, R² is hydrogen, R³ is hydrogen, R⁴ is hydrogen and R⁵ the prodrug moiety (2).

69. The compound of claim 1, wherein the compound is of the formula (VII): 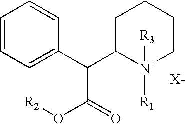
wherein:
R¹ is hydrogen,
R² is methyl;
R³ is the prodrug moiety (1A), (1B) or (2);
or any stereoisomer, salt, hydrate or solvate thereof.

70. The compound of claim 69, wherein R¹ is hydrogen, R² is methyl, and R³ is the prodrug moiety (1A).

71. The compound of claim 50, wherein R¹ is hydrogen, R² is methyl, and R³ is the prodrug moiety (1B).

72. The compound of claim 50, wherein R¹ is hydrogen, R² is methyl, and R³ is the prodrug moiety (2).

73. The compound of claim 1, wherein the compound is of the formula (VIII): 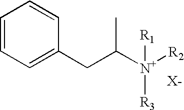
wherein:
R¹ is selected from the group consisting of hydrogen and methyl;
R² is hydrogen;
R³ is the prodrug moiety (1A), (1B) or (2);
or any stereoisomer, salt, hydrate or solvate thereof.

74. The compound of claim 73, wherein R¹ is hydrogen, R² is hydrogen, and R³ is the prodrug moiety (1A).

75. The compound of claim 73, wherein R¹ is hydrogen, R² is hydrogen, and R³ is the prodrug moiety (1B).

76. The compound of claim 73, wherein R¹ is hydrogen, R² is hydrogen, and R³ is the prodrug moiety (2).

77. The compound of claim 73, wherein R¹ is methyl, R² is hydrogen, and R³ is the prodrug moiety (1A).

78. The compound of claim 73, wherein R¹ is methyl, R² is hydrogen, and R³ is the prodrug moiety (1B).

79. The compound of claim 73, wherein R¹ is methyl, R² is hydrogen, and R³ is the prodrug moiety (2).

80. The compound of claim 1, wherein the compound is of the formula (IX): 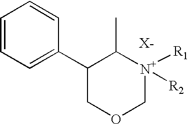
wherein:
R¹ is selected from the group consisting of hydrogen and methyl;
R² is the prodrug moiety (1A), (1B) or (2);
or any stereoisomer, salt, hydrate or solvate thereof.

81. The compound of claim 80, wherein R¹ is hydrogen and R² is the prodrug moiety (1A).

82. The compound of claim 73, wherein R¹ is hydrogen and R² is the prodrug moiety (1B).

83. The compound of claim 73, wherein R¹ is hydrogen and R² is the prodrug moiety (2).

84. The compound of claim 80, wherein R¹ is methyl and R² is the prodrug moiety (1A).

85. The compound of claim 73, wherein R¹ is methyl and R² is the prodrug moiety (1B).

86. The compound of claim 73, wherein R¹ is methyl and R² is the prodrug moiety (2).
